(19) United States
(12) Patent Application Publication
(10) Pub. No.: US 2013/0274281 A1
(43) Pub. Date: Oct. 17, 2013

(54) METHODS OF TREATING METASTATIC BREAST CANCER WITH 4-IODO-3-NITROBENZAMIDE AND IRINOTECAN

(71) Applicant: Charles Bradley, Half Moon Bay, CA (US)

(72) Inventor: Charles Bradley, Half Moon Bay, CA (US)

(73) Assignee: BiPar Sciences, Inc., Bridgewater, NJ (US)

(21) Appl. No.: 13/811,193

(22) PCT Filed: Jul. 19, 2011
(86) PCT No.: PCT/US11/44569

§ 371 Date: May 28, 2013

Related U.S. Application Data
(60) Provisional application No. 61/365,698, filed on Jul. 19, 2010.
(60) Provisional application No. 61/391,048, filed on Oct. 7, 2010.
(60) Provisional application No. 61/420,745, filed on Dec. 7, 2010.
(60) Provisional application No. 61/481,629, filed on May 2, 2011.
(60) Provisional application No. 61/486,660, filed on May 16, 2011.
(60) Provisional application No. 61/492,762, filed on Jun. 2, 2011.

(51) Int. Cl. A61K 31/4745; A61K 31/166

(52) U.S. Cl. CPC A61K 31/4745; A61K 31/166
USPC 514/283

(57) ABSTRACT

Provided herein are methods, compositions and kits for the treatment of locally advanced or metastatic breast cancer or breast cancer brain metastases. The method comprises administration of 4-iodo-3-nitrobenzamide, a metabolite or salt thereof in combination with irinotecan. The method of treating locally advanced or metastatic breast cancer comprises at least one 21 day treatment cycle.

### RELATED APPLICATIONS

This application claims the priority benefit of U.S. provisional applications U.S. Ser. No. 61/365,698, filed Jul. 19, 2010, U.S. Ser. No. 61/391,048, filed Oct. 7, 2010, U.S. Ser. No. 61/420,745, filed Dec. 7, 2010, U.S. Ser. No. 61/481,629, filed May 2, 2011, U.S. Ser. No. 61/486,660, filed May 16, 2011, and U.S. Ser. No. 61/492,762, filed Jun. 2, 2011, the contents of each of which are incorporated herein by reference in their entirety.

### TECHNICAL FIELD

The present invention relates to methods and compositions for the treatment of breast cancer comprising the administration of 4-iodo-3-nitrobenzamide or a metabolite thereof or a pharmaceutically acceptable salt thereof in combination with irinotecan.

### BACKGROUND

Cancer is a group of diseases characterized by aberrant control of cell growth. The annual incidence of cancer is estimated to be in excess of 1.3 million in the United States alone. While surgery, radiation, chemotherapy, and hormones are used to treat cancer, it remains the second leading cause of death in the U.S. It is estimated that over 560,000 Americans will die from cancer each year.

Cancer cells simultaneously activate several pathways that positively and negatively regulate cell growth and cell death. This trait suggests that the modulation of cell death and survival signals could provide new strategies for improving the efficacy of current chemotherapeutic treatments.

Breast cancer is generally treated with a combination of surgery to remove the cancerous lesion and adjuvant therapy—radiation, chemotherapy or both—to attack any cancer cells that may be left after the surgery. Breast cancer can be classified broadly by the presence or absence of hormone receptors (HRs). Hormone receptor positive (HR+) cancer is characterized by the expression of one or both female hormone receptors—estrogen receptor (ER) or progesterone receptor (PR).

Treatment with anthracycline is limited by lifetime dosing limits based on cardiotoxicity concerns. Treatment with gemcitabine and carboplatin is an established combination chemotherapy for metastatic breast cancer patients—whether taxane-naïve or taxane-pretreated. Platinum agents have demonstrated promising antitumor activity in basal-like locally advanced breast cancers. DNA damaging agents have promising antitumor efficacy against basal-like breast cancer because of defects in DNA repair pathways inherent in these breast cancers.

Despite the availability of antimetabolites such as gemcitabine and platinum complex agents such as carboplatin, there is no accepted standard of care for ER negative breast cancer. In particular, triple negative metastatic breast cancer (i.e., breast cancer that is ER negative, ands/or PR negative, and/or human epidermal growth factor receptor 2 (HER2) negative) is refractory to standard treatments and is entirely refractory to SERM chemotherapy. There is thus a need for an effective treatment for cancer in general, and especially for triple negative metastatic breast cancer.

Metastatic breast cancer is a complex multi-step process involving the expansion of cancerous cells from the breast to other areas of the body. It is a serious complication of breast cancer, as metastatic disease in breast cancer is often fatal, with treatments mainly limited to palliation.

Although there are limited therapeutic options for cancer treatment, variants of cancers, including metastatic breast cancer, triple negative breast cancer, are especially difficult because they can be refractory to standard chemotherapeutic or hormonal treatment. There is thus a need for an effective treatment for cancer in general, and cancer variants in particular.

### SUMMARY OF THE INVENTION

Provided herein are methods of treating breast cancer (e.g., locally advanced or metastatic breast cancer) in a patient, comprising administering to the patient an effective amount of (a) 4-iodo-3-nitrobenzamide or a metabolite thereof or a pharmaceutically acceptable salt thereof, and (b) irinotecan or a pharmaceutically acceptable salt thereof. In some embodiments, the method comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle. In some embodiments, the breast cancer is metastatic breast cancer. In some embodiments, the metastasis comprises brain metastases (e.g., brain metastasis measuring at least about 0.5 centimeter). In some embodiments, the breast cancer is locally advanced breast cancer. In some embodiments, the breast cancer is progressing locally advanced breast cancer.

In some embodiments, the breast cancer (e.g., locally advanced or metastatic breast cancer) is hormone receptor-negative (“HR-negative”) breast cancer. In some embodiments, the breast cancer (e.g., metastatic breast cancer) is negative for at least one of: estrogen receptor (“ER”), progesterone receptor (“PR”) or human epidermal growth factor receptor 2 (“HER2”). In some embodiments, the breast cancer (e.g., metastatic breast cancer) is negative for at least one of: ER, PR or HER2; and the breast cancer (e.g., metastatic breast cancer) is positive for at least one of ER, PR or HER2. In some embodiments, the breast cancer (e.g., metastatic breast cancer) is HR-negative breast cancer. In some embodiments, the breast cancer (e.g., metastatic breast cancer) is an ER-negative breast cancer. In some embodiments, the breast cancer (e.g., metastatic breast cancer) is ER-negative and HER2-positive. In some embodiments, the breast cancer (e.g., metastatic breast cancer) is ER-negative and PR-positive. In some embodiments, the breast cancer (e.g., metastatic breast cancer) is ER-negative and both HER2-positive and PR-positive. In some embodiments, the breast cancer (e.g., metastatic breast cancer) is a PR-negative breast cancer. In some embodiments, the breast cancer (e.g., metastatic breast cancer) is PR-negative and ER-positive. In some embodiments, the breast cancer (e.g., metastatic breast cancer) is PR-negative and HER2-positive. In some embodiments, the breast cancer (e.g., metastatic breast cancer) is PR-negative and both ER-positive and HER2-positive. In some embodiments, the breast cancer (e.g., metastatic breast cancer) is a HER2-negative breast cancer. In some embodiments, the breast cancer (e.g., metastatic breast cancer) is HER2-negative and ER-positive. In some embodiments, the breast cancer (e.g., metastatic breast cancer) is HER2-negative and PR-positive. In some embodiments, the breast cancer (e.g., metastatic breast cancer) is HER2-negative and both ER-positive and PR-positive. In some embodiments, the breast cancer (e.g., metastatic breast cancer) is ER-negative and PR-negative. In some embodiments, the breast cancer (e.g., metastatic breast cancer) is ER-negative, PR-negative and HER-2 positive. In some embodiments, the breast cancer (e.g., metastatic breast cancer) is ER-negative and HER2-negative. In some embodiments, the breast cancer (e.g., metastatic breast cancer) is ER-negative, HER2-negative and PR-positive. In some embodiments, the breast cancer (e.g., metastatic breast cancer) is PR-negative and HER2-negative. In some embodiments, the breast cancer (e.g., metastatic breast cancer) is PR-negative, HER2-negative and ER-positive. In some embodiments, the breast cancer (e.g., metastatic breast cancer) is ER-negative, PR-negative and HER2-negative. In some embodiments, the breast cancer (e.g., metastatic breast cancer) is ER-negative, PR-negative and HER2-nonoverexpressing.

In some embodiments, there is provided a method of treating locally advanced or metastatic breast cancer in a patient, comprising administering to the patient having locally advanced or metastatic breast cancer an effective amount of (a) 4-iodo-3-nitrobenzamide or a metabolite thereof or a pharmaceutically acceptable salt thereof, and (b) irinotecan or a pharmaceutically acceptable salt thereof, wherein the method comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle. In some embodiments, 4-iodo-3-nitrobenzamide or the pharmaceutically acceptable salt thereof is administered at about 8 mg/kg on days 1, 4, 8, and 11 of the 21-day cycle, and wherein irinotecan or a pharmaceutically acceptable salt thereof is administered at about 125 mg/m² on days 1 and 8 of the 21-day cycle.

In some embodiments, the patient has breast adenocarcinoma. In some embodiments, the breast cancer is locoregional breast cancer. In some embodiments, the breast cancer is progressing locoregional breast cancer. In some embodiments, the patient has distant metastasis. In some embodiments, the patient has systemic metastasis. In some embodiments, the breast cancer is refractory to standard therapy. In some embodiments, the patient has received prior chemotherapy treatment comprising at least one regimen selected from the group consisting of an anthracycline, an anthraquinone, and a taxane. In some embodiments, the patient is refractory to at least one regimen selected from the group consisting of an anthracycline, an anthraquinone, and a taxane. In some embodiments, the patient has lesion of at least 2.0 centimeter.

Also provided are methods of treating a patient with breast cancer brain metastasis comprising administering to the patient an effective amount of (a) 4-iodo-3-nitrobenzamide or a metabolite thereof or a pharmaceutically acceptable salt thereof and (b) irinotecan or a pharmaceutically acceptable salt thereof, wherein the breast cancer is ER-negative, PR-negative, and HER2-nonoverexpressing. In some embodiments, 4-iodo-3-nitrobenzamide or a pharmaceutically acceptable salt thereof (e.g., 4-iodo-3-nitrobenzamide) is administered to the patient. In some embodiments, irinotecan is administered to the patient. In some embodiments, there is provided a method of treating metastatic breast cancer brain metastasis in a patient comprising administering to the patient having the metastatic breast cancer brain metastasis an effective amount of (a) 4-iodo-3-nitrobenzamide or a metabolite thereof or a pharmaceutically acceptable salt thereof and (b) irinotecan, wherein the breast cancer is ER-negative, PR-negative, and HER2-nonoverexpressing. In some embodiments, the brain metastasis is at least about or larger than about 0.5 centimeter (e.g., brain metastasis measuring at least about or larger than about 0.5 centimeter in longest dimension). In some embodiments, the brain metastasis (e.g., brain metastasis measuring at least about or larger than about 0.5 centimeter) is new and/or progressive brain metastasis following radiation therapy (e.g., central nervous system (“CNS”) radiation therapy or intracranial radiation therapy). In some embodiments, the brain metastasis (e.g., brain metastasis measuring at least about or larger than about 0.5 centimeter) is new and/or progressive brain metastasis following radiation therapy (e.g., central nervous system (“CNS”) radiation therapy or intracranial radiation therapy) for breast cancer brain metastases. In some embodiments, the brain metastasis (e.g., brain metastasis measuring at least about or larger than about 0.5 centimeter) is new and/or progressive brain metastasis after prior radiation therapy (e.g., after prior central nervous system radiation therapy or after prior intracranial radiation therapy). In some embodiments, the brain metastasis (e.g., brain metastasis measuring at least about or larger than about 0.5 centimeter) is new and/or progressive brain metastasis after prior radiation therapy (e.g., after prior central nervous system radiation therapy or after prior intracranial radiation therapy) for breast cancer brain metastases. In some embodiments, the brain metastasis is new brain metastasis (e.g., new brain metastasis measuring at least about or larger than about 0.5 centimeter) after the radiation therapy. In some embodiments, the brain metastasis is progressive brain metastasis (e.g., progressive brain metastasis measuring at least about or larger than about 0.5 centimeter) after the radiation therapy. In some embodiments, the brain metastasis (e.g., brain metastasis measuring at least about or larger than about 0.5 centimeter) is asymptomatic and the patient is CNS-radiation therapy naïve patient. In some embodiments, the patient has no prior radiation therapy (e.g., prior intracranial radiation therapy). In some embodiments, the brain metastasis (e.g., brain metastasis measuring at least about or larger than about 0.5 centimeter) is new brain metastasis and the patient has no prior intracranial radiation therapy and/or the intracranial radiation therapy is not emergently indicated for the patient. In some embodiments, the brain metastasis (e.g., brain metastasis measuring at least about or larger than about 0.5 centimeter) is new brain metastasis (e.g., brain metastasis is found within 2 weeks of initiation of a therapy such as a protocol-based therapy) and the patient is intracranial radiation-naïve patient for whom intracranial radiation therapy is not emergently indicated. In some embodiments, the patient does not have leptomeningeal disease (e.g., the patient does not have diffuse leptomeningeal disease). In some embodiments, the effective amount is administered over a 21-day treatment cycle, wherein 4-iodo-3-nitrobenzamide or a metabolite thereof or a pharmaceutically acceptable salt thereof is administered to the patient at about 5.6 mg/kg on days 1, 4, 8, 11 of the treatment cycle, and wherein irinotecan is administered at about 125 mg/m² on days 1 and 8 of the cycle. In some embodiments, the effective amount is administered over a 21-day treatment cycle, wherein 4-iodo-3-nitrobenzamide or a metabolite thereof or a pharmaceutically acceptable salt thereof is administered to the patient at about 11.2 mg/kg on days 1 and 8 of the treatment cycle, and wherein irinotecan is administered at about 125 mg/m² on days 1 and 8 of the cycle.

In some embodiments, 4-iodo-3-nitrobenzamide or the metabolite thereof or the pharmaceutically acceptable salt thereof is administered intravenously. In some embodiments, irinotecan or the pharmaceutically acceptable salt thereof is administered intravenously.

In some embodiments, the method further comprises surgery, radiation therapy, chemotherapy, gene therapy, DNA therapy, viral therapy, RNA therapy, adjuvant therapy, neoadjuvant therapy, immunotherapy, nanotherapy or a combination thereof. In some embodiments, the radiation therapy comprises administering to the patient gamma irradiation.

In some embodiments, the effective amount produces at least one therapeutic effect selected from the group consisting of reduction in size of a breast tumor, reduction in metastasis, complete remission, partial remission, stable disease, increase in overall response rate, or a pathologic complete response. In some embodiments, the treatment produces complete response, partial response, or stable disease.

Also provided herein are uses of 4-iodo-3-nitrobenzamide, a metabolite thereof, or a pharmaceutically acceptable salt or solvate thereof, in combination with irinotecan, a pharmaceutically acceptable salt or solvate thereof for the manufacture of a medicament for the treatment or prevention of breast cancer (e.g., locally advanced breast cancer, metastatic breast cancer, breast cancer brain metastasis, or ER-negative, PR-negative, and HER2-nonoverexpressing breast cancer brain metastasis) described herein. Also provided herein are uses of 4-iodo-3-nitrobenzamide, a metabolite thereof, or a pharmaceutically acceptable salt or solvate thereof for the manufacture of a medicament for the treatment or prevention of breast cancer (e.g., locally advanced breast cancer, metastatic breast cancer, breast cancer brain metastasis, or ER-negative, PR-negative, and HER2-nonoverexpressing breast cancer brain metastasis) in combination with irinotecan, a pharmaceutically acceptable salt or solvate thereof described herein. In certain embodiments, the medicament is provided for the treatment of breast cancer (e.g., locally advanced breast cancer, metastatic breast cancer, breast cancer brain metastasis, or ER-negative, PR-negative, and HER2-nonoverexpressing breast cancer brain metastasis). Also provided herein are uses of -iodo-3-nitrobenzamide, a metabolite thereof, or a pharmaceutically acceptable salt or solvate thereof, in combination with irinotecan, a pharmaceutically acceptable salt or solvate thereof, for treatment of breast cancer (e.g., locally advanced breast cancer, metastatic breast cancer, breast cancer brain metastasis, or ER-negative, PR-negative, and HER2-nonoverexpressing breast cancer brain metastasis) in a patient. Also provided herein are synergistic compositions used for treating breast cancer (e.g., locally advanced breast cancer, metastatic breast cancer, breast cancer brain metastasis, or ER-negative, PR-negative, and HER2-nonoverexpressing breast cancer brain metastasis) in a patient comprising a) 4-iodo-3-nitrobenzamide, or a metabolite thereof, or a pharmaceutically acceptable salt or solvate thereof, and b) irinotecan, or pharmaceutically acceptable salt or solvate thereof, to said patient. The uses described herein may be in accordance with any of the methods described herein.

Also provided herein are kits for the treatment or prevention in a patient with breast cancer (e.g., locally advanced breast cancer, metastatic breast cancer, breast cancer brain metastasis, or ER-negative, PR-negative, and HER2-nonoverexpressing breast cancer brain metastasis), comprising 4-iodo-3-nitrobenzamide, a metabolite thereof, or a pharmaceutically acceptable salt or solvate thereof, in combination with irinotecan, or a pharmaceutically acceptable salt or solvate thereof. In some embodiments, the kit comprises instructions for using 4-iodo-3-nitrobenzamide, a metabolite thereof, or a pharmaceutically acceptable salt or solvate thereof in combination with irinotecan, or a pharmaceutically acceptable salt or solvate thereof for the treatment or prevention in a patient with breast cancer (e.g., locally advanced breast cancer, metastatic breast cancer, breast cancer brain metastasis, or ER-negative, PR-negative, and HER2-nonoverexpressing breast cancer brain metastasis) according to any of the methods described herein. Also provided herein are kits comprising 4-iodo-3-nitrobenzamide, a metabolite thereof, or a pharmaceutically acceptable salt or solvate thereof, and a label or packaging insert containing information and/or instructions related to use of 4-iodo-3-nitrobenzamide, a metabolite thereof, or a pharmaceutically acceptable salt or solvate thereof in combination with irinotecan, or a pharmaceutically acceptable salt or solvate thereof for the treatment or prevention in a patient with breast cancer (e.g., locally advanced breast cancer, metastatic breast cancer, breast cancer brain metastasis, or ER-negative, PR-negative, and HER2-nonoverexpressing breast cancer brain metastasis) according to any of the methods described herein. A kit described herein may comprise packaging. The dosage or dosing regimen for 4-iodo-3-nitrobenzamide, a metabolite thereof, or a pharmaceutically acceptable salt or solvate thereof can be any dosage or dosing regimen described herein. The dosage or dosing regimen for irinotecan, a pharmaceutically acceptable salt or solvate thereof can be any dosage or dosing regimen described herein.

For example, in some embodiments, there is provided a kit for treating a patient with breast cancer brain metastasis comprising (a) 4-iodo-3-nitrobenzamide or a metabolite thereof or a pharmaceutically acceptable salt thereof, and (b) irinotecan or a pharmaceutically acceptable salt thereof, wherein the breast cancer is ER-negative, PR-negative, and HER2-nonoverexpressing. In some embodiments, the kit further comprises instructions for using effective amount of (a) 4-iodo-3-nitrobenzamide or a metabolite thereof or a pharmaceutically acceptable salt thereof, and (b) irinotecan or a pharmaceutically acceptable salt thereof to treat the patient with breast cancer brain metastasis. In some embodiments, there is provided a kit comprising (a) 4-iodo-3-nitrobenzamide or a metabolite thereof or a pharmaceutically acceptable salt thereof, and (b) instructions for using an effective amount of 4-iodo-3-nitrobenzamide or a metabolite thereof or a pharmaceutically acceptable salt thereof, in combination with irinotecan or a pharmaceutically acceptable salt thereof to treat a patient with breast cancer brain metastasis, wherein the breast cancer is ER-negative, PR-negative, and HER2-nonoverexpressing. In some embodiments, there is provided a kit comprising (a) 4-iodo-3-nitrobenzamide or a metabolite thereof or a pharmaceutically acceptable salt thereof, (b) irinotecan or a pharmaceutically acceptable salt thereof, and (c) instructions for using 4-iodo-3-nitrobenzamide or a metabolite thereof or a pharmaceutically acceptable salt thereof, and irinotecan or a pharmaceutically acceptable salt thereof to treat locally advanced or metastatic breast cancer in a patient, wherein the treatment comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle. In some embodiments, there is provided a kit comprising (a) 4-iodo-3-nitrobenzamide or a metabolite thereof or a pharmaceutically acceptable salt thereof, and (b) instructions for using 4-iodo-3-nitrobenzamide or a metabolite thereof or a pharmaceutically acceptable salt thereof, in combination with irinotecan or a pharmaceutically acceptable salt thereof to treat locally advanced or metastatic breast cancer in a patient, wherein the treatment comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle.

Also provided are articles of manufacture comprising the compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed.

It is to be understood that one, some, or all of the properties of the various embodiments described herein may be combined to form other embodiments of the present invention. These and other aspects of the invention will become apparent to one of skill in the art.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the treatment scheme (3+3 dose escalation) for treating patients with metastatic breast cancer with 4-iodo-3-nitrobenzamide and irinotecan.

FIG. 2 shows best % change in tumor size from baseline in triple negative breast cancer (“TNBC”) and non-TNBC patients treated with 4-iodo-3-nitrobenzamide and irinotecan. The bars represent best single measurement of target lesion from each patient.

FIG. 3 shows immunohistochemistry (“IHC”) staining for BRCA1 on tumor samples measured with AQUA™ technology.

### DETAILED DESCRIPTION

### Definitions

As used herein, “treatment” or “treating” or its grammatical equivalents as used herein includes achieving beneficial or desired results including, e.g., a therapeutic benefit, a prophylactic benefit, and/or clinical results. For purposes of this invention, beneficial or desired results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, eradication of the underlying disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

For example, in an individual with breast cancer (e.g., metastatic breast cancer), a benefit (e.g., a therapeutic benefit) includes eradication or amelioration of the underlying breast cancer (e.g., metastatic breast cancer), e.g., slowing of progression of the breast cancer (e.g., metastatic breast cancer). Also, a therapeutic benefit is achieved with the eradication or amelioration of one or more of the physiological symptoms associated with the underlying disorder (e.g., breast cancer) such that an improvement is observed in the individual, notwithstanding the fact that the individual may still be afflicted with the underlying disorder (e.g., breast cancer). For a benefit such as prophylactic benefit, a method of the invention may be performed on, or a composition of the invention administered to an individual at risk of developing breast cancer, or to an individual reporting one or more of the physiological symptoms of breast cancer, even though a diagnosis of breast cancer may not have been made. In some embodiments, the individual being treated has been diagnosed with a breast cancer described herein.

The term “individual” or “patient” refers to a mammal and includes, but is not limited to, human, bovine, horse, feline, canine, rodent, or primate. Preferably, the individual is a human. An individual may be a patient.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer. An individual having one or more of these risk factors has a higher probability of developing cancer than an individual without these risk factor(s).

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

As used herein “surgery” refers to any therapeutic or diagnostic procedure that involves methodical action of the hand or of the hand with an instrument, on the body of a human or other mammal, to produce a curative, remedial, or diagnostic effect.

“Radiation therapy” refers to exposing an individual to high-energy radiation, including without limitation x-rays, gamma rays, and neutrons. This type of therapy includes without limitation external-beam therapy, internal radiation therapy, implant radiation, brachytherapy, systemic radiation therapy, and radiotherapy.

“Chemotherapy” refers to the administration of one or more anti-cancer drugs such as, antineoplastic chemotherapeutic agents, chemopreventative agents, and/or other agents to an individual with breast cancer (e.g., metastatic breast cancer) by various methods, including intravenous, oral, intramuscular, intraperitoneal, intravesical, subcutaneous, transdermal, buccal, or inhalation or in the form of a suppository. Unless clearly dictated otherwise by context, “chemotherapy” as used herein is not intended to refer to the administration of 4-iodo-3-nitrobenzamide and irinotecan. Chemotherapy may be given prior to surgery to shrink a large tumor prior to a surgical procedure to remove it, prior to radiation therapy, or after surgery and/or radiation therapy to prevent the growth of any remaining breast cancer cells in the body. Chemotherapy may also occur during the course of radiation therapy.

The terms “effective amount” or “pharmaceutically effective amount” refer to a sufficient amount of an agent to provide the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. For example, an “effective amount” for therapeutic uses is the amount of a) 4-iodo-3-nitrobenzamide or a metabolite thereof or a pharmaceutically acceptable salt or solvate thereof or b) irinotecan, or a pharmaceutically acceptable salt or solvate thereof, provided herein, or a composition comprising a) 4-iodo-3-nitrobenzamide or a metabolite thereof or a pharmaceutically acceptable salt thereof and b) irinotecan required to provide a clinically significant decrease in the breast tumor or slowing of progression of the breast cancer (e.g., metastatic breast cancer).

“Metabolite” refers to a compound produced through any in vitro or in vivo metabolic process which results in a product that is different in structure than that of the starting compound. In other words, the term “metabolite” includes the metabolite compounds of 4-iodo-3-nitrobenzamide, for example, 4-iodo-3-aminobenzoic acid (“IABA”) and 4-iodo-3-aminobenzamide (“IABM”). A metabolite can include a varying number or types of substituents that are present at any position relative to a precursor compound. In addition, the terms “metabolite” and “metabolite compound” are used interchangeably herein.

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

An “adverse event” or “AE,” unless specified otherwise, refers to any untoward medical occurrence in a patient receiving a marketed pharmaceutical product or in a patient who is participating on a clinical trial who is receiving an investigational or non-investigational pharmaceutical agent. The AE does not necessarily have a causal relationship with the patient's treatment. Therefore, an AE can be any unfavorable and unintended sign, symptom, or disease temporally associated with the use of a medicinal product, whether or not considered to be related to the medicinal product. An AE includes, but is not limited to: an exacerbation of a pre-existing illness; an increase in frequency or intensity of a pre-existing episodic event or condition; a condition detected or diagnosed after study drug administration even though it may have been present prior to the start of the study; and continuously persistent disease or symptoms that were present at baseline and worsen following the start of the study. An AE generally does not include: medical or surgical procedures (e.g., surgery, endoscopy, tooth extraction, or transfusion); however, the condition that leads to the procedure is an adverse event; pre-existing diseases, conditions, or laboratory abnormalities present or detected at the start of the study that do not worsen; hospitalizations or procedures that are done for elective purposes not related to an untoward medical occurrence (e.g., hospitalizations for cosmetic or elective surgery or social/convenience admissions); the disease being studied or signs/symptoms associated with the disease unless more severe than expected for the patient's condition; and overdose of study drug without any clinical signs or symptoms.

A “serious adverse event” or (SAE), unless specified otherwise, refers to any untoward medical occurrence at any dose including, but not limited to, that: a) is fatal; b) is life-threatening (defined as an immediate risk of death from the event as it occurred); c) results in persistent or significant disability or incapacity; d) requires in-patient hospitalization or prolongs an existing hospitalization (exception: Hospitalization for elective treatment of a pre-existing condition that did not worsen during the study is not considered an adverse event; complications that occur during hospitalization are AEs and if a complication prolongs hospitalization, then the event is serious); e) is a congenital anomaly/birth defect in the offspring of an individual who received medication; or f) conditions not included in the above definitions that may jeopardize the individual or may require intervention to prevent one of the outcomes listed above unless clearly related to the individual's underlying disease. “Lack of efficacy” (progressive disease) is not considered an AE or SAE. The signs and symptoms or clinical sequelae resulting from lack of efficacy should be reported if they fulfill the AE or SAE definitions.

The following definitions may be used to evaluate response based on target lesions: unless specified otherwise, “complete response” or “CR” (also known as “complete remission”) refers to disappearance of all target lesions; “partial response” or “PR” (also known as “partial remission”) refers to at least a 30% decrease in the sum of the longest diameters (SLD) of target lesions, taking as reference the baseline SLD; “stable disease” or “SD” refers to neither sufficient shrinkage of target lesions to qualify for PR, nor sufficient increase to qualify for PD, taking as reference the nadir SLD since the treatment started; and “progressive disease” or “PD” refers to at least a 20% increase in the SLD of target lesions, taking as reference the nadir SLD recorded since the treatment started, or, the presence of one or more new lesions.

The following definitions of response assessments may be used to evaluate a non-target lesion: unless specified otherwise, “complete response” or “CR” refers to disappearance of all non-target lesions; “stable disease” or “SD” refers to the persistence of one or more non-target lesions not qualifying for CR or PD; and “progressive disease” or “PD” refers to the “unequivocal progression” of existing non-target lesion(s) or appearance of one or more new lesion(s) is considered progressive disease (if PD for the subject is to be assessed for a time point based solely on the progression of non-target lesion(s), then additional criteria are required to be fulfilled).

“Progression free survival” (PFS) may indicate the length of time during and after treatment that the cancer does not grow. Progression-free survival may include the amount of time patients have experienced a complete response or a partial response, as well as the amount of time patients have experienced stable disease.

As used herein, “sample” refers to a composition which contains a molecule which is to be characterized and/or identified, for example, based on physical, biochemical, chemical, physiological, and/or genetic characteristics.

“Cells,” as used herein, is understood to refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein.

“HER2-negative” used herein means “HER2 non-overexpressing” as understood by one skilled in the art.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise.

It is understood that aspects and variations of the invention described herein include “consisting” and/or “consisting essentially of” aspects and variations.

As is apparent to one skilled in the art, an individual assessed, selected for, and/or receiving treatment is an individual in need of such activities.

### 4-iodo-3-nitrobenzamide or a metabolite thereof

4-iodo-3-nitrobenzamide, also known as iniparib or “BA,” has the formula:

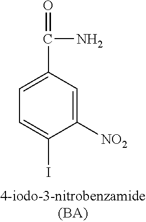 [SMILES: NC(=O)c1ccc(I)c([N+](=O)[O-])c1]

Methods of making 4-iodo-3-nitrobenzamide are known to the field, such as the methods disclosed in U.S. Pat. No. 5,464,871, which is hereby incorporated by reference in its entirety, particularly with respect to the synthetic methods disclosed therein.

Provided herein are precursor compounds of Formula (Ia)

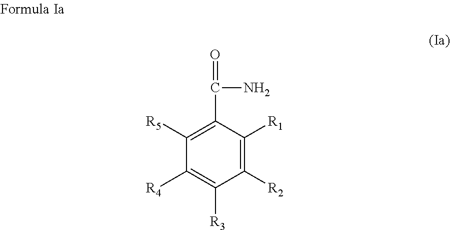

wherein R₁, R₂, R₃, R₄, and R₅ are, independently selected from the group consisting of hydrogen, hydroxy, amino, nitro, nitroso, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents is always nitro, and at least one substituent positioned adjacent to a nitro is always iodo, and pharmaceutically acceptable salts, solvates, isomers, tautomers, metabolites, analogs, or pro-drugs thereof. R₁, R₂, R₃, R₄, and R₅ can also be a halide such as chloro, fluoro, or bromo substituents. In some embodiments, at least one of the R₁, R₂, R₃, R₄, and R₅ substituents is always nitro or nitroso and at least one substituent positioned adjacent to the nitro or nitroso is always iodo. In some embodiments, the compound of formula Ia is a compound of formula IA or a metabolite or pharmaceutically acceptable salt, solvate, isomer, or tautomer thereof. In some embodiments, at least one of the R₁, R₂, R₃, R₄, and R₅ substituents is always nitro or nitroso and at least one substituent positioned adjacent to the nitro or nitroso is always iodo. In some embodiments, the compound of formula Ia is a compound of formula IA or pharmaceutically acceptable salt, solvate, isomer, or tautomer thereof.

Also provided herein are metabolites with the Formula (IIa):

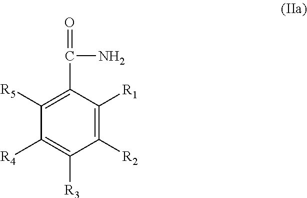

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent to a R₁, R₂, R₃, R₄ or R₅ group that is a nitroso, hydroxyamino, hydroxy or amino group. In some embodiments, the compounds of (2) are such that the iodo group is always adjacent to a R₁, R₂, R₃, R₄ or R₅ group that is a nitroso, hydroxyamino, or amino group.

Any of the compounds with structure formula Ia or IIa may be used for a treatment described herein. In some embodiments, the compound with structure formula Ia or IIa is 4-iodo-3-nitrobenzamide or a metabolite thereof or a pharmaceutically acceptable salt thereof.

Provided herein are metabolite compounds, each represented by a chemical formula:

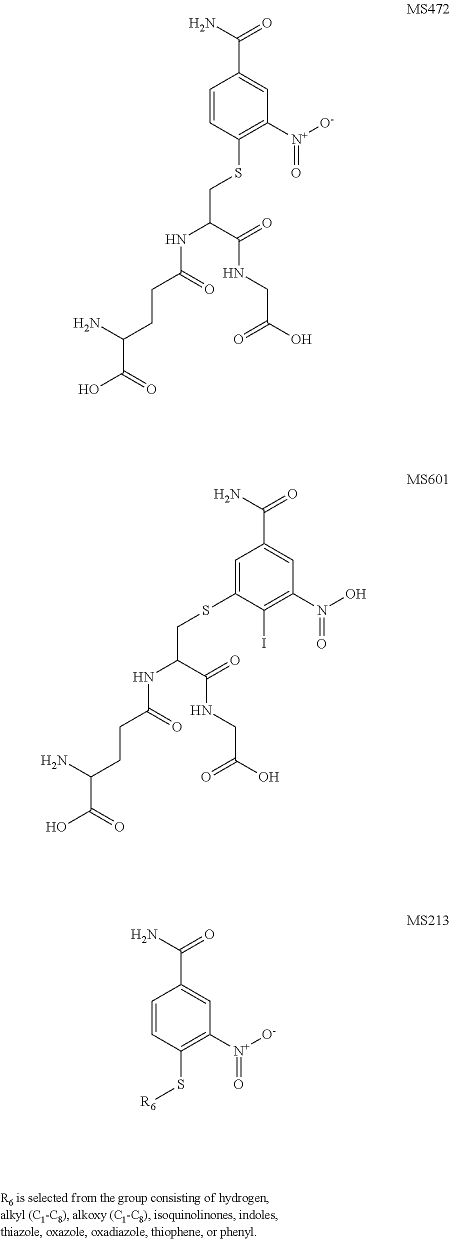

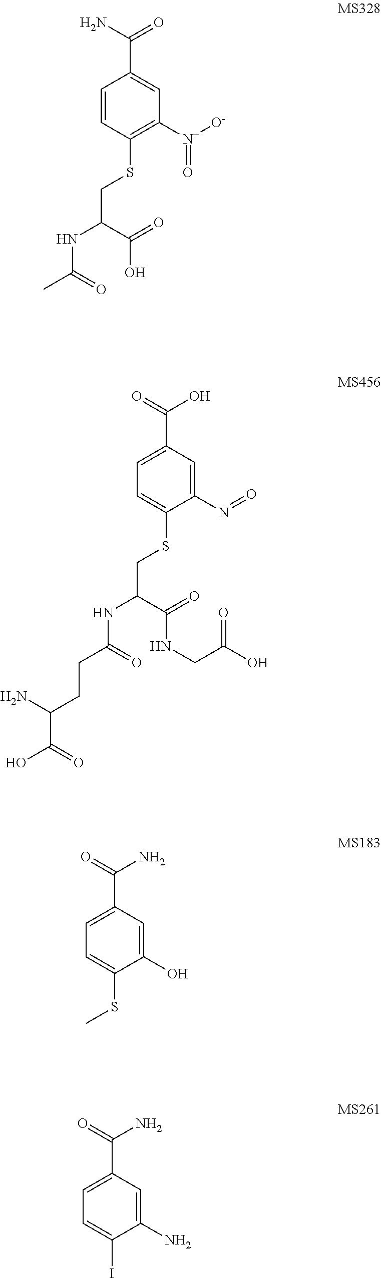 [SMILES: CC(=O)NC(CSc1ccc(C(N)=O)cc1[N+](=O)[O-])C(=O)O ; NC(CCC(=O)NC(CSc1ccc(C(=O)O)cc1N=O)C(=O)NCC(=O)O)C(=O)O ; CSc1ccc(C(N)=O)cc1O ; NC(=O)c1ccc(I)c(N)c1]

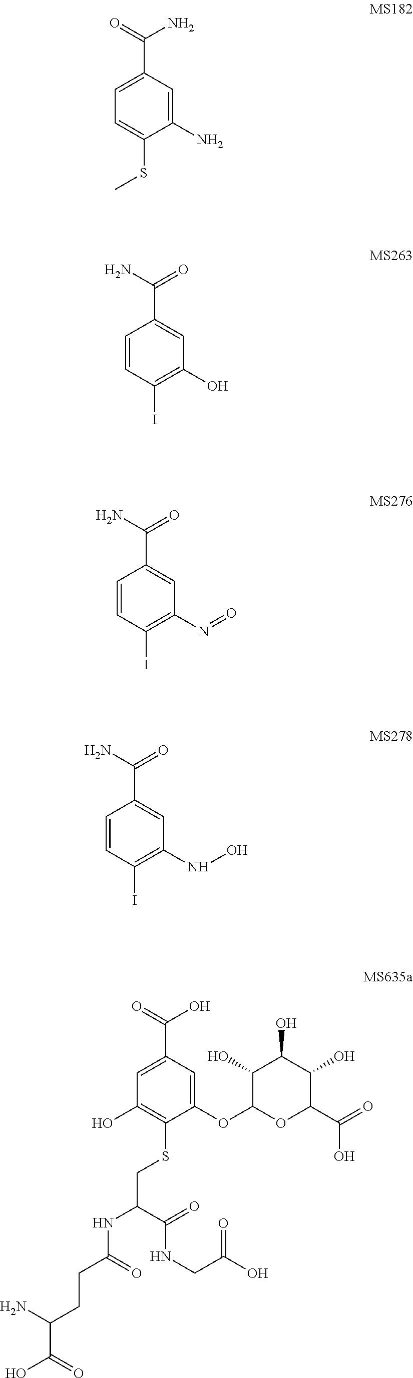 [SMILES: CSc1ccc(C(N)=O)cc1N ; NC(=O)c1ccc(I)c(O)c1 ; NC(=O)c1ccc(I)c(N=O)c1 ; NC(=O)c1ccc(I)c(NO)c1 ; NC(CCC(=O)NC(CSc1c(O)cc(C(=O)O)cc1OC1OC(C(=O)O)[C@@H](O)[C@H](O)[C@H]1O)C(=O)NCC(=O)O)C(=O)O]

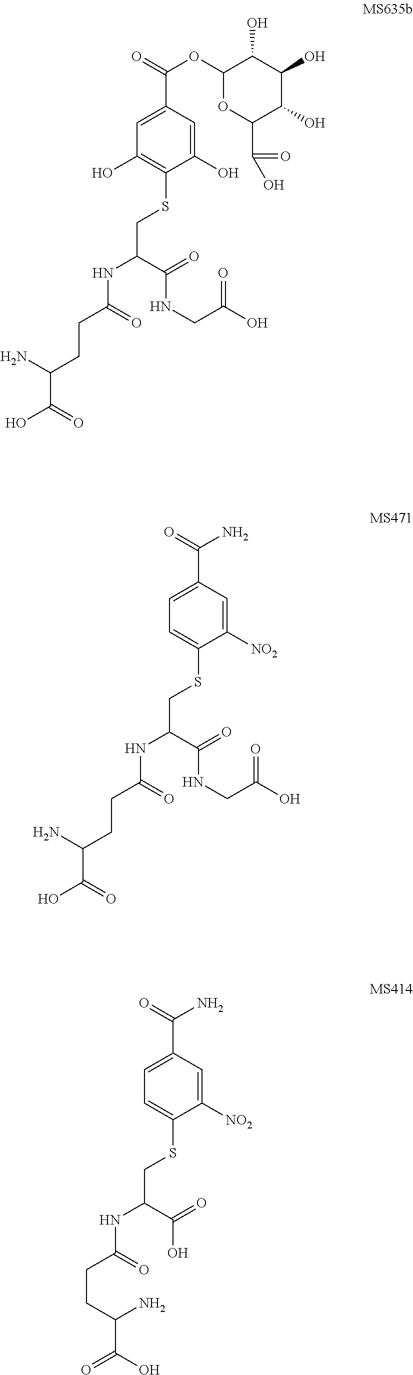 [SMILES: NC(CCC(=O)NC(CSc1c(O)cc(C(=O)OC2OC(C(=O)O)[C@@H](O)[C@H](O)[C@H]2O)cc1O)C(=O)NCC(=O)O)C(=O)O ; NC(=O)c1ccc(SCC(NC(=O)CCC(N)C(=O)O)C(=O)NCC(=O)O)c([N+](=O)[O-])c1 ; NC(=O)c1ccc(SCC(NC(=O)CCC(N)C(=O)O)C(=O)O)c([N+](=O)[O-])c1]

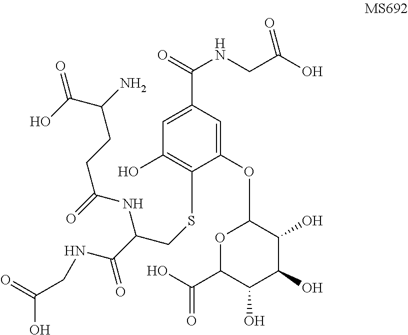 [SMILES: NC(CCC(=O)NC(CSc1c(O)cc(C(=O)NCC(=O)O)cc1OC1OC(C(=O)O)[C@@H](O)[C@H](O)[C@H]1O)C(=O)NCC(=O)O)C(=O)O]

While not being limited to any one particular mechanism, the following provides an example for MS292 metabolism via a nitroreductase or glutathione conjugation mechanism:

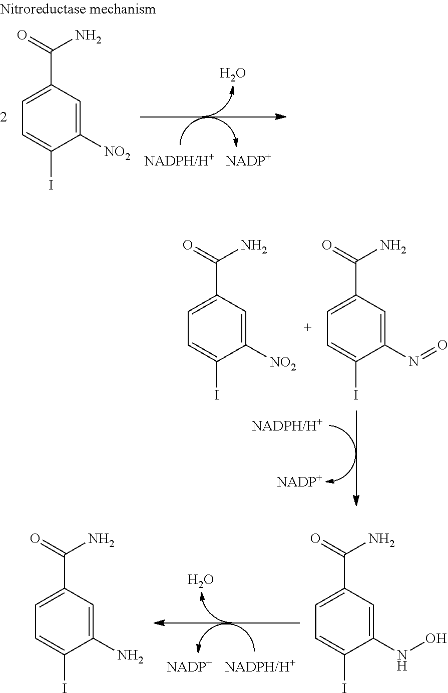

4-iodo-3-nitrobenzamide glutathione conjugation and metabolism:

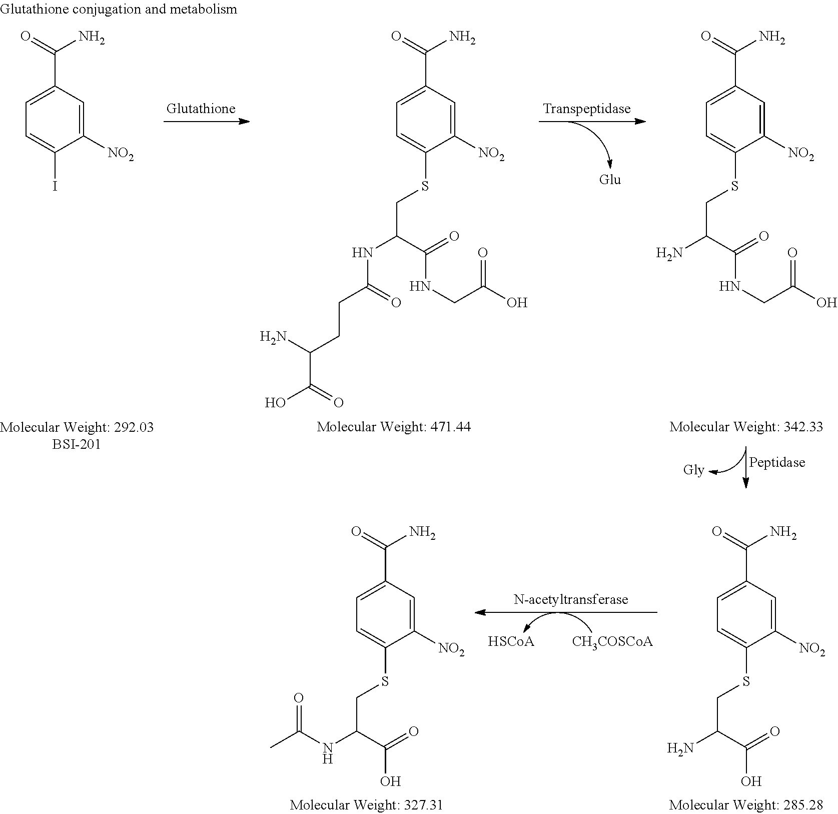

Any one of the metabolites of 4-iodo-3-nitrobenzamide described herein may be used in any one of the methods provided herein. Metabolites of 4-iodo-3-nitrobenzamide include, for example, 4-iodo-3-aminobenzoic acid (“IABA”), 4-iodo-3-aminobenzamide (“IABM”), 4-iodo-3-nitrosobenzamide (“BNO”), and 4-iodo-3-hydroxyaminobenzamide (“BNHOH”). Metabolites and methods of making metabolites are disclosed in U.S. Publication No. 2008/0103104 and U.S. Pat. No. 5,877,185, which are hereby incorporated by reference in their entirety, and in particular with respect to the metabolites and methods of making metabolites.

In some embodiments, 4-iodo-3-nitrobenzamide or a metabolite thereof or a pharmaceutically acceptable salt thereof is administered. In some embodiments, 4-iodo-3-nitrobenzamide or a pharmaceutically acceptable salt thereof is administered. In some embodiments, a metabolite of 4-iodo-3-nitrobenzamide is administered. In some embodiments, the metabolite of 4-iodo-3-nitrobenzamide is 4-iodo-3-aminobenzoic acid or 4-iodo-3-aminobenzamide.

The dosage range for the metabolites described herein used for treating breast cancer described herein may be in the range of about 0.0004 to about 0.5 mmol/kg (millimoles of metabolite per kilogram of patient's body weight), which dosage corresponds, on a molar basis, to a range of about 0.1 to about 100 mg/kg of 4-iodo-3-nitrobenzamide. Other effective ranges of dosages for metabolites are 0.0024-0.5 mmol/kg and 0.0048-0.25 mmol/kg. Such doses may be administered on a daily, every-other-daily, twice-weekly, weekly, bi-weekly, monthly or other suitable schedule. Essentially the same modes of administration may be employed for the metabolites as for 4-iodo-3-nitrobenzamide—e.g., oral, i.v., i.p., etc.

In some embodiments, 4-iodo-3-nitrobenzamide or a pharmaceutically acceptable salt thereof is administered. In some embodiments, a metabolite of 4-iodo-3-nitrobenzamide or a pharmaceutically acceptable salt of a metabolite of 4-iodo-3-nitrobenzamide is administered. The term “pharmaceutically acceptable salt” means those salts which retain the biological effectiveness and properties of the compounds used herein, and which are not biologically or otherwise undesirable. For example, a pharmaceutically acceptable salt does not interfere with the beneficial effect of the compound described herein in treating breast cancer.

Typical salts are those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, where compounds contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine. In some embodiments, 4-iodo-3-nitrobenzamide is formulated in 25% (w/v) hydroxypropyl-β-cyclodextrin and 10 mM phosphate buffer for intravenous administration as described in U.S. Patent Publication No. 2010/0160442, which is incorporated herein by reference.

### Irinotecan

Irinotecan is a topoisomerase 1 inhibitor. Irinotecan ((S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate), also known as CPT-11, has the following structure:

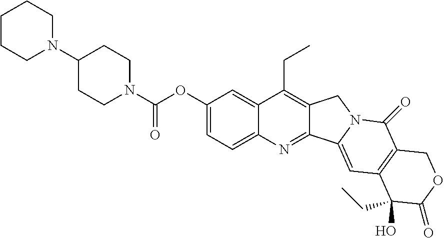 [SMILES: CCc1c2c(nc3ccc(OC(=O)N4CCC(N5CCCCC5)CC4)cc13)-c1cc3c(c(=O)n1C2)COC(=O)[C@]3(O)CC]

Irinotecan is available, for example, from Pfizer under the trade name Camptosar®. In some embodiments, irinotecan or a pharmaceutically acceptable salt thereof is used in combination with 4-iodo-3-nitrobenzamide, or a metabolite thereof, or a pharmaceutically acceptable salt or solvate thereof, for treating breast cancer (e.g., locally advanced or metastatic breast cancer). Methods of making irinotecan are known to one skilled in the art.

In some embodiments, 4-iodo-3-nitrobenzamide, or a metabolite thereof, or a pharmaceutically acceptable salt or solvate thereof potentiates the effect of irinotecan. In some embodiments, the amount of irinotecan administered to treat the breast cancer (e.g., metastatic breast cancer) is lowered when irinotecan is administered in combination with 4-iodo-3-nitrobenzamide, or a metabolite thereof, or a pharmaceutically acceptable salt or solvate, compared to the amount of irinotecan administered to treat the breast cancer (e.g., metastatic breast cancer) when irinotecan is administered not in combination with 4-iodo-3-nitrobenzamide, or a metabolite thereof, or a pharmaceutically acceptable salt or solvate. In some embodiments, the toxicity is reduced when irinotecan is administered in combination with 4-iodo-3-nitrobenzamide, or a metabolite thereof, or a pharmaceutically acceptable salt or solvate, compared to the toxicity when irinotecan is administered not in combination with 4-iodo-3-nitrobenzamide, or a metabolite thereof, or a pharmaceutically acceptable salt or solvate.

While not wishing to be bound by theory, it is thought that combined treatment of 4-iodo-3-nitrobenzamide with irinotecan described herein may permit efficacious dosing of irinotecan at a lower, and hence less toxic, dose. In some embodiments, the effective dose of irinotecan used with 4-iodo-3-nitrobenzamide may be about 10 to about 90%, about 10 to about 80%, about 10 to about 60%, about 10 to about 50%, less than about 90%, less than about 80%, less than about 60%, less than about 50% or less than about 40% of an effective dose of irinotecan used without administration of 4-iodo-3-nitrobenzamide.

The dosage of irinotecan used in the present invention may vary depending upon the patient's age, height, weight, overall health, etc. In some embodiments, the dosage of irinotecan is in the range of about 10 mg/m² to about 1000 mg/m², about 25 mg/m² to about 500 mg/m², about 50 mg/m² to about 200 mg/m², about 75 mg/m² to about 200 mg/m², about 75 mg/m² to about 150 mg/m², or about 80 mg/m² to about 125 mg/m². In some embodiments, the dosage of irinotecan is greater than or at least about any of 25 mg/m², 50 mg/m², 75 mg/m², 80 mg/m², 100 mg/m², 125 mg/m², 150 mg/m², 175 mg/m², 200 mg/m², 250 mg/m², or 300 mg/m². In some embodiments, the dosage of irinotecan is about any of 50 mg/m², 75 mg/m², 80 mg/m², 100 mg/m², 125 mg/m², 150 mg/m², 175 mg/m², 200 mg/m², 250 mg/m², or 300 mg/m². Irinotecan may be administered intravenously, e.g. by IV infusion over about 10 to about 500 minutes, about 10 to about 300 minutes, about 30 to about 180 minutes, about 45 to about 120 minutes, about 60 minutes (i.e. about 1 hour), or about 90 minutes. In some embodiments, irinotecan may alternatively be administered orally.

### Combination Therapy

In some embodiments, a method provided herein may further comprise another anti-cancer therapy including but not limited to surgery, radiation therapy (e.g., X ray), chemotherapy (such as anti-tumor agent), gene therapy, immunotherapy, DNA therapy, viral therapy, adjuvant therapy, immunotherapy, neoadjuvant therapy, RNA therapy, nanotherapy, or a combination thereof. In some embodiments, the radiation therapy comprises administering to the subject or patient gamma irradiation.

Provided herein are 4-iodo-3-nitrobenzamide or a pharmaceutically acceptable salt or solvate, or a metabolite thereof described herein and irinotecan or a pharmaceutically acceptable salt thereof, in combination with one or more additional therapy or therapies used in the treatment of the breast cancer (e.g., metastatic breast cancer). The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PI3k/mTOR pathway, HSP90 inhibitor, tubulin inhibitor, apoptosis inhibitor, and/or chemopreventative agent.

Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, by a significant period of time.

Combination therapy may be conducted at any suitable time so long as a beneficial effect from the co-action of the combination of selected additional therapy(ies) is achieved. The additional therapy may be administered before, after, or at the same time as the administration of 4-iodo-3-nitrobenazmide (or pharmaceutically acceptable salt or solvate thereof, or metabolite thereof) and irinotecan. “At the same time” means that the additional therapy is administered approximately at the same time as the administration of the 4-iodo-3-nitrobenazmide (or pharmaceutically acceptable salt or solvate thereof, or metabolite thereof) and irinotecan, e.g., within several hours before or after the administration of one or both of 4-iodo-3-nitrobenazmide or irinotecan.

Combination therapy may be conducted as a sequential administration or a concurrent administration. Sequential administration in this context means that the additional therapy(ies) and the administration of 4-iodo-3-nitrobenazmide (or pharmaceutically acceptable salt or solvate thereof, or metabolite thereof) and irinotecan are administered with a time separation of more than about 15 minutes, such as more than about any of 20, 30, 40, 50, 60, or more minutes. Either the additional therapy(ies) may be administered first, or the administration of 4-iodo-3-nitrobenazmide (or pharmaceutically acceptable salt or solvate thereof, or metabolite thereof) and irinotecan may be administered first. The additional therapy(ies), 4-iodo-3-nitrobenazmide (or pharmaceutically acceptable salt or solvate thereof, or metabolite thereof), and irinotecan are contained in separate compositions, which may be contained in the same or different packages or kits. Concurrent administration in this context means that the administration of the additional therapy(ies) and the administration of 4-iodo-3-nitrobenazmide (or pharmaceutically acceptable salt or solvate thereof, or metabolite thereof) and irinotecan overlap with each other.

It will be appreciated that when using a combination of 4-iodo-3-nitrobenazmide (or pharmaceutically acceptable salt or solvate thereof, or metabolite thereof) and irinotecan and an additional chemotherapeutic or other agent, the one ore more of the 4-iodo-3-nitrobenazmide (or pharmaceutically acceptable salt or solvate thereof, or metabolite thereof), irinotecan and the other pharmacologically active agent may be in the same pharmaceutically acceptable carrier and therefore administered simultaneously.

In some cases, the beneficial effect is achieved when the additional therapy is temporally removed from the administration of the 4-iodo-3-nitrobenazmide (or pharmaceutically acceptable salt or solvate thereof, or metabolite thereof) and irinotecan, by a significant period of time (e.g., about 12 hours, about 24 hours, about 36 hours, about 48 hours, etc.), or, for example, spaced apart by at least 1 day, 2 days, 3 days, 4 days, 5 days, 6 days, 7 days, 8 days, 9 days, 10 days, etc. For example, administration on different days of a treatment cycle, such as the treatment cycles described herein. The interval between administration of the 4-iodo-3-nitrobenzamide, irinotecan and/or additional agents or therapies may vary within a treatment cycle (e.g., administration is not always spaced apart by 1 day, but may be intervals of 1 days followed by an interval of 3 days, etc.). Similarly, at certain times during the treatment cycle, 4-iodo-3-nitrobenzamide, irinotecan and/or additional agents or therapies may be administered at the same time, and at other points during the treatment administered at different times.

### Anti-Tumor Agents

In some embodiments, a method provided herein may further comprise at least one anti-tumor agent. For example, a method provided herein comprising administering (a) 4-iodo-3-nitrobenzamide or metabolite thereof or a pharmaceutically acceptable salt thereof, (b) irinotecan or a pharmaceutically acceptable salt thereof may further comprise at least one anti-tumor agent.

Anti-tumor agents that may be used in the present invention include but are not limited to antitumor alkylating agents, antitumor antimetabolites, antitumor antibiotics, plant-derived antitumor agents, antitumor platinum-complex compounds, antitumor camptothecin derivatives, antitumor tyrosine kinase inhibitors, anti-tumor viral agent, monoclonal antibodies, interferons, biological response modifiers, and other agents that exhibit anti-tumor activities, or a pharmaceutically acceptable salt thereof.

In some embodiments, the anti-tumor agent is an alkylating agent. The term “alkylating agent” herein generally refers to an agent giving an alkyl group in the alkylation reaction in which a hydrogen atom of an organic compound is substituted with an alkyl group. Examples of anti-tumor alkylating agents include but are not limited to nitrogen mustard N-oxide, cyclophosphamide, ifosfamide, melphalan, busulfan, mitobronitol, carboquone, thiotepa, ranimustine, nimustine, temozolomide or carmustine.

In some embodiments, the anti-tumor agent is an antimetabolite. The term “antimetabolite” used herein includes, in a broad sense, substances which disturb normal metabolism and substances which inhibit the electron transfer system to prevent the production of energy-rich intermediates, due to their structural or functional similarities to metabolites that are important for living organisms (such as vitamins, coenzymes, amino acids and saccharides). Examples of antimetabolites that have anti-tumor activities include but are not limited to methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine or pemetrexed disodium.

In some embodiments, the anti-tumor agent is an antitumor antibiotic. Examples of antitumor antibiotics include but are not limited to actinomycin D, doxorubicin, daunorubicin, neocarzinostatin, bleomycin, peplomycin, mitomycin C, aclarubicin, pirarubicin, epirubicin, zinostatin, stimalamer, idarubicin, sirolimus or valrubicin.

In some embodiments, the anti-tumor agent is a plant-derived antitumor agent. Examples of plant-derived antitumor agents include but are not limited to vincristine, vinblastine, vindesine, etoposide, sobuzoxane, docetaxel, paclitaxel and vinorelbine.

In some embodiments, the anti-tumor agent is a camptothecin derivative that exhibits anti-tumor activities. Examples of anti-tumor camptothecin derivatives include but are not limited to camptothecin, 10-hydroxycamptothecin, topotecan, irinotecan or 9-aminocamptothecin. Irinotecan may be metabolized in vivo and exhibit antitumor effect as SN-38. The action mechanism and the activity of the camptothecin derivatives are believed to be virtually the same as those of camptothecin (e.g., Nitta et al., Gan to Kagaku Ryoho, 14, 850-857 (1987)).

In some embodiments, the anti-tumor agent is an organoplatinum compound or a platinum coordination compound having antitumor activity. The terms “organoplatinum compound,” “platinum compound,” or “platinum complex” and the like as used herein refer to a platinum-containing compound which provides platinum in ion form. Organoplatinum compounds include but are not limited to cisplatin; cis-diamminediaquoplatinum (II)-ion; chloro(diethylenetriamine)-platinum (II) chloride; dichloro(ethylenediamine)-platinum (II); diammine(1,1-cyclobutanedicarboxylato) platinum (II) (carboplatin); spiroplatin; iproplatin; diammine(2-ethylmalonato)platinum (II); ethylenediaminemalonatoplatinum (II); aqua(1,2-diaminodicyclohexane)sulfatoplatinum (II); aqua(1,2-diaminodicyclohexane)malonatoplatinum (II); (1,2-diaminocyclohexane)malonatoplatinum (II); (4-carboxyphthalato)(1,2-diaminocyclohexane) platinum (II); (1,2-diaminocyclohexane)-(isocitrato)platinum (II); (1,2-diaminocyclohexane)oxalatoplatinum (II); ormaplatin; tetraplatin; carboplatin, nedaplatin and oxaliplatin. Further, other antitumor organoplatinum compounds mentioned in the specification are known and are commercially available and/or producible by a person having ordinary skill in the art by conventional techniques.

In some embodiments, the anti-tumor agent is an antitumor tyrosine kinase inhibitor. The term “tyrosine kinase inhibitor” herein refers to a chemical substance inhibiting “tyrosine kinase” which transfers a λ-phosphate group of ATP to a hydroxyl group of a specific tyrosine in protein. Examples of anti-tumor tyrosine kinase inhibitors include but are not limited to gefitinib, imatinib, erlotinib, Sutent, Nexavar, Recentin, ABT-869, and Axitinib.

In some embodiments, the anti-tumor agent is an antibody or a binding portion of an antibody that exhibits anti-tumor activity. In some embodiments, the anti-tumor agent is a monoclonal antibody. Examples thereof include but are not limited to abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, trastuzumab, or any antibody fragments specific for antigens.

In some embodiments, the anti-tumor agent is an interferon. Such interferon has antitumor activity, and it is a glycoprotein which is produced and secreted by most animal cells upon viral infection. It has not only the effect of inhibiting viral growth but also various immune effector mechanisms including inhibition of growth of cells (in particular, tumor cells) and enhancement of the natural killer cell activity, thus being designated as one type of cytokine. Examples of anti-tumor interferons include but are not limited to interferon α, interferon α-2a, interferon α-2b, interferon β, interferon γ-1a and interferon γ-n1.

In some embodiments, the anti-tumor agent is a biological response modifier. It is generally the generic term for substances or drugs for modifying the defense mechanisms of living organisms or biological responses such as survival, growth or differentiation of tissue cells in order to direct them to be useful for an individual against tumor, infection or other diseases. Examples of the biological response modifier include but are not limited to krestin, lentinan, sizofiran, picibanil and ubenimex.

In some embodiments, the anti-tumor agents include but are not limited to mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

The above-described terms “antitumor alkylating agent”, “antitumor antimetabolite”, “antitumor antibiotic”, “plant-derived antitumor agent”, “antitumor platinum coordination compound”, “antitumor camptothecin derivative”, “antitumor tyrosine kinase inhibitor”, “monoclonal antibody”, “interferon”, “biological response modifier” and “other antitumor agent” are all known and are either commercially available or producible by a person skilled in the art by methods known per se or by well-known or conventional methods. The process for preparation of gefitinib is described, for example, in U.S. Pat. No. 5,770,599; the process for preparation of cetuximab is described, for example, in WO 96/40210; the process for preparation of bevacizumab is described, for example, in WO 94/10202; the process for preparation of oxaliplatin is described, for example, in U.S. Pat. Nos. 5,420,319 and 5,959,133; the process for preparation of gemcitabine is described, for example, in U.S. Pat. Nos. 5,434,254 and 5,223,608; and the process for preparation of camptothecin is described in U.S. Pat. Nos. 5,162,532, 5,247,089, 5,191,082, 5,200,524, 5,243,050 and 5,321,140; the process for preparation of irinotecan is described, for example, in U.S. Pat. No. 4,604,463; the process for preparation of topotecan is described, for example, in U.S. Pat. No. 5,734,056; the process for preparation of temozolomide is described, for example, in JP-B No. 4-5029; and the process for preparation of rituximab is described, for example, in JP-W No. 2-503143.

The above-mentioned antitumor alkylating agents are commercially available, as exemplified by the following: nitrogen mustard N-oxide from Mitsubishi Pharma Corp. as Nitrorin (tradename); cyclophosphamide from Shionogi & Co., Ltd. as Endoxan (tradename); ifosfamide from Shionogi & Co., Ltd. as Ifomide (tradename); melphalan from GlaxoSmithKline Corp. as Alkeran (tradename); busulfan from Takeda Pharmaceutical Co., Ltd. as Mablin (tradename); mitobronitol from Kyorin Pharmaceutical Co., Ltd. as Myebrol (tradename); carboquone from Sankyo Co., Ltd. as Esquinon (tradename); thiotepa from Sumitomo Pharmaceutical Co., Ltd. as Tespamin (tradename); ranimustine from Mitsubishi Pharma Corp. as Cymerin (tradename); nimustine from Sankyo Co., Ltd. as Nidran (tradename); temozolomide from Schering Corp. as Temodar (tradename); and carmustine from Guilford Pharmaceuticals Inc. as Gliadel Wafer (tradename).

The above-mentioned antitumor antimetabolites are commercially available, as exemplified by the following: methotrexate from Takeda Pharmaceutical Co., Ltd. as Methotrexate (tradename); 6-mercaptopurine riboside from Aventis Corp. as Thioinosine (tradename); mercaptopurine from Takeda Pharmaceutical Co., Ltd. as Leukerin (tradename); 5-fluorouracil from Kyowa Hakko Kogyo Co., Ltd. as 5-FU (tradename); tegafur from Taiho Pharmaceutical Co., Ltd. as Futraful (tradename); doxyfluridine from Nippon Roche Co., Ltd. as Furutulon (tradename); carmofur from Yamanouchi Pharmaceutical Co., Ltd. as Yamafur (tradename); cytarabine from Nippon Shinyaku Co., Ltd. as Cylocide (tradename); cytarabine ocfosfate from Nippon Kayaku Co., Ltd. as Strasid (tradename); enocitabine from Asahi Kasei Corp. as Sanrabin (tradename); S-1 from Taiho Pharmaceutical Co., Ltd. as TS-1 (tradename); gemcitabine from Eli Lilly & Co. as Gemzar (tradename); fludarabine from Nippon Schering Co., Ltd. as Fludara (tradename); and pemetrexed disodium from Eli Lilly & Co. as Alimta (tradename).

The above-mentioned antitumor antibiotics are commercially available, as exemplified by the following: actinomycin D from Banyu Pharmaceutical Co., Ltd. as Cosmegen (tradename); doxorubicin from Kyowa Hakko Kogyo Co., Ltd. as adriacin (tradename); daunorubicin from Meiji Seika Kaisha Ltd. as Daunomycin; neocarzinostatin from Yamanouchi Pharmaceutical Co., Ltd. as Neocarzinostatin (tradename); bleomycin from Nippon Kayaku Co., Ltd. as Bleo (tradename); pepromycin from Nippon Kayaku Co, Ltd. as Pepro (tradename); mitomycin C from Kyowa Hakko Kogyo Co., Ltd. as Mitomycin (tradename); aclarubicin from Yamanouchi Pharmaceutical Co., Ltd. as Aclacinon (tradename); pirarubicin from Nippon Kayaku Co., Ltd. as Pinorubicin (tradename); epirubicin from Pharmacia Corp. as Pharmorubicin (tradename); zinostatin stimalamer from Yamanouchi Pharmaceutical Co., Ltd. as Smancs (tradename); idarubicin from Pharmacia Corp. as Idamycin (tradename); sirolimus from Wyeth Corp. as Rapamune (tradename); and valrubicin from Anthra Pharmaceuticals Inc. as Valstar (tradename).

The above-mentioned plant-derived antitumor agents are commercially available, as exemplified by the following: vincristine from Shionogi & Co., Ltd. as Oncovin (tradename); vinblastine from Kyorin Pharmaceutical Co., Ltd. as Vinblastine (tradename); vindesine from Shionogi & Co., Ltd. as Fildesin (tradename); etoposide from Nippon Kayaku Co., Ltd. as Lastet (tradename); sobuzoxane from Zenyaku Kogyo Co., Ltd. as Perazolin (tradename); docetaxel from Aventis Corp. as Taxsotere (tradename); paclitaxel from Bristol-Myers Squibb Co. as Taxol (tradename); and vinorelbine from Kyowa Hakko Kogyo Co., Ltd. as Navelbine (tradename).

The above-mentioned antitumor platinum coordination compounds are commercially available, as exemplified by the following: cisplatin from Nippon Kayaku Co., Ltd. as Randa (tradename); carboplatin from Bristol-Myers Squibb Co. as Paraplatin (tradename); nedaplatin from Shionogi & Co., Ltd. as Aqupla (tradename); and oxaliplatin from Sanofi-Synthelabo Co. as Eloxatin (tradename).

The above-mentioned antitumor camptothecin derivatives are commercially available, as exemplified by the following: irinotecan from Yakult Honsha Co., Ltd. as Campto (tradename); topotecan from GlaxoSmithKline Corp. as Hycamtin (tradename); and camptothecin from Aldrich Chemical Co., Inc., U.S.A.

The above-mentioned antitumor tyrosine kinase inhibitors are commercially available, as exemplified by the following: gefitinib from AstraZeneca Corp. as Iressa (tradename); imatinib from Novartis AG as Gleevec (tradename); and erlotinib from OSI Pharmaceuticals Inc. as Tarceva (tradename).

The above-mentioned monoclonal antibodies are commercially available, as exemplified by the following: cetuximab from Bristol-Myers Squibb Co. as Erbitux (tradename); bevacizumab from Genentech, Inc. as Avastin (tradename); rituximab from Biogen Idec Inc. as Rituxan (tradename); alemtuzumab from Berlex Inc. as Campath (tradename); and trastuzumab from Chugai Pharmaceutical Co., Ltd. as Herceptin (tradename).

The above-mentioned interferons are commercially available, as exemplified by the following: interferon a from Sumitomo Pharmaceutical Co., Ltd. as Sumiferon (tradename); interferon α-2a from Takeda Pharmaceutical Co., Ltd. as Canferon-A (tradename); interferon α-2b from Schering-Plough Corp. as Intron A (tradename); interferon β from Mochida Pharmaceutical Co., Ltd. as IFN.beta. (tradename); interferon γ-1a from Shionogi & Co., Ltd. as Immunomax-γ (tradename); and interferon γ-n1 from Otsuka Pharmaceutical Co., Ltd. as Ogamma (tradename).

The above-mentioned biological response modifiers are commercially available, as exemplified by the following: krestin from Sankyo Co., Ltd. as krestin (tradename); lentinan from Aventis Corp. as Lentinan (tradename); sizofiran from Kaken Seiyaku Co., Ltd. as Sonifiran (tradename); picibanil from Chugai Pharmaceutical Co., Ltd. as Picibanil (tradename); and ubenimex from Nippon Kayaku Co., Ltd. as Bestatin (tradename).

The above-mentioned other antitumor agents are commercially available, as exemplified by the following: mitoxantrone from Wyeth Lederle Japan, Ltd. as Novantrone (tradename); L-asparaginase from Kyowa Hakko Kogyo Co., Ltd. as Leunase (tradename); procarbazine from Nippon Roche Co., Ltd. as Natulan (tradename); dacarbazine from Kyowa Hakko Kogyo Co., Ltd. as Dacarbazine (tradename); hydroxycarbamide from Bristol-Myers Squibb Co. as Hydrea (tradename); pentostatin from Kagaku Oyobi Kessei Ryoho Kenkyusho as Coforin (tradename); tretinoin from Nippon Roche Co., Ltd. As Vesanoid (tradename); alefacept from Biogen Idec Inc. as Amevive (tradename); darbepoetin alfa from Amgen Inc. as Aranesp (tradename); anastrozole from AstraZeneca Corp. as Arimidex (tradename); exemestane from Pfizer Inc. as Aromasin (tradename); bicalutamide from AstraZeneca Corp. as Casodex (tradename); leuprorelin from Takeda Pharmaceutical Co., Ltd. as Leuplin (tradename); flutamide from Schering-Plough Corp. as Eulexin (tradename); fulvestrant from AstraZeneca Corp. as Faslodex (tradename); pegaptanib octasodium from Gilead Sciences, Inc. as Macugen (tradename); denileukin diftitox from Ligand Pharmaceuticals Inc. as Ontak (tradename); aldesleukin from Chiron Corp. as Proleukin (tradename); thyrotropin alfa from Genzyme Corp. as Thyrogen (tradename); arsenic trioxide from Cell Therapeutics, Inc. as Trisenox (tradename); bortezomib from Millennium Pharmaceuticals, Inc. as Velcade (tradename); capecitabine from Hoffmann-La Roche, Ltd. as Xeloda (tradename); and goserelin from AstraZeneca Corp. as Zoladex (tradename). The term “antitumor agent” as used in the specification includes the above-described antitumor alkylating agent, antitumor antimetabolite, antitumor antibiotic, plant-derived antitumor agent, antitumor platinum coordination compound, antitumor camptothecin derivative, antitumor tyrosine kinase inhibitor, monoclonal antibody, interferon, biological response modifier, and other antitumor agents.

Other anti-tumor agents or anti-neoplastic agents can also be used. Such suitable anti-tumor agents or anti-neoplastic agents include, but are not limited to, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane, Actinomycin-D, Adriamycin, Adrucil, Agrylin, Ala-Cort, Aldesleukin, Alemtuzumab, ALIMTA, Alitretinoin, Alkaban-AQ, Alkeran, All-transretinoic Acid, Alpha Interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arranon, Arsenic Trioxide, Asparaginase, ATRA, Avastin, Azacitidine, BCG, BCNU, Bendamustine, Bevacizumab, Bexarotene, BEXXAR, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine Wafer, Casodex, CC-5013, CCI-779, CCNU, CDDP, CeeNU, Cerubidine, Cetuximab, Chlorambucil, Cisplatin, Citrovorum Factor, Cladribine, Cortisone, Cosmegen, CPT-11, Cyclophosphamide, Cytadren, Cytarabine, Cytarabine Liposomal, Cytosar-U, Cytoxan, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome, Decadron, Decitabine, Delta-Cortef, Deltasone, Denileukin Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil, Doxorubicin, Doxorubicin Liposomal, Droxia™, DTIC, DTIC-Dome, Duralone, Efudex, Eligard, Ellence, Eloxatin, Elspar, Emcyt, Epirubicin, Epoetin Alfa, Erbitux, Erlotinib, Erwinia L-asparaginase, Estramustine, Ethyol, Etopophos, Etoposide, Etoposide Phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar & Gemzar Side Effects—Chemotherapy Drugs, Gleevec, Gliadel Wafer, GM-CSF, Goserelin, Granulocyte—Colony Stimulating Factor, Granulocyte Macrophage Colony Stimulating Factor, Halotestin, Herceptin, Hexadrol, Hexylen, Hexamethylmelamine, HMM, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin, Idarubicin, Ifex, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A (interferon alfa-2b), Iressa, Irinotecan, Isotretinoin, Ixabepilone, Ixempra, Kidrolase (t), Lanacort, Lapatinib, L-asparaginase, LCR, Lenalidomide, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine Hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mustargen, Mustine, Mutamycin, Myleran, Mylocel, Mylotarg, Navelbine, Nelarabine, Neosar, Neulasta, Neumega, Neupogen, Nexavar, Nilandron, Nilutamide, Nipent, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Paclitaxel Protein-bound, Pamidronate, Panitumumab, Panretin, Paraplatin, Pediapred, PEG Interferon, Pegaspargase, Pegfilgrastim, PEG-INTRON, PEG-L-asparaginase, PEMETREXED, Pentostatin, Phenylalanine Mustard, Platinol, Platinol-AQ, Prednisolone, Prednisone, Prelone, Procarbazine, PROCRIT, Proleukin, Prolifeprospan 20 with Carmustine Implant, Purinethol, Raloxifene, Revlimid, Rheumatrex, Rituxan, Rituximab, Roferon-A (Interferon Alfa-2a), Rubex, Rubidomycin hydrochloride, Sandostatin, Sandostatin LAR, Sargramostim, Solu-Cortef, Solu-Medrol, Sorafenib, SPRYCEL, STI-571, Streptozocin, SU11248, Sunitinib, Sutent, Tamoxifen, Tarceva, Targretin, Taxol, Taxotere, Temodar, Temozolomide, Temsirolimus, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Torisel, Tositumomab, Trastuzumab, Tretinoin, Trexall™, Trisenox, TSPA, TYKERB, VCR, Vectibix, Vectibix, Velban, Velcade, VePesid, Vesanoid, Viadur, Vidaza, Vinblastine, Vinblastine Sulfate, Vincasar Pfs, Vincristine, Vinorelbine, Vinorelbine tartrate, VLB, VM-26, Vorinostat, VP-16, Vumon, Xeloda, Zanosar, Zevalin, Zinecard, Zoladex, Zoledronic acid, Zolinza, Zometa.

In some embodiments, the anti-tumor agent is administered prior to, concomitant with or subsequent to administering the effective amount of 4-iodo-3-nitrobenzamide or irinotecan.

In some embodiments, a method provided herein further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

Anti-tumor agents and therapies are further described below.

### Alkylating Agents

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with any of the alkylating agents described herein.

Alkylating agents are known to act through the alkylation of macromolecules such as the DNA of cancer cells, and are usually strong electrophiles. This activity can disrupt DNA synthesis and cell division. Examples of alkylating reagents suitable for use herein include nitrogen mustards and their analogues and derivatives including, cyclophosphamide, ifosfamide, chlorambucil, estramustine, mechlorethamine hydrochloride, melphalan, and uracil mustard. Other examples of alkylating agents include alkyl sulfonates (e.g. busulfan), nitrosoureas (e.g. carmustine, lomustine, and streptozocin), triazenes (e.g. dacarbazine and temozolomide), ethylenimines/methylmelamines (e.g. altretamine and thiotepa), and methylhydrazine derivatives (e.g. procarbazine). Included in the alkylating agent group are the alkylating-like platinum-containing drugs comprising carboplatin, cisplatin, and oxaliplatin.

### Topoisomerase Inhibitors

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with a second topoisomerase inhibitor, for example, topotecan.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. Topoisomerases have become popular targets for cancer chemotherapy treatments. It is thought that topoisomerase inhibitors block the ligation step of the cell cycle, generating single and double stranded breaks that harm the integrity of the genome. Introduction of these breaks subsequently lead to apoptosis and cell death. Topoisomerase inhibitors are often divided according to which type of enzyme it inhibits. Topoisomerase I, the type of topoisomerase most often found in eukaryotes, is targeted by topotecan, irinotecan, lurtotecan and exatecan, each of which is commercially available. Topotecan is available from GlaxoSmithKline under the trade name Hycamtim®. Irinotecan is available from Pfizer under the trade name Camptosar®. Lurtotecan may be obtained as a liposomal formulation from Gilead Sciences Inc.

Compounds that target type II topoisomerase are split into two main classes: topoisomerase poisons, which target the topoisomerase-DNA complex, and topoisomerase inhibitors, which disrupt catalytic turnover. Topo II poisons include but are not limited to eukaryotic type II topoisomerase inhibitors (topo II): amsacrine, etoposide, etoposide phosphate, teniposide and doxorubicin. These drugs are anti-cancer therapies. Examples of topoisomerase inhibitors include ICRF-193. These inhibitors target the N-terminal ATPase domain of topo II and prevent topo II from turning over. The structure of this compound bound to the ATPase domain has been solved by Classen (Proceedings of the National Academy of Science, 2004) showing that the drug binds in a non-competitive manner and locks down the dimerization of the ATPase domain.

### Antimetabolites

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with any of the antimetabolites described herein. Antimetabolites are drugs that interfere with normal cellular metabolic processes. Since cancer cells are rapidly replicating, interference with cellular metabolism affects cancer cells to a greater extent than host cells.

### Platinum Complexes

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with any of the platinum complexes described herein. Platinum complexes are pharmaceutical agents or pharmaceutical compositions used to treat cancer, which contain at least one platinum center complexed with at least one organic group.

### Taxanes

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with any of the taxanes described herein.

Taxanes are drugs that are derived from the twigs, needles and bark of Pacific yew tress, Taxus brevifolia. In particular paclitaxel may be derived from 10-deacetylbaccatin through known synthetic methods. Taxanes such as paclitaxel and its derivative docetaxel have demonstrated antitumor activity in a variety of tumor types. The taxanes interfere with normal function of microtubule growth by hyperstabilizing their structure, thereby destroying the cell's ability to use its cytoskeleton in a normal manner. Specifically, the taxanes bind to the β subunit of tubulin, which is the building block of microtubules. The resulting taxane/tubulin complex cannot disassemble, which results in aberrant cell function and eventual cell death. Paclitaxel induces programmed cell death (apoptosis) in cancer cells by binding to an apoptosis-inhibiting protein called Bcl-2 (B-cell leukemia 2), thereby preventing Bcl-2 from inhibiting apoptosis.

Thus paclitaxel has proven to be an effective treatment for various cancers, as it down-regulates cell division by interrupting normal cytoskeletal rearrangement during cell division and it induces apoptosis via the anti-Bcl-2 mechanism.

### Anti-Angiogenic Agents

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with any of the anti-angiogenic agents described herein.

An angiogenesis inhibitor is a substance that inhibits angiogenesis (the growth of new blood vessels). Every solid tumor (in contrast to leukemia) needs to generate blood vessels to keep it alive once it reaches a certain size. Tumors can grow only if they form new blood vessels. Usually, blood vessels are not built elsewhere in an adult body unless tissue repair is actively in process. The angiostatic agent endostatin and related chemicals can suppress the building of blood vessels, preventing the cancer from growing indefinitely. In tests with patients, the tumor became inactive and stayed that way even after the endostatin treatment was finished. The treatment has very few side effects but appears to have very limited selectivity. Other angiostatic agents such as thalidomide and natural plant-based substances are being actively investigated.

Known inhibitors include the drug bevacizumab (Avastin), which binds vascular endothelial growth factor (VEGF), inhibiting its binding to the receptors that promote angiogenesis. Other anti-angiogenic agents include but are not limited to carboxyamidotriazole, TNF-470, CM101, IFN-alpha, IL-12, platelet factor-4, suramin, SU5416, thrombospondin, angiostatic steroids+heparin, cartilage-derived angiogenesis inhibitory factor, matrix metalloproteinase inhibitors, angiostatin, endostatin, 2-methoxyestradiol, tecogalan, thrombospondin, prolactin, α_{V}β₃ inhibitors and linomide.

### Her-2 Targeted Therapy

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with Herceptin.

Herceptin (trastuzumab) is a targeted therapy for use in early-stage HER2-positive breast cancers. Herceptin is approved for the adjuvant treatment of HER2-overexpressing, node-positive or node-negative (ER/PR-negative or with one high-risk feature) breast cancer. Herceptin can be used several different ways: as part of a treatment regimen including doxorubicin, cyclophosphamide, and either paclitaxel or docetaxel; with docetaxel and carboplatin; or as a single agent following multi-modality anthracycline-based therapy. Herceptin in combination with paclitaxel is approved for the first-line treatment of HER2-overexpressing metastatic breast cancer. Herceptin as a single agent is approved for treatment of HER2-overexpressing breast cancer in patients who have received one or more chemotherapy regimens for metastatic disease.

Lapatinib or lapatinib ditosylate is an orally active chemotherapeutic drug treatment for solid tumours such as breast cancer. During development it was known as small molecule GW572016. Patients who meet specific indication criteria may be prescribed lapatinib as part of combination therapy for breast cancer. Pharmacologically, lapatinib is a dual tyrosine kinase inhibitor that interrupts cancer-causing cellular signals. Lapatinib is used as a treatment for women's breast cancer in patients who have HER2-positive advanced breast cancer that has progressed after previous treatment with other chemotherapeutic agents, such as anthracycline, taxane-derived drugs, or trastuzumab (Herceptin, Genentech).

### Hormone Therapy

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with hormone therapy.

There are certain hormones that can attach to cancer cells and can affect their ability to multiply. The purpose of hormone therapy is to add, block or remove hormones. With breast cancer, the female hormones estrogen and progesterone can promote the growth of some breast cancer cells. So in these patients, hormone therapy is given to block the body's naturally occurring estrogen and fight the cancer's growth. There are two types of hormone therapy for breast cancer: drugs that inhibit estrogen and progesterone from promoting breast cancer cell growth and drugs or surgery to turn off the production of hormones from the ovaries.

Common hormone therapy drugs used for breast cancer include but are not limited to Tamoxifen, Fareston, Arimidex, Aromasin, Femara, and Zoladex.

### Tamoxifen-Hormone Antagonist

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with tamoxifen.

Tamoxifen (marketed as Nolvadex) decreases the chance that some early-stage breast cancers will recur and can prevent the development of cancer in the unaffected breast. Tamoxifen also slows or stops the growth of cancer cells present in the body. In addition, tamoxifen may offer an alternative to watchful waiting or prophylactic (preventative) mastectomy to women at high risk for developing breast cancer. Tamoxifen is a type of drug called a selective estrogen-receptor modulator (SERM). At the breast, it functions as an anti-estrogen. Estrogen promotes the growth of breast cancer cells and tamoxifen blocks estrogen from attaching to estrogen receptors on these cells. By doing this, it is believed that the growth of the breast cancer cells will be halted. Tamoxifen is often given along with chemotherapy and other breast cancer treatments. It is considered an option in the following cases: Treatment of ductal carcinoma in situ (DCIS) along with breast-sparing surgery or mastectomy; Adjuvant treatment of lobular carcinoma in situ (LCIS) to reduce the risk of developing more advanced breast cancer; Adjuvant treatment of metastatic breast cancer in men and women whose cancers are estrogen-receptor positive; Treatment of recurrent breast cancer; To prevent breast cancer in women at high risk for developing breast cancer.

### Steroidal and Non-Steroidal Aromatase Inhibitor

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with any of the aromatase inhibitors described herein.

Aromatase inhibitors (AI) are a class of drugs used in the treatment of breast cancer and ovarian cancer in postmenopausal women that block the aromatase enzyme. Aromatase inhibitors lower the amount of estrogen in post-menopausal women who have hormone-receptor-positive breast cancer. With less estrogen in the body, the hormone receptors receive fewer growth signals, and cancer growth can be slowed down or stopped.

Aromatase inhibitor medications include Arimidex (chemical name: anastrozole), Aromasin (chemical name: exemestane), and Femara (chemical name: letrozole). Each is taken by pill once a day, for up to five years. But for women with advanced (metastatic) disease, the medicine is continued as long as it is working well.

AIs are categorized into two types: irreversible steroidal inhibitors such as exemestane that form a permanent bond with the aromatase enzyme complex; and non-steroidal inhibitors (such as anastrozole, letrozole) that inhibit the enzyme by reversible competition.

Fulvestrant, also known as ICI-182,780, and “Faslodex” is a drug treatment of hormone receptor-positive metastatic breast cancer in postmenopausal women with disease progression following anti-estrogen therapy. It is an estrogen receptor antagonist with no agonist effects, which works both by down-regulating and by degrading the estrogen receptor. It is administered as a once-monthly injection.

### Targeted Therapy

Also provided are methods that comprise administering to a patient with breast cancer (e.g., locally advanced or metastatic breast cancer) an effective amount of 4-iodo-3-nitrobenzamide (or a metabolite thereof or a pharmaceutically acceptable salt thereof) and irinotecan (or a pharmaceutically acceptable salt thereof) in combination with an inhibitor targeting a growth factor receptor including but not limited to epidermal growth factor receptor (EGFR) and insulin-like growth factor 1 receptor (IGF1R).

EGFR is overexpressed in the cells of certain types of human carcinomas including but not limited to lung and breast cancers. Highly proliferating, invasive breast cancer cells often express abnormally high levels of the EGFR, and this is known to control both cell division and migration. The interest in EGFR is further enhanced by the availability and FDA approval of specific EGFR tyrosine kinase inhibitors, for example, Gefitinib Inhibition of EGFR is an important anti-cancer treatment. Examples of EGFR inhibitors include but are not limited to cetuximab, which is a chimeric monoclonal antibody given by intravenous injection for treatment of cancers including but not limited to metastatic colorectal cancer and head and neck cancer. Panitumimab is another example of EGFR inhibitor. It is a humanized monoclonal antibody against EGFR. Panitumimab has been shown to be beneficial and better than supportive care when used alone in patients with advanced colon cancer and is approved by the FDA for this use.

Activation of the type 1 insulin-like growth factor receptor (IGF1R) promotes proliferation and inhibits apoptosis in a variety of cell types. Transgenic mice expressing a constitutively active IGF1R or IGF-1 develop mammary tumors and increased levels of IGF1R have been detected in primary breast cancers (Yanochko et. al. Breast Cancer Research 2006). It has also been shown that the insulin-like growth factor 1 receptor (IGF1R) and HER2 display important signaling interactions in breast cancer. Specific inhibitors of one of these receptors may cross-inhibit the activity of the other. Targeting both receptors give the maximal inhibition of their downstream extracellular signal-regulated kinase 1/2 and AKT signaling pathways. Hence, such drug combinations may be clinically useful and may be beneficial even in tumors in which single drugs are inactive, as exemplified by the effect of the HER2/IGF1R inhibitor combination in HER2 nonoverexpressing MCF7 cells (Chakraborty A K, et. al, Cancer Res. 2008 Mar. 1; 68(5):1538-45). One example of an IGF1R inhibitor is CP-751871. CP-751871 is a human monoclonal antibody that selectively binds to IGF1R, preventing IGF1 from binding to the receptor and subsequent receptor autophosphorylation Inhibition of IGF1R autophosphorylation may result in a reduction in receptor expression on tumor cells that express IGF1R, a reduction in the anti-apoptotic effect of IGF, and inhibition of tumor growth. IGF1R is a receptor tyrosine kinase expressed on most tumor cells and is involved in mitogenesis, angiogenesis, and tumor cell survival.

### PI3K/mTOR Pathway

Phosphatidylinositol-3-kinase (PI3K) pathway deregulation is a common event in human cancer, either through inactivation of the tumor suppressor phosphatase and tensin homologue deleted from chromosome 10 or activating mutations of p110-α. These hotspot mutations result in oncogenic activity of the enzyme and contribute to therapeutic resistance to the anti-HER2 antibody trastuzumab. The PI3K pathway is, therefore, an attractive target for cancer therapy. NVP-BEZ235, a dual inhibitor of the PI3K and the downstream mammalian target of rapamycin (mTOR) has been shown to inhibit the activation of the downstream effectors Akt, S6 ribosomal protein, and 4EBP1 in breast cancer cells. NVP-BEZ235 inhibits the PI3K/mTOR axis and results in antiproliferative and antitumoral activity in cancer cells with both wild-type and mutated p110-α (Violeta Serra, et. al. Cancer Research 68, 8022-8030, Oct. 1, 2008).

### Hsp90 Inhibitors

These drugs target heat shock protein 90 (hsp90). Hsp90 is one of a class of chaperone proteins, whose normal job is to help other proteins acquire and maintain the shape required for those proteins to do their jobs. Chaperone proteins work by being in physical contact with other proteins. Hsp90 can also enable cancer cells to survive and even thrive despite genetic defects which would normally cause such cells to die. Thus, blocking the function of HSP90 and related chaperone proteins may cause cancer cells to die, especially if blocking chaperone function is combined with other strategies to block cancer cell survival.

### Tubulin Inhibitors

Tubulins are the proteins that form microtubules, which are key components of the cellular cytoskeleton (structural network). Microtubules are necessary for cell division (mitosis), cell structure, transport, signaling and motility. Given their primary role in mitosis, microtubules have been an important target for anticancer drugs—often referred to as antimitotic drugs, tubulin inhibitors and microtubule targeting agents. These compounds bind to tubulin in microtubules and prevent cancer cell proliferation by interfering with the microtubule formation required for cell division. This interference blocks the cell cycle sequence, leading to apoptosis.

### Apoptosis Inhibitors

The inhibitors of apoptosis (IAP) are a family of functionally- and structurally-related proteins, originally characterized in Baculovirus, which serve as endogenous inhibitors of apoptosis. The human IAP family consists of at least 6 members, and IAP homologs have been identified in numerous organisms. 10058-F4 is a c-Myc inhibitor that induces cell-cycle arrest and apoptosis. It is a cell-permeable thiazolidinone that specifically inhibits the c-Myc-Max interaction and prevents transactivation of c-Myc target gene expression. 10058-F4 inhibits tumor cell growth in a c-Myc-dependent manner both in vitro and in vivo. BI-6C9 is a tBid inhibitor and antiapoptotic. GNF-2 belongs to a new class of Bcr-ab1 inhibitors. GNF-2 appears to bind to the myristoyl binding pocket, an allosteric site distant from the active site, stabilizing the inactive form of the kinase. It inhibits Bcr-ab1 phosphorylation with an IC₅₀ of 267 nM, but does not inhibit a panel of 63 other kinases, including native c-Ab1, and shows complete lack of toxicity towards cells not expressing Bcr-Ab1. GNF-2 shows great potential for a new class of inhibitor to study Bcr-ab1 activity and to treat resistant Chronic myelogenous leukemia (CML), which is caused the Bcr-Ab1 oncoprotein. Pifithrin-α is a reversible inhibitor of p53-mediated apoptosis and p53-dependent gene transcription such as cyclin G, p21/waf1, and mdm2 expression. Pifithrin-α enhances cell survival after genotoxic stress such as UV irradiation and treatment with cytotoxic compounds including doxorubicin, etopoxide, paclitaxel, and cytosine-13-D-arabinofuranoside. Pifithrin-α protects mice from lethal whole body γ-irradiation without an increase in cancer incidence.

### Radiation Therapy

Radiation therapy (or radiotherapy) is the medical use of ionizing radiation as part of cancer treatment to control malignant cells. Radiotherapy may be used for curative or adjuvant cancer treatment. It is used as palliative treatment (where cure is not possible and the aim is for local disease control or symptomatic relief) or as therapeutic treatment (where the therapy has survival benefit and it can be curative). Radiotherapy is used for the treatment of malignant tumors and may be used as the primary therapy. It is also common to combine radiotherapy with surgery, chemotherapy, hormone therapy or some mixture of the three. Most common cancer types can be treated with radiotherapy in some way. The precise treatment intent (curative, adjuvant, neoadjuvant, therapeutic, or palliative) will depend on the tumour type, location, and stage, as well as the general health of the patient.

Radiation therapy is commonly applied to the cancerous tumor. The radiation fields may also include the draining of lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion.

Radiation therapy works by damaging the DNA of cells. The damage is caused by a photon, electron, proton, neutron, or ion beam directly or indirectly ionizing the atoms which make up the DNA chain. Indirect ionization happens as a result of the ionization of water, forming free radicals, notably hydroxyl radicals, which then damage the DNA. In the most common forms of radiation therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly. Proton radiotherapy works by sending protons with varying kinetic energy to precisely stop at the tumor.

Gamma rays are also used to treat some types of cancer including breast cancer. In the procedure called gamma-knife surgery, multiple concentrated beams of gamma rays are directed on the growth in order to kill the cancerous cells. The beams are aimed from different angles to focus the radiation on the growth while minimizing damage to the surrounding tissues.

### Gene Therapy Agents

Gene therapy agents insert copies of genes into a specific set of a patient's cells, and can target both cancer and non-cancer cells. The goal of gene therapy can be to replace altered genes with functional genes, to stimulate a patient's immune response to cancer, to make cancer cells more sensitive to chemotherapy, to place “suicide” genes into cancer cells, or to inhibit angiogenesis. Genes may be delivered to target cells using viruses, liposomes, or other carriers or vectors. This may be done by injecting the gene-carrier composition into the patient directly, or ex vivo, with infected cells being introduced back into a patient. Such compositions are suitable for use in the present invention.

### Adjuvant Therapy

Adjuvant therapy is a treatment given after the primary treatment to increase the chances of a cure. Adjuvant therapy may include chemotherapy, radiation therapy, hormone therapy, or biological therapy.

Because the principal purpose of adjuvant therapy is to kill any cancer cells that may have spread, treatment is usually systemic (uses substances that travel through the bloodstream, reaching and affecting cancer cells all over the body). Adjuvant therapy for breast cancer involves chemotherapy or hormone therapy, either alone or in combination.

Adjuvant chemotherapy is the use of drugs to kill cancer cells. Research has shown that using chemotherapy as adjuvant therapy for early stage breast cancer helps prevent the original cancer from returning. Adjuvant chemotherapy is usually a combination of anticancer drugs, which has been shown to be more effective than a single anticancer drug.

Adjuvant hormone therapy deprives cancer cells of the female hormone estrogen, which some breast cancer cells need to grow. Most often, adjuvant hormone therapy is treatment with the drug tamoxifen. Research has shown that when tamoxifen is used as adjuvant therapy for early stage breast cancer, it helps prevent the original cancer from returning and also helps prevent the development of new cancers in the other breast.

The ovaries are the main source of estrogen prior to menopause. For premenopausal women with breast cancer, adjuvant hormone therapy may involve tamoxifen to deprive the cancer cells of estrogen. Drugs to suppress the production of estrogen by the ovaries are under investigation. Alternatively, surgery may be performed to remove the ovaries.

Radiation therapy is sometimes used as a local adjuvant treatment. Radiation therapy is considered adjuvant treatment when it is given before or after a mastectomy. Such treatment is intended to destroy breast cancer cells that have spread to nearby parts of the body, such as the chest wall or lymph nodes. Radiation therapy is part of primary therapy, not adjuvant therapy, when it follows breast-sparing surgery.

### Neoadjuvant Therapy

Neoadjuvant therapy refers to a treatment given before the primary treatment. Examples of neoadjuvant therapy include chemotherapy, radiation therapy, and hormone therapy. In treating breast cancer, neoadjuvant therapy allows patients with large breast cancer to undergo breast-conserving surgery.

### Oncolytic Viral Therapy

Viral therapy for cancer utilizes a type of viruses called oncolytic viruses. An oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site.

There are two main approaches for generating tumor selectivity: transductional and non-transductional targeting. Transductional targeting involves modifying the specificity of viral coat protein, thus increasing entry into target cells while reducing entry to non-target cells. Non-transductional targeting involves altering the genome of the virus so it can only replicate in cancer cells. This can be done by either transcription targeting, where genes essential for viral replication are placed under the control of a tumor-specific promoter, or by attenuation, which involves introducing deletions into the viral genome that eliminate functions that are dispensable in cancer cells, but not in normal cells. There are also other, slightly more obscure methods.

Chen et al (2001) used CV706, a prostate-specific adenovirus, in conjunction with radiotherapy on prostate cancer in mice. The combined treatment results in a synergistic increase in cell death, as well as a significant increase in viral burst size (the number of virus particles released from each cell lysis).

ONYX-015 has undergone trials in conjunction with chemotherapy. The combined treatment gives a greater response than either treatment alone, but the results have not been entirely conclusive. ONYX-015 has shown promise in conjunction with radiotherapy.

Viral agents administered intravenously can be particularly effective against metastatic cancers, which are especially difficult to treat conventionally. However, bloodborne viruses can be deactivated by antibodies and cleared from the blood stream quickly e.g., by Kupffer cells (extremely active phagocytic cells in the liver, which are responsible for adenovirus clearance). Avoidance of the immune system until the tumour is destroyed could be the biggest obstacle to the success of oncolytic virus therapy. To date, no technique used to evade the immune system is entirely satisfactory. It is in conjunction with conventional cancer therapies that oncolytic viruses show the most promise, since combined therapies operate synergistically with no apparent negative effects.

The specificity and flexibility of oncolytic viruses means they have the potential to treat a wide range of cancers including breast cancer with minimal side effects. Oncolytic viruses have the potential to solve the problem of selectively killing cancer cells.

### Nanotherapy

Nanometer-sized particles have novel optical, electronic, and structural properties that are not available from either individual molecules or bulk solids. When linked with tumor-targeting moieties, such as tumor-specific ligands or monoclonal antibodies, these nanoparticles can be used to target cancer-specific receptors, tumor antigens (biomarkers), and tumor vasculatures with high affinity and precision. The formulation and manufacturing process for cancer nanotherapy is disclosed in U.S. Pat. No. 7,179,484, and article M. N. Khalid, P. Simard, D. Hoarau, A. Dragomir, J. Leroux, Long Circulating Poly(Ethylene Glycol) Decorated Lipid Nanocapsules Deliver Docetaxel to Solid Tumors, Pharmaceutical Research, 23(4), 2006, all of which are herein incorporated by reference in their entireties.

### RNA Therapy

RNA including but not limited to siRNA, shRNA, or microRNA may be used to modulate gene expression and treat cancers. Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises a nucleotide sequence that is homologous to the target nucleic acid sequence.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

Certain RNA inhibiting agents may be utilized to inhibit the expression or translation of messenger RNA (“mRNA”) that is associated with a cancer phenotype. Examples of such agents suitable for use herein include, but are not limited to, short interfering RNA (“siRNA”), ribozymes, and antisense oligonucleotides. Specific examples of RNA inhibiting agents suitable for use herein include, but are not limited to, Cand5, Sirna-027, fomivirsen, and angiozyme.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives.

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Chemopreventative Agents

Certain pharmaceutical agents can be used to prevent initial occurrences of cancer, or to prevent recurrence or metastasis. Such chemopreventative agents in combination with a method provided herein may be used to treat and prevent the recurrence of cancer. Examples of chemopreventative agents suitable for use herein include, but are not limited to, tamoxifen, raloxifene, tibolone, bisphosphonate, ibandronate, estrogen receptor modulators, aromatase inhibitors (letrozole, anastrozole), luteinizing hormone-releasing hormone agonists, goserelin, vitamin A, retinal, retinoic acid, fenretinide, 9-cis-retinoid acid, 13-cis-retinoid acid, all-trans-retinoic acid, isotretinoin, tretinoid, vitamin B6, vitamin B12, vitamin C, vitamin D, vitamin E, cyclooxygenase inhibitors, non-steroidal anti-inflammatory drugs (NSAIDs), aspirin, ibuprofen, celecoxib, polyphenols, polyphenol E, green tea extract, folic acid, glucaric acid, interferon-alpha, anethole dithiolethione, zinc, pyridoxine, finasteride, doxazosin, selenium, indole-3-carbinal, alpha-difluoromethylornithine, carotenoids, beta-carotene, lycopene, antioxidants, coenzyme Q10, flavonoids, quercetin, curcumin, catechins, epigallocatechin gallate, N-acetylcysteine, indole-3-carbinol, inositol hexaphosphate, isoflavones, glucanic acid, rosemary, soy, saw palmetto, and calcium. An additional example of chemopreventative agents suitable for use in the present invention is cancer vaccines. These can be created through immunizing a patient with all or part of a cancer cell type that is targeted by the vaccination process.

### Methods of Treating Breast Cancer (e.g., Locally Advanced Breast Cancer or Metastatic Breast Cancer)

Provided herein are methods of treating breast cancer (e.g., locally advanced or metastatic breast cancer) in a patient comprising administration of a) 4-iodo-3-nitrobenzamide, or a metabolite thereof, or a pharmaceutically acceptable salt or solvate thereof, and b) irinotecan, or a pharmaceutically acceptable salt or solvate thereof, to said patient. In some embodiments, the breast cancer is locally advanced breast cancer. In some embodiments, the breast cancer is progressing locally advanced breast cancer. In some embodiments, the breast cancer is metastatic breast cancer. In some embodiments, 4-iodo-3-nitrobenzamide or a pharmaceutically acceptable salt thereof (e.g., 4-iodo-3-nitrobenzamide) is administered to the patient. In some embodiments, irinotecan is administered to the patient. In some embodiments, the 4-iodo-3-nitrobenzamide or a pharmaceutically acceptable salt thereof (e.g., 4-iodo-3-nitrobenzamide) is administered at abut 4 mg/kg to about 25 mg/kg (e.g., about 5.6 mg/kg twice weekly, about 8 mg/kg twice weekly, or 11.2 mg/kg once weekly). In some embodiments, the irinotecan is administered at about 80 mg/m² to about 125 mg/m² once weekly.

In some embodiments, the breast cancer (e.g., locally advanced breast cancer or metastatic breast cancer) is hormone receptor-negative (“HR-negative”) breast cancer. In some embodiments, the breast cancer (e.g., locally advanced breast cancer or metastatic breast cancer) is negative for at least one of: estrogen receptor (“ER”), progesterone receptor (“PR”) or human epidermal growth factor receptor 2 (“HER2”). In some embodiments, the breast cancer (e.g., locally advanced breast cancer or metastatic breast cancer) is negative for at least one of: ER, PR or HER2; and the breast cancer (e.g., locally advanced breast cancer or metastatic breast cancer) is positive for at least one of ER, PR or HER2. In some embodiments, the breast cancer (e.g., locally advanced breast cancer or metastatic breast cancer) is HR-negative breast cancer. In some embodiments, the breast cancer (e.g., locally advanced breast cancer or metastatic breast cancer) is an ER-negative breast cancer. In some embodiments, the breast cancer (e.g., locally advanced breast cancer or metastatic breast cancer) is ER-negative and HER2-positive. In some embodiments, the breast cancer (e.g., locally advanced breast cancer or metastatic breast cancer) is ER-negative and PR-positive. In some embodiments, the breast cancer (e.g., locally advanced breast cancer or metastatic breast cancer) is ER-negative and both HER2-positive and PR-positive. In some embodiments, the breast cancer (e.g., locally advanced breast cancer or metastatic breast cancer) is a PR-negative breast cancer. In some embodiments, the breast cancer (e.g., locally advanced breast cancer or metastatic breast cancer) is PR-negative and ER-positive. In some embodiments, the breast cancer (e.g., locally advanced breast cancer or metastatic breast cancer) is PR-negative and HER2-positive. In some embodiments, the breast cancer (e.g., locally advanced breast cancer or metastatic breast cancer) is PR-negative and both ER-positive and HER2-positive. In some embodiments, the breast cancer (e.g., locally advanced breast cancer or metastatic breast cancer) is a HER2-negative breast cancer. In some embodiments, the breast cancer (e.g., locally advanced breast cancer or metastatic breast cancer) is HER2-negative and ER-positive. In some embodiments, the breast cancer (e.g., locally advanced breast cancer or metastatic breast cancer) is HER2-negative and PR-positive. In some embodiments, the breast cancer (e.g., locally advanced breast cancer or metastatic breast cancer) is HER2-negative and both ER-positive and PR-positive. In some embodiments, the breast cancer (e.g., locally advanced breast cancer or metastatic breast cancer) is ER-negative and PR-negative. In some embodiments, the breast cancer (e.g., locally advanced breast cancer or metastatic breast cancer) is ER-negative, PR-negative and HER-2 positive. In some embodiments, the breast cancer (e.g., locally advanced breast cancer or metastatic breast cancer) is ER-negative and HER2-negative. In some embodiments, the breast cancer (e.g., locally advanced breast cancer or metastatic breast cancer) is ER-negative, HER2-negative and PR-positive. In some embodiments, the breast cancer (e.g., locally advanced breast cancer or metastatic breast cancer) is PR-negative and HER2-negative. In some embodiments, the breast cancer (e.g., locally advanced breast cancer or metastatic breast cancer) is PR-negative, HER2-negative and ER-positive. In some embodiments, the breast cancer (e.g., locally advanced breast cancer or metastatic breast cancer) is ER-negative, PR-negative and HER2-negative.

In some embodiments, the breast cancer (e.g., locally advanced breast cancer or metastatic breast cancer) does not overexpress HER2. In some embodiments, the breast cancer (e.g., locally advanced breast cancer or metastatic breast cancer) overexpresses HER2. In some embodiments, the breast cancer (e.g., locally advanced breast cancer or metastatic breast cancer) is negative for ER and/or negative for PR. In some embodiments, the breast cancer (e.g., locally advanced breast cancer or metastatic breast cancer) is positive for ER and/or positive for PR. In some embodiments, the breast cancer (e.g., locally advanced breast cancer or metastatic breast cancer) is negative for estrogen receptor (ER) expression, negative for progesterone receptor (PR) expression, and does not overexpress HER2. In some embodiments, the breast cancer (e.g., locally advanced breast cancer or metastatic breast cancer) expresses estrogen receptor (ER), progesterone receptor (PR), and/or overexpresses HER2.

In some embodiments, the breast cancer (e.g., locally advanced breast cancer or metastatic breast cancer) is deficient in homologous recombination DNA repair. In some embodiments, the breast cancer (e.g., locally advanced breast cancer or metastatic breast cancer) is BRCA-deficient. In some embodiments, the breast cancer (e.g., locally advanced breast cancer or metastatic breast cancer) is BRCA1-deficient. In some embodiments, the breast cancer (e.g., locally advanced breast cancer or metastatic breast cancer) is BRCA2-deficient. In some embodiments, the breast cancer (e.g., locally advanced breast cancer or metastatic breast cancer) is BRCA1-deficient and BRCA2-deficient. In some embodiments, the patient has breast cancer tissue expressing varying level of BRCA (e.g., BRCA1) compared to normal breast tissue, for example, the patient has reduced level of BRCA (e.g., BRCA1) expression compared to normal breast tissue. In some embodiments, the BRCA (e.g., BRCA1) expression is reduced by at least about 1.5 fold (e.g., at least about any of 2 fold, 3 fold, 5 fold, 6 fold, 7 fold, 8 fold, 9 fold, or 10 fold). In some embodiments, the breast cancer comprises at least one mutation in BRCA1 and/or at least one mutation in BRCA2.

In some embodiments, the breast cancer is metastatic breast cancer. In some embodiments, the breast cancer is locoregional. In some embodiments, the breast cancer is progressing locoregional. In some embodiments, the metastasis is distant metastasis. In some embodiments, the metastasis is systemic metastasis. In some embodiments, the metastasis is brain metastasis. In some embodiments, the breast cancer is locally advanced breast cancer. In some embodiments, the breast cancer is progressing locally advanced breast cancer.

In some embodiments, the breast cancer (e.g., locally advanced or metastatic breast cancer) has been previously treated. Prior treatments include, but are not limited to, chemotherapy, radiation, hormonal therapy and/or surgery. For example, prior treatment may include an anthracycline (e.g., daunorubicin (daunomycin), daunorubicin (liposomal), doxorubicin (adriamycin), doxorubicin (liposomal), epirubicin, idarubicin, valrubicin, or mitoxantrone), an anthraquinone (e.g., 9,10-anthraquinone or 9,10-dioxoanthracene, 1,2-, 1,4-, or 2,6-anthraquinone), and/or a taxane (e.g., paclitaxel, docetaxel). For example, in some embodiments, the patient being treated using any one of the methods provided herein has received prior chemotherapy treatment comprising at least one regimen selected from the group consisting of an anthracycline (e.g., daunorubicin (daunomycin), daunorubicin (liposomal), doxorubicin (adriamycin), doxorubicin (liposomal), epirubicin, idarubicin, valrubicin, or mitoxantrone), an anthraquinone (e.g., 9,10-anthraquinone or 9,10-dioxoanthracene, 1,2-, 1,4-, or 2,6-anthraquinone) and a taxane (e.g., paclitaxel, docetaxel). In some embodiments, the breast cancer (e.g., metastatic breast cancer) is refractory to standard treatment or for which no standard therapy is available. In some embodiments, the breast cancer is advanced breast cancer. In some embodiments, the advanced breast cancer is refractory to standard treatment or for which no standard therapy is available. In some embodiments, the patient is refractory to at least one regimen selected from the group consisting of an anthracycline (e.g., daunorubicin (daunomycin), daunorubicin (liposomal), doxorubicin (adriamycin), doxorubicin (liposomal), epirubicin, idarubicin, valrubicin, or mitoxantrone), an anthraquinone (e.g., 9,10-anthraquinone or 9,10-dioxoanthracene, 1,2-, 1,4-, or 2,6-anthraquinone) and a taxane (e.g., paclitaxel, docetaxel). In some embodiments, the patient has received maximum of one adjuvant regimen and two regimens for metastatic disease (whether or not these are based on an anthracycline or a taxane) prior to a treatment described herein. In some embodiments, the breast cancer is metastatic breast cancer. In some embodiments, the breast cancer is locally advanced breast cancer. In some embodiments, the patient has a lesion of at least 2.0 centimeter (e.g., a lesion of bi-dimensionally measuring at least 2.0 centimeter that is, for example, assessed by computed tomography, magnetic resonance imaging, or ultra-sonography).

Provided herein are methods of treating metastatic triple negative breast cancer (ER−, PR−, HER2−) in a patient, comprising administering to the patient having metastatic triple negative breast cancer an effective amount of 4-iodo-3-nitrobenzamide, a metabolite thereof, or a pharmaceutically acceptable salt thereof, and irinotecan. In some embodiments, the metastasis comprises brain metastases. Also provided herein are methods of treating metastatic triple negative breast cancer in a patient, where the triple negative breast cancer has metastasized to the brain, comprising administering to the patient having metastatic triple negative breast cancer an effective amount of 4-iodo-3-nitrobenzamide and irinotecan.

Also provided herein are methods of treating a patient with breast cancer brain metastasis comprising administering to the patient an effective amount of (a) 4-iodo-3-nitrobenzamide or a metabolite thereof or a pharmaceutically acceptable salt thereof (e.g., 4-iodo-3-nitrobenzamide) and (b) irinotecan or a pharmaceutically acceptable salt thereof (e.g., irinotecan), wherein the breast cancer is ER-negative, PR-negative, and HER2-nonoverexpressing. In some embodiments, the effective amount is administered over a 21-day treatment cycle, wherein 4-iodo-3-nitrobenzamide or a metabolite thereof or a pharmaceutically acceptable salt thereof is administered to the patient at about 5.6 mg/kg on days 1, 4, 8, 11 of the treatment cycle, and wherein irinotecan or a pharmaceutically acceptable salt thereof is administered at about 125 mg/m² on days 1 and 8 of the cycle. In some embodiments, the effective amount is administered over a 21-day treatment cycle, wherein 4-iodo-3-nitrobenzamide or a metabolite thereof or a pharmaceutically acceptable salt thereof is administered to the patient at about 11.2 mg/kg on days 1 and 8 of the treatment cycle, and wherein irinotecan or a pharmaceutically acceptable salt thereof is administered at about 125 mg/m² on days 1 and 8 of the cycle. In some embodiments, the effective amount produces at least one therapeutic effect selected from the group consisting of reduction in size of a breast tumor, reduction in metastasis, complete remission, partial remission, stable disease, increase in overall response rate, or a pathologic complete response. In some embodiments, the brain metastasis is at least about 0.5 centimeter.

Also provided herein are methods of treating metastatic breast cancer brain metastasis in a patient comprising administering to the patient having the metastatic breast cancer brain metastasis an effective amount of (a) 4-iodo-3-nitrobenzamide or a metabolite thereof or a pharmaceutically acceptable salt thereof and (b) irinotecan. In some embodiments, the breast cancer is ER-negative, PR-negative, and HER2-nonoverexpressing. In some embodiments, the brain metastasis is at least about or larger than about 0.5 centimeter (e.g., brain metastasis measuring at least about or larger than about 0.5 centimeter in longest dimension). In some embodiments, the brain metastasis (e.g., brain metastasis measuring at least about or larger than about 0.5 centimeter) is new and/or progressive brain metastasis following radiation therapy (e.g., central nervous system (“CNS”) radiation therapy or intracranial radiation therapy). In some embodiments, the brain metastasis (e.g., brain metastasis measuring at least about or larger than about 0.5 centimeter) is new and/or progressive brain metastasis following radiation therapy (e.g., central nervous system (“CNS”) radiation therapy or intracranial radiation therapy) for breast cancer brain metastases. In some embodiments, the brain metastasis (e.g., brain metastasis measuring at least about or larger than about 0.5 centimeter) is new and/or progressive brain metastasis after prior radiation therapy (e.g., after prior central nervous system radiation therapy or after prior intracranial radiation therapy). In some embodiments, the brain metastasis (e.g., brain metastasis measuring at least about or larger than about 0.5 centimeter) is new and/or progressive brain metastasis after prior radiation therapy (e.g., after prior central nervous system radiation therapy or after prior intracranial radiation therapy) for breast cancer brain metastases. In some embodiments, the brain metastasis is new brain metastasis (e.g., new brain metastasis measuring at least about or larger than about 0.5 centimeter) after the radiation therapy. In some embodiments, the brain metastasis is progressive brain metastasis (e.g., progressive brain metastasis measuring at least about or larger than about 0.5 centimeter) after the radiation therapy. In some embodiments, the brain metastasis (e.g., brain metastasis measuring at least about or larger than about 0.5 centimeter) is asymptomatic and the patient is CNS-radiation therapy naïve patient. In some embodiments, the patient has no prior radiation therapy (e.g., prior intracranial radiation therapy). In some embodiments, the brain metastasis (e.g., brain metastasis measuring at least about or larger than about 0.5 centimeter) is new brain metastasis and the patient has no prior intracranial radiation therapy and/or the intracranial radiation therapy is not emergently indicated for the patient. In some embodiments, the brain metastasis (e.g., brain metastasis measuring at least about or larger than about 0.5 centimeter) is new brain metastasis (e.g., brain metastasis is found within 2 weeks of initiation of a therapy such as a protocol-based therapy) and the patient is intracranial radiation-naïve patient for whom intracranial radiation therapy is not emergently indicated. In some embodiments, the patient does not have leptomeningeal disease (e.g., the patient does not have diffuse leptomeningeal disease). The effective amount of 4-iodo-3-nitrobenzamide or a metabolite thereof or a pharmaceutically acceptable salt thereof and/or irinotecan may be according to any of the dosages described herein. In some embodiments, 4-iodo-3-nitrobenzamide or a metabolite thereof or a pharmaceutically acceptable salt thereof is administered at about 4 mg/kg to about 25 mg/kg (e.g., about 5.6 mg/kg or about 11.2 mg/kg). In some embodiments, irinotecan is administered at about 50 mg/m² to about 200 mg/m² (e.g., about 125 mg/m²). In some embodiments, the treatment comprises at least one treatment cycle of 21-days, wherein 4-iodo-3-nitrobenzamide or a metabolite thereof or a pharmaceutically acceptable salt thereof is administered at about 5.6 mg/kg on days 1, 4, 8, and 11 of the cycle or about 11.2 mg/kg on days 1 and 8 of the treatment cycle, and/or wherein irinotecan is administered at about 125 mg/m² on days 1 and 8 of the cycle. In some embodiments, 4-iodo-3-nitrobenzamide or a metabolite thereof or a pharmaceutically acceptable salt thereof is administered intravenously. In some embodiments, irinotecan is administered intravenously.

In some embodiments, the breast cancer is carcinoma in situ. In some embodiments, the breast cancer is infiltrating (or invasive) carcinoma. In some embodiments, the breast cancer is lobular carcinoma or ductal carcinoma. In some embodiments, the breast cancer is lobular carcinoma in situ or a ductal carcinoma in situ. In some embodiments, the breast cancer is infiltrating (or invasive) lobular carcinoma or infiltrating (or invasive) ductal carcinoma. In some embodiments, the breast cancer is mammary ductal carcinoma. In some embodiments, the breast cancer is intraductal, invasive, comedo, inflammatory, medullary with lymphocytic infiltrate, mucinous (colloid), papillary, scirrhous, or tubular ductal carcinoma. Other cancers of the breast that can be treated by the methods provided herein are medullary carcinomas, colloid carcinomas, tubular carcinomas, inflammatory breast cancer, nipple carcinoma, and paget disease with intraductal carcinoma or with invasive ductal carcinoma. In some embodiments, the breast cancer described herein is metastatic breast cancer. In some embodiments, the breast cancer described herein is locally advanced breast cancer. In some embodiments, the breast cancer (e.g., locally advanced breast cancer or metastatic breast cancer) is the Luminal B subtype, Luminal A subtype, normal-like subtype, basal-like subtype, claudin-low subtype, or HER2-enriched subtype. In some embodiments, the breast cancer described herein is metastatic breast cancer. In some embodiments, the metastasis comprises brain metastases. In some embodiments, the breast cancer described herein is locally advanced breast cancer.

In some embodiments, the breast cancer is any of stage 0, stage I, stage II, stage III, or stage IV breast cancer. In some embodiments, the breast cancer is inflammatory breast cancer. In some embodiments, the breast cancer is stage II and/or stage III. In some embodiments, the breast cancer is stage II. In some embodiments, the breast cancer is stage IIIA breast cancer. In some embodiments, the breast cancer is early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some specific embodiments, the breast cancer is in a neoadjuvant setting. In some embodiments, there are provided methods of treating cancer at advanced stage(s). In some embodiments, the patient does not have bilateral breast cancer. In some embodiments, the patient does not have multicentric breast cancer.

In some embodiments, a method provided herein is used to treat a primary breast tumor. In some embodiments, a method provided herein is used to treat a metastatic breast cancer (that is, cancer that has metastasized from the primary tumor). In some embodiments, the breast cancer is early stage cancer, non-metastatic cancer, primary cancer, advanced cancer, locally advanced cancer, metastatic cancer, cancer in remission, or recurrent cancer. In some embodiments, the breast cancer has reoccurred after remission. In some embodiments, the breast cancer is progressive cancer. In some embodiments, the breast cancer is localized resectable, localized unresectable, or unresectable. In some embodiments, the breast cancer is locoregional. In some embodiments, the breast cancer is progressing locoregional. In some embodiments, the metastasis is distant metastasis. In some embodiments, the metastasis is systemic metastasis. In some embodiments, the metastasis comprises brain metastasis. In some embodiments, the metastasis is brain metastasis. In some embodiments, the breast cancer is substantially refractory to hormone therapy. In some embodiments, the patient has breast adenocarcinoma (e.g., the breast cancer is breast adenocarcinoma).

In some embodiments, a method provided herein is used in an adjuvant setting. In some embodiments, a method provided herein is used in a neoadjuvant setting, i.e., the method may be carried out before the primary/definitive therapy such as surgery (e.g., surgery for removing breast cancer tissue from a patient). For example, a method provided herein may be practiced before a surgery for removing breast cancer tissue from the patient. In some embodiments, a method provided herein may be used to treat a patient who has previously been treated. In some embodiments, a method provided herein is used to treat a patient who has not previously been treated. For example, the patient having breast cancer has not received chemotherapy, hormone therapy, surgery, and/or radiation prior to receiving a treatment provided herein. In some embodiments, a method provided herein is used to treat an individual at risk for developing cancer, but has not been diagnosed with cancer. In some embodiments, a method provided herein is used as a first line therapy. In some embodiments, a method provided herein is used as a second line therapy.

In some embodiments, the patient has not received a prior chemotherapy comprising 4-iodo-3-nitrobenzamide or a metabolite or a pharmaceutically acceptable salt thereof. In some embodiments, the patient has not received a prior chemotherapy comprising a PARP inhibitor (e.g., Olaparib, ABT-888 (Veliparib), AG014699, CEP 9722, MK 4827, KU-0059436 (AZD2281), or LT-673). In some embodiments, the patient has not received a prior chemotherapy comprising gemcitabine. In some embodiments, the patient has not received a prior chemotherapy comprising carboplatin. In some embodiments, the patient has not received a prior chemotherapy comprising cisplatin.

In some embodiments, a method provided herein is used to treat an individual (e.g., human) who has been diagnosed with or is suspected of having breast cancer. In some embodiments, the individual may be a human who exhibits one or more symptoms associated with breast cancer. In some embodiments, the individual may have advanced disease or a lesser extent of disease, such as low tumor burden. In some embodiments, the individual is at an early stage of a breast cancer. In some embodiments, the individual is at an advanced stage of breast cancer. In some of the embodiments, the individual may be a human who is genetically or otherwise predisposed (e.g., risk factor) to developing breast cancer who has or has not been diagnosed with breast cancer. In some embodiments, these risk factors include, but are not limited to, age, sex, race, diet, history of previous disease, presence of precursor disease, genetic (e.g., hereditary) considerations, and environmental exposure (e.g., cigarette, pipe, or cigar smoking, exposure to second-hand smoke, radon, arsenic, asbestos, chromates, chloromethyl ethers, nickel, polycyclic aromatic hydrocarbons, radon progeny, other agents, or air pollution).

In some embodiments of any of the methods described herein, an individual (e.g., human) who has been diagnosed with or is suspected of having breast cancer can be treated. In some embodiments, the individual is human. In some embodiments, the individual is at least about any of 35, 40, 45, 50, 55, 60, 65, 70, 75, 80, or 85 years old. In some embodiments, the individual is male. In some embodiments, the individual is a female. In some embodiments, the individual has any of the types of breast cancer described herein. In some embodiments, the individual has a single lesion at presentation. In some embodiments, the individual has multiple lesions at presentation.

In some embodiments, the effective amount produces at least one therapeutic effect selected from the group consisting of reduction in size of a breast tumor, reduction in metastasis, complete remission, partial remission, stable disease, increase in overall response rate, or a pathologic complete response. In some embodiments, the treatment produces complete response, partial response, or stable disease. The clinical efficacy parameters described herein may be measured according to RECIST version 1.1 criteria, which is described in Eisenhauer E A et al. 2009, Eur J. Cancer., 45(2):228-47, the disclosure of which is incorporated by reference in its entirety.

In some embodiments, there is provided a method of reducing breast tumor size in a patient comprising administering to the patient 4-iodo-3-nitrobenzamide or a metabolite or pharmaceutically acceptable salt thereof, in combination with irinotecan. In some embodiments, the patient has locally advanced breast cancer. In some embodiments, the patient has metastatic breast cancer. The breast cancer may be any of the breast cancers described herein. The dosing regimen may be any of the dosing regimens described herein.

In some embodiments, there is provided a method of reducing breast cancer metastasis (e.g., breast cancer brain metastasis) in a patient comprising administering to the patient 4-iodo-3-nitrobenzamide or a metabolite or pharmaceutically acceptable salt thereof, in combination with irinotecan. The breast cancer may be any of the breast cancers described herein. The dosing regimen may be any of the dosing regimens described herein.

In some embodiments, a treatment described herein reduces breast tumor size by about or at least about any of 10%, 20%, 30%, 40%, 50%, 60%, 70%, 80% or 90%. In some embodiments, a treatment described herein reduces breast cancer metastasis (e.g., brain metastasis) by about or at least about any of 10%, 20%, 30%, 40%, 50%, 60%, 70%, 80% or 90%.

In one aspect, provided herein are methods of treating breast cancer (e.g., metastatic breast cancer) in a patient, comprising administering to the patient having breast cancer (e.g., metastatic breast cancer) an effective amount of 4-iodo-3-nitrobenzamide or a metabolite or pharmaceutically acceptable salt thereof, in combination with irinotecan. In some embodiments, at least one therapeutic effect is obtained, the at least one therapeutic effect being reduction in size of a breast tumor, reduction in metastasis, complete remission, partial remission, pathologic complete response, increase in overall response rate or stable disease. In some embodiments, clinical efficacy of the combination of a) 4-iodo-3-nitrobenzamide or a metabolite thereof or a pharmaceutically acceptable salt thereof, and b) irinotecan may be determined by measuring the clinical benefit rate (CBR). In some emobidiments, the clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD≧6 months. The CBR for combination therapy with a) 4-iodo-3-nitrobenzamide or a metabolite thereof or a pharmaceutically acceptable salt or solvate thereof, and b) irinotecan may be compared to that of therapy with irinotecan when administered without 4-iodo-3-nitrobenzamide. In some embodiments, the improvement of clinical benefit rate is greater than about any of 20%, 30%, 40%, 50%, 60%, 70%, 80% or 90%.

### Staging of Breast Cancer

Stage 0 may be used to describe non-invasive breast cancers, such as DCIS and LCIS. In stage 0, there is no evidence of cancer cells or non-cancerous abnormal cells breaking out of the part of the breast in which they started, or of getting through to or invading neighboring normal tissue.

Stage I may describe invasive breast cancer (cancer cells are breaking through to or invading neighboring normal tissue) in which the tumor measures up to 2 centimeters, and no lymph nodes are involved.

Stage II may be divided into subcategories known as IIA and IIB. Stage IIA may describe invasive breast cancer in which no tumor can be found in the breast, but cancer cells are found in the axillary lymph nodes (the lymph nodes under the arm), or the tumor measures 2 centimeters or less and has spread to the axillary lymph nodes, or the tumor is larger than 2 centimeters but not larger than 5 centimeters and has not spread to the axillary lymph nodes. Stage IIB may describe invasive breast cancer in which: the tumor is larger than 2 but no larger than 5 centimeters and has spread to the axillary lymph nodes, or the tumor is larger than 5 centimeters but has not spread to the axillary lymph nodes.

Stage III may be divided into subcategories known as IIIA, IIIB, and IIIC. Stage 111A may describe invasive breast cancer in which either (1) no tumor is found in the breast; cancer is found in axillary lymph nodes that are clumped together or sticking to other structures, or cancer may have spread to lymph nodes near the breastbone, or (2) the tumor is 5 centimeters or smaller and has spread to axillary lymph nodes that are clumped together or sticking to other structures, or (3) the tumor is larger than 5 centimeters and has spread to axillary lymph nodes that are clumped together or sticking to other structures. Stage IIIB may describe invasive breast cancer in which (1) the tumor may be any size and has spread to the chest wall and/or skin of the breast and (2) may have spread to axillary lymph nodes that are clumped together or sticking to other structures, or cancer may have spread to lymph nodes near the breastbone. Inflammatory breast cancer may be considered at least stage IIIB Stage IIIC may describe invasive breast cancer in which (1) there may be no sign of cancer in the breast or, if there is a tumor, it may be any size and may have spread to the chest wall and/or the skin of the breast, and (2) the cancer has spread to lymph nodes above or below the collarbone, and (3) the cancer may have spread to axillary lymph nodes or to lymph nodes near the breastbone.

Stage IV may describe invasive breast cancer that has spread beyond the breast and nearby lymph nodes to other organs of the body, such as the lungs, distant lymph nodes, skin, bones, liver, or brain.

### Dosing Regimen, Routes of Administration, and Formulations

In the methods of treating breast cancer provided herein, any one of the dosage or dosing schedule described herein may be used.

The dosage of 4-iodo-3-nitrobenzamide or a metabolite thereof or a pharmaceutically acceptable salt thereof may vary depending upon the patient's age, height, weight, overall health, etc. In some embodiments, the dosage of 4-iodo-3-nitrobenzamide or a metabolite thereof or a pharmaceutically acceptable salt thereof is in the range of any one of about 0.1 mg/kg to about 50 mg/kg, about 1 mg/kg to about 50 mg/kg, about 1 mg/kg to about 100 mg/kg, about 1 mg/kg to about 25 mg/kg, about 1 mg/kg to about 15 mg/kg, about 1 mg/kg to about 6 mg/kg, about 2 to about 70 mg/kg, about 2 mg/kg to about 50 mg/kg, about 2 mg/kg to about 40 mg/kg, about 3 mg/kg to about 30 mg/kg, about 4 mg/kg to about 20 mg/kg, about 4 to about 15 mg/kg, about 4 to about 100 mg, about 4 to about 25 mg/kg, about 5 to about 15 mg/kg, about 5 to about 10 mg/kg, about 50 to about 100 mg/kg or about 25 to about 75 mg/kg. In some embodiments, the dosage of 4-iodo-3-nitrobenzamide or a metabolite thereof or a pharmaceutically acceptable salt thereof is greater than or at least about any of 1 mg/kg, 2 mg/kg, 3 mg/kg, 4 mg/kg, 5 mg/kg, 6 mg/kg, 7 mg/kg, 8 mg/kg, 8.5 mg/kg, 9 mg/kg, 9.5 mg/kg, 10 mg/kg, 12 mg/kg, or 15 mg/kg. In some embodiments, the dosage of 4-iodo-3-nitrobenzamide or a metabolite thereof or a pharmaceutically acceptable salt thereof is about any of 1 mg/kg, 2 mg/kg, 3 mg/kg, 4 mg/kg, 5 mg/kg, 5.5 mg/kg, 5.6 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.2 mg/kg, 11.5 mg/kg, 12 mg/kg, 15 mg/kg, 20 mg/kg, 30 mg/kg, 50 mg/kg, 75 mg/kg, or 100 mg/kg. 4-iodo-3-nitrobenzamide or a metabolite thereof or a pharmaceutically acceptable salt thereof may be administered intravenously, e.g., by IV infusion over about 10 to about 300 minutes, about 30 to about 180 minutes, about 45 to about 120 minutes or about 60 minutes (i.e. about 1 hour). In some embodiments, 4-iodo-3-nitrobenzamide or a metabolite thereof or a pharmaceutically acceptable salt thereof may alternatively be administered orally. In this context, the term “about” has its normal meaning of approximately. In some embodiments, about means ±10% or ±5%.

In some embodiments, 4-iodo-3-nitrobenzamide or a metabolite thereof or a pharmaceutically acceptable salt thereof is administered at about any of 20%, 30%, 40%, 50%, 60%, 70%, 80%, 90%, or 100% of the MTD (“maximum tolerated dose”). In some embodiments, the MTD of 4-iodo-3-nitrobenzamide or a metabolite thereof or a pharmaceutically acceptable salt thereof is in the range of about 1 mg/kg to about 100 mg/kg, about 2 mg/kg to about 50 mg/kg, about 1 to about 25 mg/kg, about 2 to about 70 mg/kg, about 4 to about 100 mg, about 4 to about 25 mg/kg, about 4 to about 20 mg/kg, about 5 to about 15 mg/kg, about 5 to about 10 mg/kg, about 50 to about 100 mg/kg or about 25 to about 75 mg/kg. In some embodiments, the MTD of 4-iodo-3-nitrobenzamide or a metabolite thereof or a pharmaceutically acceptable salt thereof is greater than or at least about any of 1 mg/kg, 2 mg/kg, 3 mg/kg, 4 mg/kg, 5 mg/kg, 6 mg/kg, 7 mg/kg, 8 mg/kg, 8.5 mg/kg, 9 mg/kg, 9.5 mg/kg, 10 mg/kg, 12 mg/kg, or 15 mg/kg. In some embodiments, the MTD of 4-iodo-3-nitrobenzamide or a metabolite thereof or a pharmaceutically acceptable salt thereof is about any of 1 mg/kg, 2 mg/kg, 3 mg/kg, 4 mg/kg, 5 mg/kg, 5.5 mg/kg, 5.6 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.2 mg/kg, 11.5 mg/kg, 12 mg/kg, 15 mg/kg, 20 mg/kg, 30 mg/kg, 50 mg/kg, 75 mg/kg, or 100 mg/kg. MTD for 4-iodo-3-nitrobenzamide or a metabolite thereof or a pharmaceutically acceptable salt thereof may determined by methods known to one skilled in the art.

The dosage of irinotecan may vary depending upon the patient's age, height, weight, overall health, etc. In some embodiments, the dosage of irinotecan is in the range of about 10 mg/m² to about 1000 mg/m², about 25 mg/m² to about 500 mg/m², about 50 mg/m² to about 200 mg/m², about 75 mg/m² to about 200 mg/m², about 75 mg/m² to about 150 mg/m², or about 80 mg/m² to about 125 mg/m². In some embodiments, the dosage of irinotecan is greater than or at least about any of 25 mg/m², 50 mg/m², 75 mg/m², 80 mg/m², 100 mg/m², 125 mg/m², 150 mg/m², 175 mg/m², 200 mg/m², 250 mg/m², or 300 mg/m². In some embodiments, the dosage of irinotecan is about any of 50 mg/m², 75 mg/m², 80 mg/m², 100 mg/m², 125 mg/m², 150 mg/m², 175 mg/m², 200 mg/m², 250 mg/m², or 300 mg/m². Irinotecan may be administered intravenously, e.g. by IV infusion over about 10 to about 500 minutes, about 10 to about 300 minutes, about 30 to about 180 minutes, about 45 to about 120 minutes, about 60 minutes (i.e. about 1 hour), or about 90 minutes. In some embodiments, irinotecan may alternatively be administered orally.

In some embodiments, the MTD of irinotecan is in the range of about 10 mg/m² to about 1000 mg/m², about 25 mg/m² to about 500 mg/m², about 50 mg/m² to about 200 mg/m², about 75 mg/m² to about 200 mg/m². In some embodiments, the MTD of irinotecan is greater than about any of 25 mg/m², 50 mg/m², 75 mg/m², 100 mg/m², 125 mg/m², 150 mg/m², 175 mg/m², 200 mg/m², 250 mg/m², or 300 mg/m². In some embodiments, the MTD of irinotecan is about any of 50 mg/m², 75 mg/m², 80 mg/m², 100 mg/m², 125 mg/m², 150 mg/m², 175 mg/m², 200 mg/m², 250 mg/m², or 300 mg/m². MTD for irinotecan or a pharmaceutically acceptable salt thereof may determined by methods known to one skilled in the art.

In some embodiments, the treatment includes 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, or 15 cycles. Cycle means treatment cycle here. In some embodiments, the treatment includes at most any of 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, or 15 cycles. In some embodiments, the treatment includes at least any of 2 cycles, 3 cycles, 4 cycles, 5 cycles, 6 cycles, 7 cycles, 8 cycles, 9 cycles, or 10 cycles. In some embodiments, the treatment comprises a treatment cycle of at least about any of 1 week, 10 days, 11 days, 2 weeks, 3 weeks, 4 weeks, 30 days, 5 weeks, 6 weeks, 7 weeks, 8 weeks, 9 weeks, 10 weeks, 12 weeks, or 15 weeks. A treatment cycle may be a period of about any of 1 week, 10 days, 11 days, 2 weeks, 3 weeks, 4 weeks, 30 days, 5 weeks, 6 weeks, 7 weeks, 8 weeks, 9 weeks, or 10 weeks. In some embodiments, a treatment cycle is about 11 to about 30 days in length. In some embodiments, the treatment schedule comprises a resting period, wherein neither 4-iodo-3-nitrobenzamide nor irinotecan is administered to the patient. In some embodiments, the resting period is 3 days, 4 days, 5 days, 6 days, 1 week, 10 days, 2 weeks, 3 weeks, 4 weeks, 5 weeks, 6 weeks, 7 weeks, 8 weeks, 9 weeks, 10 weeks, 12 weeks, or 15 weeks.

Administration of (i) 4-iodo-3-nitrobenzamide, a metabolite thereof, or a pharmaceutically acceptable salt thereof; (ii) irinotecan may be on different days of a treatment cycle, such as the treatment cycles described herein. The interval between administration of 4-iodo-3-nitrobenzamide and irinotecan vary within a treatment cycle (e.g., administration is not always spaced apart by 7 day, but may be at intervals of 1 day followed by an interval of 9 days, etc.). Similarly, at certain times during the treatment cycle, 4-iodo-3-nitrobenzamide and irinotecan may be administered at the same time, and at other points during the treatment administered at different times.

4-iodo-3-nitrobenzamide, a metabolite thereof, or a pharmaceutically acceptable salt thereof may be administered every day of the treatment cycle, or administered on certain days but not on every day of the treatment cycle. In some embodiments, 4-iodo-3-nitrobenzamide, a metabolite thereof, or a pharmaceutically acceptable salt thereof is administered daily, once a week, twice a week, three times a week, four times a week, five times a week, six times a week, once 10 days, once two weeks, twice every three weeks, four times every three weeks, once three weeks, once four weeks, once six weeks, or once eight weeks. 4-iodo-3-nitrobenzamide, a metabolite thereof, or a pharmaceutically acceptable salt thereof may be administered on the selected days of each treatment cycle, for example, 4-iodo-3-nitrobenzamide, a metabolite thereof, or a pharmaceutically acceptable salt thereof is administered daily for the period of 2 (or 3, 4, 5, 6, 7, 8, 9, 10, or 11) days of the treatment cycle, and 4-iodo-3-nitrobenzamide, a metabolite thereof, or a pharmaceutically acceptable salt thereof is not administered on other days of the treatment cycle. In some embodiments, 4-iodo-3-nitrobenzamide or the pharmaceutically acceptable salt thereof is administered to said patient. In some embodiments, 4-iodo-3-nitrobenzamide or the pharmaceutically acceptable salt thereof is administered at about 1 mg/kg to about 25 mg/kg. In some embodiments, 4-iodo-3-nitrobenzamide or the pharmaceutically acceptable salt thereof is administered at about 4 mg/kg to about 20 mg/kg. 4-iodo-3-nitrobenzamide (or a metabolite thereof, or a pharmaceutically acceptable salt thereof) may be administered (e.g., at about 5.6 mg/kg) on 4 days of a treatment cycle, e.g., on days 1, 4, 8, 11 of a 21-day treatment cycle. 4-iodo-3-nitrobenzamide (or a metabolite thereof, or a pharmaceutically acceptable salt thereof) may be administered (e.g., at about 11.2 mg/kg) on 2 days of a treatment cycle, e.g., on days 1 and 8 of a 21-day treatment cycle. In some embodiments, 4-iodo-3-nitrobenzamide or the pharmaceutically acceptable salt thereof is administered at about any of 5.6 mg/kg, 8 mg/kg, and 11.2 mg/kg.

In some embodiments, the treatment comprises a treatment cycle of at least 11 days, wherein on days 1, 4, 8 and 11 of the cycle, the patient receives about 10 to about 100 mg/kg of 4-iodo-3-nitrobenzamide or a molar equivalent of a metabolite or pharmaceutically acceptable salt thereof. In some embodiments, the treatment comprises a treatment cycle of at least 11 days, wherein on days 4, 8 and 11 of the cycle, the patient receives about 1 to about 50 mg/kg of 4-iodo-3-nitrobenzamide or a molar equivalent of a metabolite or pharmaceutically acceptable salt thereof. In some embodiments, the treatment comprises a treatment cycle of at least 11 days, wherein on days 1, 4, 8 and 11 of the cycle, the patient receives about 1, 2, 3, 4, 5, 5.6, 6, 7, 8, 9, 10, 11, 11.2, 12, 13, 14, 15, 16, 18, or 20 mg/kg of 4-iodo-3-nitrobenzamide, a metabolite or pharmaceutically acceptable salt thereof.

Irinotecan may be administered daily, e.g., every day of the treatment cycle, or administered on certain days but not on every day of the treatment cycle. In some embodiments, irinotecan is administered daily, once a week, twice a week, twice every 3 weeks, three times a week, four times a week, five times a week, six times a week, once every 10 days, once every two weeks, once every three weeks, once every four weeks, once every six weeks, or once every eight weeks. Irinotecan may be administered on the selected days of each treatment cycle, for example, irinotecan is administered daily on 2 (or 3, 4, 5, 6, 7, 8, 9, 10) days of the treatment cycle, and irinotecan is not administered on other days of the treatment cycle. Irinotecan may be administered (e.g., at about 1000 mg/m²) on 2 days of a treatment cycle, e.g., on days 1 and 8 of a 21-day treatment cycle.

In some embodiments, there is provided a method of treating breast cancer (e.g., metastatic triple negative breast cancer) in a patient, comprising administering to the patient an effective amount of: 4-iodo-3-nitrobenzamide or a metabolite or pharmaceutically acceptable salt thereof and irinotecan or a pharmaceutically acceptable salt thereof. In some embodiments, 4-iodo-3-nitrobenzamide or a metabolite or pharmaceutically acceptable salt thereof is administered at a dose of 1.0-6.0 mg/kg twice weekly (e.g., 5.6 mg/kg on days 1, 4, 8, and 11 of a 21-day treatment cycle). In some embodiments, the effective amount of 4-iodo-3-nitrobenzamide or a metabolite or pharmaceutically acceptable salt thereof is administered at a dose of 1.0-15.0 mg/kg once weekly (e.g., 11.2 mg/kg on days 1 and 8 of a 21-day treatment cycle). In some embodiments, irinotecan is administered at a dose of 50-200 mg/m² once weekly (e.g., 125 mg/m² on days 1 and 8 of a 21-day treatment cycle). In some embodiments, the method comprises at least one cycle, wherein the cycle includes at least 11 days, wherein on days 1 and 8,4-iodo-3-nitrobenzamide or the pharmaceutically acceptable salt thereof is administered at about 11.2 mg/kg, and on days 1 and 8, irinotecan is administered at about 80 mg/m² to about 125 mg/m². In some embodiments, the method comprises a 21-day treatment cycle, wherein 4-iodo-3-nitrobenzamide or a metabolite thereof or a pharmaceutically acceptable salt thereof is administered to the patient at about 5.6 mg/kg on days 1, 4, 8, 11 of the treatment cycle, and wherein irinotecan or a pharmaceutically acceptable salt thereof is administered at about 125 mg/m² on days 1 and 8 of the cycle.

Provided herein are methods of treating locally advanced or metastatic breast cancer in a patient, comprising administering to the patient having locally advanced or metastatic breast cancer an effective amount of (a) 4-iodo-3-nitrobenzamide or a metabolite thereof or a pharmaceutically acceptable salt thereof (e.g., 4-iodo-3-nitrobenzamide), and (b) irinotecan or a pharmaceutically acceptable salt thereof (e.g., irinotecan), wherein the method comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² (e.g., about 125 mg/m²) once weekly for two weeks of the cycle.

In some embodiments, the method comprises at least one cycle, wherein each cycle is a period of 21 days, wherein 4-iodo-3-nitrobenzamide or the pharmaceutically acceptable salt thereof is administered at about 8 mg/kg twice weekly for two weeks of the cycle, and wherein irinotecan is administered at about 80 mg/m² to about 125 mg/m² twice the cycle (e.g., once weekly for two weeks of the cycle). In some embodiments, the method comprises at least one cycle, wherein each cycle is a period of 21 days, wherein 4-iodo-3-nitrobenzamide or the pharmaceutically acceptable salt thereof is administered at about 8 mg/kg twice weekly for two weeks of the cycle (e.g., on days 1, 4, 8 and 11), and wherein irinotecan or a pharmaceutically acceptable salt thereof (e.g., irinotecan) is administered at about 80 mg/m² to about 125 mg/m² weekly for two weeks of the cycle (e.g., on days 1 and 8). In some embodiments, the method comprises at least one cycle, wherein each cycle is a period of 21 days, wherein 4-iodo-3-nitrobenzamide or the pharmaceutically acceptable salt thereof is administered at about 8 mg/kg twice weekly for the first two weeks of the cycle (e.g., on days 1, 4, 8 and 11), wherein irinotecan is administered at about 80 mg/m² to about 125 mg/m² weekly for the first two weeks of the cycle (e.g., on days 1 and 8), wherein neither 4-iodo-3-nitrobenzamide (or the pharmaceutically acceptable salt thereof) nor irinotecan is administered during the third week of the cycle. For example, in some embodiments, 4-iodo-3-nitrobenzamide or the pharmaceutically acceptable salt thereof is administered at about 8 mg/kg on days 1, 4, 8, and 11 of the 21-day cycle, and irinotecan or a pharmaceutically acceptable salt thereof is administered at about 125 mg/m² on days 1 and 8 of the 21-day cycle.

In some embodiments, the method comprises at least one cycle, wherein the cycle includes at least 11 days, wherein on days 1 and 8,4-iodo-3-nitrobenzamide or the pharmaceutically acceptable salt thereof is administered at about 11.2 mg/kg, and on days 1 and 8, irinotecan is administered at about 80 mg/m² to about 125 mg/m². In some embodiments, the method comprises at least one cycle, wherein the cycle includes at least 11 days, wherein 4-iodo-3-nitrobenzamide or the pharmaceutically acceptable salt thereof is administered at about 11.2 mg/kg once weekly for two weeks of the cycle, and wherein irinotecan is administered at about 125 mg/m² once weekly for two weeks of the cycle. In some embodiments, the method comprises at least one cycle, wherein the cycle is a period of 21 days, wherein 4-iodo-3-nitrobenzamide or the pharmaceutically acceptable salt thereof is administered at about 11.2 mg/kg once weekly for two weeks of the cycle, and wherein irinotecan is administered at about 80 mg/m² to about 125 mg/m² once weekly for two weeks of the cycle. In some embodiments, the method comprises at least one cycle, wherein the cycle is a period of 21 days, wherein 4-iodo-3-nitrobenzamide or the pharmaceutically acceptable salt thereof is administered at about 11.2 mg/kg once weekly for two weeks of the cycle, and wherein irinotecan is administered at about 125 mg/m² once weekly for two weeks of the cycle. In some embodiments, the method comprises at least one cycle, wherein the cycle is a period of 21 days, wherein on days 1 and 8,4-iodo-3-nitrobenzamide or the pharmaceutically acceptable salt thereof is administered at about 11.2 mg/kg and irinotecan is administered at about 125 mg/m².

4-iodo-3-nitrobenzamide (or a metabolite thereof) and irinotecan may be co-administered to a patient. Co-administration is meant to include simultaneous or sequential administration of the compounds individually or in combination (more than one compound), such as described herein.

Simultaneous administration in this context means that a first compound and second compound are administered with a time separation of no more than about 15 minutes, such as no more than about any of 10, 5, or 1 minutes. When the first compound and second compound are administered simultaneously, the first compound and second compound may be contained in the same composition (e.g., a composition comprising both first compound and second compound) or in separate compositions (e.g., a first compound in one composition and a second compound is contained in another composition). The first compound described herein may be 4-iodo-3-nitrobenzamide (or pharmaceutically acceptable salt or solvate thereof, or metabolite thereof) and the second compound may be irinotecan. Or, the first compound may be irinotecan and the second compound may be 4-iodo-3-nitrobenzamide (or pharmaceutically acceptable salt or solvate thereof, or metabolite thereof).

Sequential administration described herein means that a first compound and second compound are administered with a time separation of more than about 15 minutes, such as more than about any of 20, 30, 40, 50, 60, or more minutes. Either the first compound or the second compound may be administered first. The first compound and second compound are contained in separate compositions, which may be contained in the same or different packages or kits. The first compound described herein may be 4-iodo-3-nitrobenzamide (or pharmaceutically acceptable salt or solvate thereof, or metabolite thereof) and the second compound may be irinotecan. Or, the first compound may be irinotecan and the second compound may be 4-iodo-3-nitrobenzamide (or pharmaceutically acceptable salt or solvate thereof, or metabolite thereof).

4-iodo-3-nitrobenzamide (or pharmaceutically acceptable salt or solvate thereof, or metabolite thereof) and/or irinotecan may be continuously or not continuously given to a patient. “Not continuously” means that the compound or composition provided herein is not administered to the patient over a period of time, e.g., there is a resting period when the patient does not receive the compound or composition. It may be that one compound is administered continuously administered to a patient while the second compound is not administered continuously administered to the patient.

In some cases, a beneficial effect is achieved when the administration of irinotecan is temporally removed from the administration of the 4-iodo-3-nitrobenazmide (or pharmaceutically acceptable salt or solvate thereof, or metabolite thereof) by a significant period of time (e.g., about 12 hours, about 24 hours, about 36 hours, about 48 hours, etc.). Or, for example, administration is spaced apart by at least 1 day, 2 days, 3 days, 4 days, 5 days, 6 days, 7 days, 8 days, 9 days, 10 days, etc.). For example, administration on different days of a treatment cycle, such as the treatment cycles described herein. The interval between administration of the 4-iodo-3-nitrobenzamide and irinotecan may vary within a treatment cycle (e.g., administration is not always spaced apart by 1 day, but may be intervals of 1 day followed by an interval of 3 days, etc.). Similarly, at certain times during the treatment cycle, the 4-iodo-3-nitrobenzamide and irinotecan may be administered at the same time, and at other points during the treatment administered at different times.

4-iodo-3-nitrobenzamide (or a metabolite thereof or a pharmaceutically acceptable salt thereof) and irinotecan (or a pharmaceutically acceptable salt thereof) may be formulated in separate formulations or in the same formulation. 4-iodo-3-nitrobenzamide (or a metabolite thereof or a pharmaceutically acceptable salt thereof) and irinotecan (or a pharmaceutically acceptable salt thereof) may be administered through different administration route or using same administration routes. In some embodiments, there are provided formulations (e.g., pharmaceutical formulations) comprising 4-iodo-3-nitrobenzamide (or a metabolite thereof, or a pharmaceutically acceptable salt thereof) and irinotecan (or a pharmaceutically acceptable salt thereof), and a carrier, such as a pharmaceutically acceptable carrier. The formulations may include optical isomers, diastereomers, carriers of the compounds disclosed herein. In some embodiments, the carrier is a cyclodextrin, or a derivative thereof, e.g., hydroxypropyl-B-cyclodextrin (HPBCD). In some embodiments the formulations are formulated for intravenous administration.

A formulation may comprise both the 4-iodo-3-nitrobenzamide compound and acid forms in particular proportions, depending on the relative potencies of each and the intended indication. The two forms may be formulated together or in different formulations. They may be in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

The pharmaceutical compositions of the present invention may be provided as a prodrug and/or may be allowed to interconvert to 4-iodo-3-nitrobenzamide form in vivo after administration. That is, either 4-iodo-3-nitrobenzamide or metabolites thereof or pharmaceutically acceptable salts may be used in developing a formulation for use in the present invention.

Also provided herein are synergistic compositions used for treating breast cancer (e.g., metastatic breast cancer) in a patient comprising a) 4-iodo-3-nitrobenzamide, or a metabolite thereof, or a pharmaceutically acceptable salt or solvate thereof, b) irinotecan (or a pharmaceutically acceptable salt thereof).

The pharmaceutical compositions of the 4-iodo-3-nitrobenzamide (or pharmaceutically acceptable salt or solvate thereof, or metabolite thereof) and irinotecan can be combined with other active ingredients, such as other chemotherapeutic agents as described herein. The two compounds and/or forms of a compound may be formulated together, in the same dosage unit e.g., in one cream, suppository, tablet, capsule, or packet of powder to be dissolved in a beverage; or each form may be formulated in separate units, e.g., two creams, two suppositories, two tablets, two capsules, a tablet and a liquid for dissolving the tablet, a packet of powder and a liquid for dissolving the powder, etc.

In some embodiments, the composition is administered in unit dosage form. In some embodiments, the unit dosage form is adapted for oral or parenteral administration. In some embodiments, upon administration of the composition, at least one therapeutic effect is obtained, said at least one therapeutic effect being reduction in size of a tumor, reduction in metastasis, complete remission, partial remission, pathologic complete response, or stable disease. In some embodiments, upon administration of the composition, an improvement of clinical benefit rate (“CBR”) is obtained as compared to treatment with irinotecan but without 4-iodo-3-nitrobenzamide or the metabolite thereof or the pharmaceutically acceptable salt thereof. In some embodiments, the improvement of clinical benefit rate is at least about any of 20%, 30%, 40%, 50%, 60%, 70%, 80%, or 90%. In some embodiments, CBR=CR+PR+SD≧6 months. In some embodiments, CBR=CR+PR+SD≧6 cycles.

In some embodiments, 4-iodo-3-nitrobenzamide or a metabolite thereof or a pharmaceutically acceptable salt thereof is administered intravenously. In some embodiments, irinotecan or a pharmaceutically acceptable salt thereof is administered intravenously.

For injection, the 4-iodo-3-nitrobenzamide or pharmaceutically acceptable salt thereof may be formulated for administration in aqueous solutions, preferably in physiologically compatible buffers such as phosphate buffers, Hank's solution, or Ringer's solution. Such compositions may also include one or more excipients, for example, preservatives, solubilizers, fillers, lubricants, stabilizers, albumin, and the like. Formulations of 4-iodo-3-nitrobenzamide are described in US Pat. Publ. No. 2008/0176946 A1, which is incorporated by reference in its entirety, particularly with reference to intravenous (e.g., hydroxypropyl-β-cyclodextrin, etc.) and oral (e.g., sodium lauryl sulfate, etc.) formulations. In some embodiments, 4-iodo-3-nitrobenzamide is formulated in 25% (w/v) hydroxypropyl-β-cyclodextrin and 10 mM phosphate buffer for intravenous administration as described in U.S. Patent Application Publication No. 2010/0160442, which is incorporated herein by reference.

Additional methods of formulation, such as for irinotecan (or a pharmaceutically acceptable salt thereof), are known in the art, for example, as disclosed in Remington's Pharmaceutical Sciences, latest edition, Mack Publishing Co., Easton, Pa. Compositions described herein may also be formulated for transmucosal administration, buccal administration, for administration by inhalation, for parental administration, for transdermal administration, and rectal administration.

Compositions described herein may also be formulated for transmucosal administration, buccal administration, for administration by inhalation, for parental administration, for transdermal administration, and rectal administration.

Typical salts for compositions, formulations, and methods provided herein may be those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, where compounds contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine. In some embodiments, 4-iodo-3-nitrobenzamide is formulated in 25% (w/v) hydroxypropyl-β-cyclodextrin and 10 mM phosphate buffer for intravenous administration as described in U.S. Patent Application Publication No. 2010/0160442, which is incorporated herein by reference.

Pharmaceutical compositions suitable for use as described herein include compositions wherein the active ingredients are present in an effective amount, i.e., in an amount effective to achieve therapeutic and/or prophylactic benefit in a breast cancer (e.g., metastatic breast cancer) described herein. The actual amount effective for a particular administration will depend on the breast cancer (e.g., metastatic breast cancer) being treated, the condition of the individual, the formulation, and the route of administration, as well as other factors known to those of skill in the art in view of the specific teaching provided herein. In light of the disclosure herein, optimization of an effective amount of 4-iodo-3-nitrobenzamide or a metabolite thereof or a pharmaceutically acceptable salt thereof and irinotecan or a pharmaceutically acceptable salt thereof, within the ranges specified, may be determined.

The compositions described herein may be administered to a patient through appropriate route, such as, but are not limited to intravenous, intra-arterial, intraperitoneal, intrapulmonary, inhalation, intravesicular, intramuscular, intra-tracheal, subcutaneous, intraocular, intrathecal, transmucosal, transdermal, intranasal, epidural, and oral routes. In some embodiments, the composition or compound(s) provided herein is administered by the parenteral route, e.g., intravenously, intraperitoneally, subcutaneously, intradermally, or intramuscularly. In some embodiments, sustained continuous release of the formulations or compositions described herein are administered.

Compositions provided herein may also be administered by a convenient route, for example by infusion or bolus injection, by absorption through epithelial or mucocutaneous linings (e.g., oral mucosa, rectal and intestinal mucosa, etc.) and may be administered in combination with other biologically active agents, e.g., such as described herein. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

Kits, uses, articles of manufacture

Provided herein are kits for administration of 4-iodo-3-nitrobenzamide or a metabolite thereof or a pharmaceutically acceptable salt thereof, and irinotecan. In certain embodiments the kits may include a dosage amount of at least one composition as disclosed herein. Kits may further comprise suitable packaging and/or instructions for use of the formulation. Kits may also comprise a means for the delivery of the formulation thereof.

In some embodiments, there is provided a kit comprising (a) 4-iodo-3-nitrobenzamide or a metabolite thereof or a pharmaceutically acceptable salt thereof and (b) irinotecan or a pharmaceutically acceptable salt thereof. In some embodiments, the kit further comprises instructions (e.g., instructions on package insert, product insert or label) for using (a) 4-iodo-3-nitrobenzamide or a metabolite thereof or a pharmaceutically acceptable salt thereof and (b) irinotecan to treat locally advanced or metastatic breast cancer in a patient according to a method provided herein. In some embodiments, there is provide a kit comprising (i) 4-iodo-3-nitrobenzamide or a metabolite thereof or a pharmaceutically acceptable salt thereof and (ii) instructions (e.g., instructions on package insert, product insert or label) for using (a) 4-iodo-3-nitrobenzamide or a metabolite thereof or a pharmaceutically acceptable salt thereof and (b) irinotecan to treat locally advanced or metastatic breast cancer in a patient according to a method provided herein. A kit described herein may comprise packaging.

In some embodiments, there is provided a kit comprising (a) 4-iodo-3-nitrobenzamide or a metabolite thereof or a pharmaceutically acceptable salt thereof and (b) irinotecan or a pharmaceutically acceptable salt thereof. In some embodiments, the kit further comprises instructions (e.g., instructions on package insert, product insert, or label) for using (a) 4-iodo-3-nitrobenzamide or a metabolite thereof or a pharmaceutically acceptable salt thereof and (b) irinotecan or a pharmaceutically acceptable salt thereof to treat metastatic breast cancer brain metastasis (e.g., brain metastasis measuring at least about or larger than about 0.5 centimeter) according to a method described herein. Also provided herein are kits comprising (i) 4-iodo-3-nitrobenzamide or a metabolite thereof or a pharmaceutically acceptable salt thereof and (ii) instructions (e.g., instructions on package insert, product insert, or label) for using (a) 4-iodo-3-nitrobenzamide or a metabolite thereof or a pharmaceutically acceptable salt thereof and (b) irinotecan or a pharmaceutically acceptable salt thereof to treat metastatic breast cancer brain metastasis (e.g., brain metastasis measuring at least about or larger than about 0.5 centimeter) in a patient according to a method described herein. In some embodiments, the breast cancer is ER-negative, PR-negative, and HER2-nonoverexpressing. In some embodiments, the brain metastasis is at least about or larger than about 0.5 centimeter (e.g., brain metastasis measuring at least about or larger than about 0.5 centimeter in longest dimension). The dosage or treatment regimen for (a) 4-iodo-3-nitrobenzamide or a metabolite thereof or a pharmaceutically acceptable salt thereof and/or (b) irinotecan may be included in the instructions of any of the kits described herein and may be according to any of the dosages or treatment regimens described herein.

The kits may optionally include appropriate instructions for preparation and administration of the composition, side effects of the composition, and any other relevant information. The instructions may be in any suitable format, including, but not limited to, printed matter, videotape, computer readable disk, optical disc or directions to internet-based instructions.

For example, in some embodiments, there is provided a kit for treating a patient with breast cancer brain metastasis comprising (a) 4-iodo-3-nitrobenzamide or a metabolite thereof or a pharmaceutically acceptable salt thereof, and (b) irinotecan or a pharmaceutically acceptable salt thereof, wherein the breast cancer is ER-negative, PR-negative, and HER2-nonoverexpressing. In some embodiments, the kit further comprises instructions for using effective amount of (a) 4-iodo-3-nitrobenzamide or a metabolite thereof or a pharmaceutically acceptable salt thereof, and (b) irinotecan or a pharmaceutically acceptable salt thereof to treat the patient with breast cancer brain metastasis. In some embodiments, there is provided a kit comprising (a) 4-iodo-3-nitrobenzamide or a metabolite thereof or a pharmaceutically acceptable salt thereof, and (b) instructions for using an effective amount of 4-iodo-3-nitrobenzamide or a metabolite thereof or a pharmaceutically acceptable salt thereof, in combination with irinotecan or a pharmaceutically acceptable salt thereof to treat a patient with breast cancer brain metastasis, wherein the breast cancer is ER-negative, PR-negative, and HER2-nonoverexpressing. In some embodiments, there is provided a kit comprising (a) 4-iodo-3-nitrobenzamide or a metabolite thereof or a pharmaceutically acceptable salt thereof, (b) irinotecan or a pharmaceutically acceptable salt thereof, and (c) instructions for using 4-iodo-3-nitrobenzamide or a metabolite thereof or a pharmaceutically acceptable salt thereof, and irinotecan or a pharmaceutically acceptable salt thereof to treat locally advanced or metastatic breast cancer in a patient, wherein the treatment comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle. In some embodiments, there is provided a kit comprising (a) 4-iodo-3-nitrobenzamide or a metabolite thereof or a pharmaceutically acceptable salt thereof, and (b) instructions for using 4-iodo-3-nitrobenzamide or a metabolite thereof or a pharmaceutically acceptable salt thereof, in combination with irinotecan or a pharmaceutically acceptable salt thereof to treat locally advanced or metastatic breast cancer in a patient, wherein the treatment comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle.

The kits may include other pharmaceutical agents (such as the side-effect limiting agents, chemotherapy agents, gene therapy agents, DNA therapy agents, RNA therapy agents, viral therapy agents, nanotherapy agents, small molecule enzymatic inhibitors, anti-metastatic agents, etc.), for use in conjunction with 4-iodo-3-nitrobenzamide or a metabolite thereof or a pharmaceutically acceptable salt thereof, and irinotecan or a pharmaceutically acceptable salt thereof. These agents may be provided in a separate form, or mixed with 4-iodo-3-nitrobenzamide or a metabolite thereof or a pharmaceutically acceptable salt thereof, and irinotecan, provided such mixing does not reduce the effectiveness of 4-iodo-3-nitrobenzamide (or a metabolite thereof or a pharmaceutically acceptable salt thereof) or irinotecan or a pharmaceutically acceptable salt thereof, and is compatible with the route of administration. Similarly the kits may include additional agents for adjunctive therapy or other agents known to the skilled artisan as effective in the treatment or prevention of breast cancer (e.g., metastatic breast cancer) described herein.

In another aspect, provided are kits for treating a patient who suffers from or is susceptible to the breast cancer (e.g., locally advanced metastatic breast cancer) described herein, comprising a first container comprising a dosage amount of a formulation as disclosed herein, and instructions for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the composition to be administered to the patient.

Kits may also be provided that contain sufficient dosages of 4-iodo-3-nitrobenzamide or a metabolite thereof or a pharmaceutically acceptable salt thereof, and/or irinotecan or a pharmaceutically acceptable salt thereof (including formulation thereof) as disclosed herein to provide effective treatment for a patient for an extended period, such as 1-3 days, 1-5 days, a week, 2 weeks, 3 weeks, 4 weeks, 6 weeks, 8 weeks, 3 months, 4 months, 5 months, 6 months, 7 months, 8 months, 9 months or more. Kits may also include multiple doses of the compounds and instructions for use and packaged in quantities sufficient for storage and use in pharmacies, for example, hospital pharmacies and compounding pharmacies.

The kits may include the compounds as described herein packaged in either a unit dosage form or in a multi-use form. The kits may also include multiple units of the unit dose form. In certain embodiments, provided are the compound described herein in a unit dose form. In other embodiments the compositions may be provided in a multi-dose form (e.g., a blister pack, etc.).

Also provided are medicines for treating breast cancer (e.g., locally advanced metastatic breast cancer). In some embodiments, the medicine comprises a) a composition comprising 4-iodo-3-nitrobenzamide, or a metabolite thereof, or a pharmaceutically acceptable salt thereof, and irinotecan. 4-iodo-3-nitrobenzamide (or a metabolite thereof, or a pharmaceutically acceptable salt thereof) and irinotecan can be present in separate containers or in a single container. It is understood that the medicine may comprise one distinct composition or two or more compositions wherein one composition comprises 4-iodo-3-nitrobenzamide (or a metabolite thereof, or a pharmaceutically acceptable salt thereof) and one composition comprises irinotecan.

Also provided herein are uses of 4-iodo-3-nitrobenzamide, a metabolite thereof, or a pharmaceutically acceptable salt or solvate thereof, in combination with irinotecan, a pharmaceutically acceptable salt or solvate thereof for the manufacture of a medicament for the treatment or prevention of breast cancer (e.g., locally advanced breast cancer, metastatic breast cancer, breast cancer brain metastasis, or ER-negative, PR-negative, and HER2-nonoverexpressing breast cancer brain metastasis) described herein. Also provided herein are uses of 4-iodo-3-nitrobenzamide, a metabolite thereof, or a pharmaceutically acceptable salt or solvate thereof for the manufacture of a medicament for the treatment or prevention of breast cancer (e.g., locally advanced breast cancer, metastatic breast cancer, breast cancer brain metastasis, or ER-negative, PR-negative, and HER2-nonoverexpressing breast cancer brain metastasis) in combination with irinotecan, a pharmaceutically acceptable salt or solvate thereof described herein. In certain embodiments, the medicament is provided for the treatment of breast cancer (e.g., locally advanced breast cancer, metastatic breast cancer, breast cancer brain metastasis, or ER-negative, PR-negative, and HER2-nonoverexpressing breast cancer brain metastasis). Also provided herein are uses of -iodo-3-nitrobenzamide, a metabolite thereof, or a pharmaceutically acceptable salt or solvate thereof, in combination with irinotecan, a pharmaceutically acceptable salt or solvate thereof, for treatment of breast cancer (e.g., locally advanced breast cancer, metastatic breast cancer, breast cancer brain metastasis, or ER-negative, PR-negative, and HER2-nonoverexpressing breast cancer brain metastasis) in a patient. Also provided herein are synergistic compositions used for treating breast cancer (e.g., locally advanced breast cancer, metastatic breast cancer, breast cancer brain metastasis, or ER-negative, PR-negative, and HER2-nonoverexpressing breast cancer brain metastasis) in a patient comprising a) 4-iodo-3-nitrobenzamide, or a metabolite thereof, or a pharmaceutically acceptable salt or solvate thereof, and b) irinotecan, or pharmaceutically acceptable salt or solvate thereof, to said patient. The uses described herein may in accordance with any of the methods provided herein.

Also provided are articles of manufacture comprising the compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed.

### EXAMPLES

The examples below are intended to be purely exemplary of the invention and should therefore not be considered to limit the invention in any way. The following examples and detailed description are offered by way of illustration and not by way of limitation.

### Example 1

### A Phase 1/1b Dose Escalation Study Evaluating 4-iodo-3-nitrobenzamide (BA) as a Single Agent and in Combination with Irinotecan in Subjects with Advanced Solid Tumors

This study includes treatment of advanced solid tumors including locally advanced or metastatic breast cancer. The study has two phases: phase 1 and phase 1b.

### Objectives

The primary objectives of phase 1 are to assess the safety, pharmacokinetics and to determine the maximum tolerated dose (MTD) of 4-iodo-3-nitrobenzamide as a single agent in patients with advanced solid tumors that are refractory to standard therapy. The primary objectives of phase 1b are to determine safety and maximum tolerated dose of 4-iodo-3-nitrobenzamide in combination with irinotecan in patients with locally advanced or metastatic breast cancer and to investigate the effect of this maximum tolerated dose of 4-iodo-3-nitrobenzamide in combination with irinotecan in patients with locally advanced or metastatic breast cancer. The secondary objectives are to assess safety profiles such as significant laboratory changes and adverse events (AEs) not defined as a dose limiting toxicity (DLT). The exploratory objectives include: to study whether BRCA activity is down-regulated in metastatic breast cancer; to quantitatively measure total cellular levels of topoisomerase I as well as levels within the nucleus and cytoplasm in order to determine a nuclear to cytoplasmic ratio, which may be predictive of response to inhibitors of topoisomerase I.

Study design

Phase 1: 4-iodo-3-nitrobenzamide is administered intravenously twice weekly (days 1 and 4 of each week) for 3 weeks, followed by a one week 4-iodo-3-nitrobenzamide treatment-free period per one 28-day cycle. Cycle one (day 1 through day 28) is defined as the Safety Phase of the study during which DLTs is assessed and the MTD is determined. The remainder of the study is termed the Maintenance Phase. A subject may participate in this study until he/she experiences a drug intolerance or disease progression. The first assessment of tumor response is performed during week 8 of this portion of the study, and approximately every 8 weeks thereafter. The modified Response Evaluation Criteria in Solid Tumors (RECIST) is used to establish disease progression. For non-measurable disease, best medical practices are used to determine disease progression.

In cohort A (starting dose), a single subject receives 4-iodo-3-nitrobenzamide twice weekly at a dose level of 0.5 mg/kg based on weight measured at screening. If this subject experiences a grade 2 toxicity or higher, then 3 additional subjects would be enrolled in this cohort (only 2 additional subjects would be enrolled in this cohort if the initial subject receives at least 80% of the planned cycle 1 doses). If no additional subjects dosed in this cohort experience a DLT, then dose escalation would occur as below. If no DLT occurs in the initial subject, dose escalation would occur as below. The safety review group has the ability to recommend dose escalation (opening of a new cohort), without expansion with additional subjects, for cases of grade 2 toxicity seen in a single subject that are deemed not to be clinically relevant and/or study treatment-related. Dose Escalation Prior to Grade 2 Toxicity (Potential Cohorts B-J): Until a subject experiences a grade 2 toxicity or higher, one subject would be initially enrolled in all subsequent cohorts at planned 100% dose level increases, with possible cohort expansion as described for cohort A. Safety data are reviewed after 6 doses of 4-iodo-3-nitrobenzamide, and a decision to escalate to the next cohort would be made if no subject experiences a grade 2 toxicity or higher. If 1 subject in this cohort experiences a grade 2 toxicity or higher, then 3 additional subjects would be enrolled in this cohort (only 2 additional subjects would be enrolled in this cohort if the initial subject receives at least 80% of the planned cycle 1 doses). If none of these three additional subjects dosed in this cohort experience a DLT, then further dose escalation would occur. If 1 of 3 subjects experience a DLT, then 3 additional subjects would be enrolled in the same cohort with the same dose. If 0 of these 3 subjects experience a DLT then escalation would occur. If one or more of the additional subjects in a cohort experience a DLT, then the previous lower dose level would be defined as the MTD. Additional subjects may be accrued at the MTD if needed to ensure at least 18 subjects receive 4-iodo-3-nitrobenzamide in the Phase 1 portion of the study. Dose Escalation After Grade 2 Toxicity Level (Potential Cohorts B-J): After the dose associated with the initial grade 2 toxicity is expanded and cleared for dose escalation to the next level, then three subjects would be initially enrolled in all future cohorts (Cohorts B, C, D, E, F, G, H, I, or J). If 0 of the 3 initial subjects experience a DLT, then dose escalation to the next cohort would proceed. If 1 of 3 subjects experience a DLT, then 3 additional subjects would be enrolled in the same cohort with the same dose. If 0 of these 3 subjects experience a DLT then escalation would occur. If one or more of the additional subjects in a cohort experience a DLT, then the previous lower dose level would be defined as the MTD. Additional subjects may be accrued at the MTD if needed to ensure that at least 18 subjects receive 4-iodo-3-nitrobenzamide in the Phase 1 portion of the study. Overall Dose Escalation Limitations: When a grade 2 toxicity has been observed and that dose level subsequently cleared, individual dose escalations between cohorts would be more conservative, and would be limited to approximately a maximum 40% increase from the previous dose level until a grade 3 toxicity is seen, with subsequent escalations limited to approximately 25% dose increases. See Table 1. Absolute dose escalation is decided by the safety review group after review of all available data.

TABLE 1

| Toxicity at a given dose level | Dosage increment for next cohort |
|---|---|
| Grade 0-1 | 100% |
| Grade 2 | 40% |
| Grade >=3 toxicity | 25% |

Maximum Tolerated Dose (MTD) is defined as the highest dose level with an observed incidence of a DLT in <33% of the subjects enrolled in the cohort. Dose Limiting Toxicity (DLT) is defined as a grade 3, or 4, severe hematological or non-hematological toxicity thought to be possibly due to study drug, during the initial 28 days of study except fatigue, nausea, diarrhea, vomiting, neutropenia, febrile neutropenia, thrombocytopenia, anemia, AST, and ALT, which would be defined as: Grade 3 fatigue, persistent for more than 7 days; Grade 3 nausea, diarrhea, and/or vomiting despite maximum supportive care, or any Grade 4 nausea, diarrhea, and/or vomiting; Grade 4 neutropenia (ANC<0.5×10⁹/L) for more than 5 days; Grade 4 thrombocytopenia (platelet count<25.0×10⁹/L); Grade 4 anemia; Grade 2 neurotoxicity; AST or ALT>5×ULN. Subjects that require GCF and/or platelet support should be considered as having a DLT.

Phase 1b: Phase 1b enrollment proceeds in cohorts of 3 patients with locally advanced or metastatic breast cancer in accordance with standard Phase I dose escalation rules (3+3). No intra-subject dose escalation is allowed in this portion of the study. Irinotecan is administered intravenously over 90 minutes on day 1 and then on day 8, beginning at a dose of 80 mg/m², and escalating up to 125 mg/m². 4-iodo-3-nitrobenzamide is administered intravenously over 60 minutes on day 1 beginning immediately after completion of the irinotecan infusion, and again on day 4, at a dose of 8.0 mg/kg. This twice-weekly dosing is repeated over a total of 2 weeks. Week 3 is a rest period in which neither irinotecan nor 4-iodo-3-nitrobenzamide is dosed. The dose of 4-iodo-3-nitrobenzamide selected for the phase 1b portion of the study is partially based on clearance of the 8.0 mg/kg dose level in the phase 1 portion of the study. This overall three week period is defined as one study cycle and is repeated every three weeks. Also, the initial three week cycle (Cycle 1) is the basis for decisions regarding tolerability and dose escalation to the next cohort. Dose escalation (or reduction) is done following guidance in Table 2 starting at dose level 1b-1.

TABLE 2

| | | 4-iodo-3-nitrobenzamide |
|---|---|---|
| Phase 1b cohort/Level | Irinotecan Dose | Dose |
| 1b-0 | 60 mg/m² | 8.0 mg/kg |
| 1b-1 (initial dose level) | 80 mg/m² | 8.0 mg/kg |
| 1b-2 | 100 mg/m² | 8.0 mg/kg |
| 1b-3 | 125 mg/m² | 8.0 mg/kg |

Once the highest well tolerated combination is identified, that dose would be studied in an additional 18 subjects with metastatic breast cancer. During accrual in this latter group, subjects continue to be monitored for toxicity. If >6 of 18 subjects experience grade 3 or 4 drug related DLT (as defined in the protocol), the dosing of study drugs would be reassessed and the protocol would either be terminated or amended to investigate a less toxic dose and/or schedule.

The first assessment of tumor response is performed after week 6 (completion of 2 cycles of therapy), and then after every 6 weeks in addition to the initial staging done at baseline. The modified Response Evaluation Criteria in Solid Tumors (RECIST) is used to establish disease response or progression. Subjects may participate in this study until a subject experiences a drug intolerance or disease progression. Subjects with complete regression (CR) receive 4 additional treatment cycles, and subjects with partial regression (PR) or stable disease (SD) may continue therapy indefinitely, at the investigator's discretion.

Maximum Tolerated Dose (MTD) is defined as the highest dose level with an observed incidence of a DLT in <33% of the subjects enrolled in the cohort. Dose Limiting Toxicity (DLT) is defined as any grade 3 or 4, severe hematological or non-hematological toxicity, thought possibly due to irinotecan or potentiated by 4-iodo-3-nitrobenzamide, during the initial 21 days of study except fatigue, nausea, diarrhea, vomiting, neutropenia, febrile neutropenia, thrombocytopenia, anemia, hypertension, AST, and ALT, which would be defined as: Grade 3 fatigue, persistent for more than 7 days; Grade 3 or 4 nausea, diarrhea, and/or vomiting despite maximum supportive care; Grade 3 or 4 neutropenia with fever >38.50C; Grade 4 neutropenia (ANC<0.5×10⁹/L) for more than 7 days; Grade 4 thrombocytopenia (platelet count<25.0×10⁹/L); Grade 4 hypertension despite anti-hypertensives; AST or ALT >2 grade change from baseline.

The overall study is planned for as many as 6 cycles of 4-iodo-3-nitrobenzamide treatment (with or without irinotecan) per subject at the MTD level and as many as 12 cycles of total 4-iodo-3-nitrobenzamide treatment, dependent on the date of subject study enrollment. In the event that the study subject(s) demonstrate persistent clinically favorable response that is accompanied by documented radiographic response (complete response, partial response, and stable disease without any tumor growth), the sponsor would, if possible, maintain such subject(s) on 4-iodo-3-nitrobenzamide treatment. It is anticipated that each subject would be on the study for 2 weeks of screening, for as long as 48 weeks of treatment, and 30 days of follow-up.

### Endpoints

Phase 1—Primary endpoints: safety/tolerability to characterize DLT; PK profiles: 4-iodo-3-nitrobenzamide half life (t½), maximum observed concentration (Cₘₐₓ), area under the plasma concentration-time curve (AUC), and clearance (CL). Secondary endpoints: Tumor response per RECIST criteria; safety profiles: significant laboratory changes and other AEs (not defined as a DLT). Exploratory endpoints include reduction in circulating tumor cell (CTC) levels.

Phase 1b—Primary endpoints: Determination of the safety and tolerability of the combination of irinotecan plus 4-iodo-3-nitrobenzamide; Determination of the effect of this combination in subjects with locally advanced or metastatic breast cancer by Objective Response Rate (ORR; PR+CR). Secondary endpoints: Determination of the effect of this combination in subjects with metastatic breast cancer by Clinical Benefit Rate (CBR; ORR+stable disease). Exploratory points include: To study the formation of double strand DNA breaks and homologous recombination in pre- and post-treatment tumor tissues by staining r-H2AX foci and RAD51 foci; To study the status of BRCA on response in subjects with metastatic breast cancer; To study the expression of hypoxia markers in metastatic breast cancer; To quantitatively measure total cellular levels of topoisomerase I as well as levels within the nucleus and cytoplasm in order to determine a nuclear to cytoplasmic ratio, which may be predictive of response to inhibitors of topoisomerase I.

### Subject Eligibility Criteria

Inclusion criteria include the following: ≧18 years old with a pathologically documented, advanced solid tumor that is refractory to standard treatment or for which no standard therapy is available (phase 1 only); ≧18 years old with a histologically documented, adenocarcinoma of the breast with progressing locally advanced or metastatic disease and at least one bi-dimensionally measurable indicator lesion of at least 2.0 cm as assessed by computed tomography, magnetic resonance imaging, or ultra-sonography (phase 1b only); Prior treatment with at least one regimen containing an anthracycline, an anthraquinone, a taxane, or doxorubicin is required (phase 1b only); Maximum of one adjuvant regimen and two regimens for metastatic disease, whether or not all of these were based on anthracycline or taxane (phase 1b only); Eastern Oncology Cooperative Group (ECOG) performance status of ≦2; Completion of prior chemotherapy at least 3 weeks prior to trial entry and recovery from toxicity of prior chemotherapy; Radiation therapy must be completed at least 3 weeks prior to trial entry, and radiated lesions may not serve as target lesions; Subjects may have CNS metastases if individual does not require steroids, whole brain XRT, or gamma/cyber knife, and brain metastases are clinically stable without symptomatic progression; Adequate organ function defined as: ANC≧1,500/mm³, platelets ≧100,000/mm³, creatinine clearance >50 mL/min, ALT and AST <2.5× upper limit of normal (ULN) (or <5×ULN in case of liver metastases); total bilirubin <1.5 mg/dL; Tissue block (primary or metastatic) available for pharmacogenomic studies is recommended, although its absence would not exclude subjects from participating; Women of child-bearing potential must have documented negative pregnancy test within two weeks of trial entry and agree to use acceptable birth control during the duration of the trial therapy; Signed, IRB-approved written informed consent.

Exclusion criteria include the following: Lesions identifiable only by PET; Major medical conditions that might affect trial participation (uncontrolled pulmonary, renal, or hepatic dysfunction, uncontrolled infection); Significant history of uncontrolled cardiac disease; i.e., uncontrolled hypertension, unstable angina, recent myocardial infarction (within prior 6 months), uncontrolled congestive heart failure, and cardiomyopathy that is either symptomatic or asymptomatic but with decreased ejection fraction <45%; Other significant co-morbid condition which the investigator feels might compromise effective and safe participation in the trial; Subject enrolled in another investigational device or drug trial, or is receiving other investigational agents; Concurrent or prior (within 7 days of trial day 1) anticoagulation therapy (low dose for port maintenance allowed); Concurrent radiation therapy is not permitted throughout the course of the trial; Inability to comply with the requirements of the trial; Pregnant or lactating women are excluded; Leptomeningeal disease or brain metastases requiring steroids or other therapeutic intervention.

An adverse event (AE) is an undesirable medical occurrence (e.g., sign, symptom, or diagnosis) or worsening of a pre-existing medical condition that occurs after the initial dose of 4-iodo-3-nitrobenzamide or irinotecan and as much as 30 days after the last dose of 4-iodo-3-nitrobenzamide or irinotecan or until another tumor treatment is initiated (whichever is first) whether or not it is considered to be related to the investigational product. A worsening of an existing medical condition occurs when a condition present at the time the informed consent form is signed (e.g., cancer, diabetes, migraine headaches, gout) becomes more severe, more frequent, or increased in duration during investigational product treatment. Hospitalizations for pre-treatment conditions (e.g., elective cosmetic procedures) or surgeries that were planned before entry into the study are not considered adverse events. Abnormal laboratory values should not be reported as an AE; however, any clinical consequences of the abnormality should be reported as an AE.

A serious adverse event (SAE) is defined by regulatory agencies as one that suggests a significant hazard or adverse event, regardless of the investigator's or sponsor's opinion on the relationship to investigational product. This includes, but may not be limited to, any event at any dose that: is fatal; is life threatening (places the subject at immediate risk of death); requires in-patient hospitalization or prolongation of existing hospitalization; results in persistent or significant disability/incapacity; is a congenital anomaly/birth defect; is any other significant medical hazard. A hospitalization is considered to meet the regulatory requirement for “serious” when it entails any inpatient hospital admission that requires an overnight stay. Elective hospitalizations for the administration of chemotherapy, or therapeutic or prophylactic transfusions are not considered SAEs. However, prolonged hospitalization or readmission, after the subject had already been discharged post-treatment, is considered an SAE. Any event that does not exactly meet this definition, but in the investigator's opinion represents a significant hazard (e.g., emergency room visit or outpatient surgery), can be assigned the “other significant hazard” regulatory reporting serious criteria. Additionally, important medical events that may not be immediately life threatening or result in death or hospitalization but that may jeopardize the subject or require intervention to prevent one of the outcomes listed above, or result in urgent investigation, may be considered serious. Examples include allergic bronchospasm, convulsions, and blood dyscrasias.

Efficacy Analyses: The overall response rate (ORR) is estimated by the proportion of patients who achieve an overall response (CR+PR).

Biomarker Analyses: Analyses to assess associations between differences in any pharmacogenomic results (e.g., BRCA) from samples taken before, during, and after 4-iodo-3-nitrobenzamide treatment are considered and offered by the subject on a voluntary basis.

Clinically applicable serum tumor markers are analyzed. Levels for carcinoembryonic antigen (CEA), CA-125, CA-19-9, or other as appropriate are measured.

Detailed PK analysis is done on samples from the phase 1 portion of the study. Blood level data obtained from the phase 1b portion of the study are listed.

Correlative analyses of markers of hypoxia (e.g. HIF-1, CA9, PHD3, PGKE and PNIP3) with clinical outcome are exploratory and descriptive in nature. Voluntary tumor biopsy samples at pre- and post-4-iodo-3-nitrobenzamide infusion are analyzed for the expression of hypoxia markers by using immunohistochemistry. Formalin-fixed paraffin tumor block from the prior surgery or biopsy is collected, and analyzed for the expression of hypoxia markers. The extent of DNA damage induced by irinotecan and presence of homologous DNA repair is analyzed by immunohistochemical staining of voluntarily obtained tumor biopsy samples at pre- and post-4-iodo-3-nitrobenzamide infusion. Immunohistochemical staining of tumor samples analyzes the formation of γ-H2AX foci and Rad51 foci. γ-H2AX foci are formed at the site of DNA double strand breaks, and the number of γ-H2AX per cell reflects the extent of DNA damage. Rad51 foci are formed at the DNA double strand break sites with presence of intact homologous DNA repair pathway. Defects in homologous DNA repair such as BRCA-deficiency result in absence of Rad51 foci formation in the presence of γ-H2AX foci.

### Modified RECIST Criteria

Definitions: Measurable disease—the presence of at least one measurable lesion. If the measurable disease is restricted to a solitary lesion, its neoplastic nature should be confirmed by cytology/histology. Measurable lesions—lesions that can be accurately measured in at least one dimension with longest diameter ≧20 mm using conventional techniques or ≧10 mm with spiral CT scan. Non-measurable lesions—all other lesions, including small lesions (longest diameter <20 mm with conventional techniques or <10 mm with spiral CT scan), i.e., bone lesions, leptomeningeal disease, ascites, pleural/pericardial effusion, inflammatory breast disease, lymphangitis cutis/pulmonis, cystic lesions, and also abdominal masses that are not confirmed and followed by imaging techniques.

All measurements should be taken and recorded in metric notation, using a ruler or calipers. All baseline evaluations should be performed as closely as possible to the beginning of treatment and never more than 14 days before study day 1. The same method of assessment and the same technique should be used to characterize each identified and reported lesion throughout the trial. Clinical lesions are only considered measurable when they are superficial (e.g., skin nodules and palpable lymph nodes). For the case of skin lesions, documentation by color photography, including a ruler to estimate the size of the lesion, is recommended.

Methods of Measurement: CT and MRI are the best currently available and reproducible methods to measure target lesions selected for response assessment.

Evaluation of Target Lesions—Complete Response (CR): Disappearance of all target lesions; Partial Response (PR): At least a 30% decrease in the sum of the LD of target lesions, taking as reference the baseline sum LD; Progressive Disease (PD): At least a 20% increase in the sum of the LD of target lesions, taking as reference the smallest sum LD recorded since the treatment started or the appearance of one or more new lesions; Stable Disease (SD): Neither sufficient shrinkage to qualify for PR nor sufficient increase to qualify for PD, taking as reference the smallest sum LD since the treatment started.

Evaluation of Non-Target Lesions—Complete Response (CR): Disappearance of all non-target lesions and normalization of tumor marker level; Incomplete Response/Stable Disease (SD): Persistence of one or more non-target lesion(s) or/and maintenance of tumor marker level above the normal limits; Progressive Disease (PD): Appearance of one or more new lesions and/or unequivocal progression of existing non-target lesions.

Evaluation of Best Overall Response: The best overall response is the best response recorded from the start of the treatment until disease progression/recurrence (taking as reference for PD the smallest measurements recorded since the treatment started). In general, the subject's best response assignment depends on the achievement of both measurement and confirmation criteria.

TABLE 3

| | | New | |
|---|---|---|---|
| Target Lesions | Non-Target Lesions | Lesions | Overall Response |
| CR | CR | No | CR |
| CR | Incomplete response/SD | No | PR |
| PR | Non-PD | No | PR |
| SD | Non-PD | No | SD |
| PD | Any | Yes or No | PD |
| Any | PD | Yes or No | PD |
| Any | Any | Yes | PD |

Subjects with a global deterioration of health status requiring discontinuation of treatment without objective evidence of disease progression at that time should be classified as having “symptomatic deterioration”.

Duration of Overall Response: The duration of overall response is measured from the time measurement criteria are met for CR or PR (whichever status is recorded first) until the first date that recurrence or PD is objectively documented, taking as reference for PD the smallest measurements recorded since the treatment started.

Duration of Stable Disease: SD is measured from the start of the treatment until the criteria for disease progression are met, taking as reference the smallest measurements recorded since the treatment started.

### Example 2

### Safety Assessment of Administration of 4-iodo-3-nitrobenzamide (BA) at Various Dosages

24 subjects (advanced solid tumors) were treated with 4-iodo-3-nitrobenzamide monotherapy at doses of 0.5, 1.0, 1.4, 2.8, 4.0, 5.6, and 8.0 mg/kg. Safety data indicated that 4-iodo-3-nitrobenzamide was well tolerated at all dose levels tested to date; no dose limiting toxicities (DLTs) were observed at any dose level. A total of 13 serious adverse events were reported for 5 trial participants. Any serious adverse events (SAEs) reported in the study were deemed not related to study drug by the study investigators. Best response measured to date was stable disease present through at least cycle 2 of the study for six subjects, with 1 subject not having reached end of cycle 2 for a disease assessment to be performed. One subject had completed 9 cycles of treatment with a staging of continued stable disease.

42 subjects (advanced solid tumors) were treated with 4-iodo-3-nitrobenzamide in combination with cytotoxic chemotherapeutics agents (topotecan, temozolomide, gemcitabine, or carboplatin/taxol) at 4-iodo-3-nitrobenzamide doses of 1.1, 2.0, 2.8, 4.0 and 5.6 mg/kg. Preliminary safety data indicated that 4-iodo-3-nitrobenzamide was well tolerated at all dose levels tested. Any serious adverse events reported in the study were deemed not related to study drug by the study investigators. Best response measured was stable disease present through at least cycle 2 of the study for eight subjects, stable disease through cycle 4 for three subjects, three subjects with partial response through at least cycle 2 and still on study, and one subject demonstrating a complete response thru cycle 6 (reached end of treatment). Several subjects enrolled did not yet reach end of cycle 2 for a disease assessment to be performed.

There were no SAEs attributed to 4-iodo-3-nitrobenzamide in either study. Safety data indicated that 4-iodo-3-nitrobenzamide would not cause any additional toxicities when combined with standard cytotoxic chemotherapy. Additionally, there was no evidence that 4-iodo-3-nitrobenzamide would potentiate any known toxicities associated with standard chemotherapeutic agents.

### Example 3

### A Phase 1b Study to Assess the Tolerability of the 4-iodo-3-nitrobenzamide (BA) in Combination with Irinotecan for the Treatment of Patients with Metastatic Breast Cancer (MBC)

This study was conducted to determine the maximum-tolerated dose (MTD) of irinotecan that can be used in combination with fixed dose 4-iodo-3-nitrobenzamide in patients with locally advanced or MBC.

Patients were treated with a 3+3 study design to determine safety of 4-iodo-3-nitrobenzamide (8 mg/kg IV twice-weekly on Days 1, 4, 8, and 11 every 21 days) in combination with escalating doses of irinotecan (80-125 mg/m² IV on Days 1 and 8 every 21 days).

Response rates were measured per modified RECIST criteria. Serial pharmacokinetic (PK) assessments were performed during Cycle 1, and archived paraffin-embedded tumor samples were collected to correlate markers of hypoxia (CAIX) with response.

The median age of 34 patients who received therapy was 50 years (range, 32-84), and the median number of prior therapies was 2 (range, 0-6). Preliminary analysis showed that tumors from 22 (65%) patients were ER−/PR−/HER2− (triple-negative), 10 (29%) were ER+/HER2−, and 1 (3%) was HER2+. One DLT of Grade 3 diarrhea with lower GI bleed was observed, thus establishing the optimal regimen from this study at 8 mg/kg 4-iodo-3-nitrobenzamide and 125 mg/m² irinotecan. Other toxicities included neutropenia, anemia and diarrhea. Of 26 patients evaluable for response, 5 (19%) had partial response (PR), 10 (38%) had stable disease (SD) for >4 cycles of therapy, and 9 (35%) developed progressive disease (PD) as best response. Of 10 patients who had reached 6 cycles at time of analysis, 5 (50%) had SD. Median follow-up time was 10 weeks.

Thus, an MTD of 8 mg/kg 4-iodo-3-nitrobenzamide and 125 mg/m² irinotecan was identified in patients with MBC. 4-iodo-3-nitrobenzamide in combination with irinotecan was well tolerated and showed efficacy, with evidence of clinical benefit (RR+SD≧6 cycles) in 50% in patients with treatment-refractory MBC.

### Example 4

### A Phase 1b Study to Assess the Safety and Tolerability of 4-iodo-3-nitrobenzamide (iniparib) in Combination with Irinotecan for the Treatment of Patients with Metastatic Breast Cancer (MBC)

The primary endpoints for this study were (1) safety and tolerability and (2) objective response rate (ORR). The secondary endpoint for this study was clinical benefit rate (CBR defined as the sum of ORR and stable disease (SD)>6 cycles).

Methods:

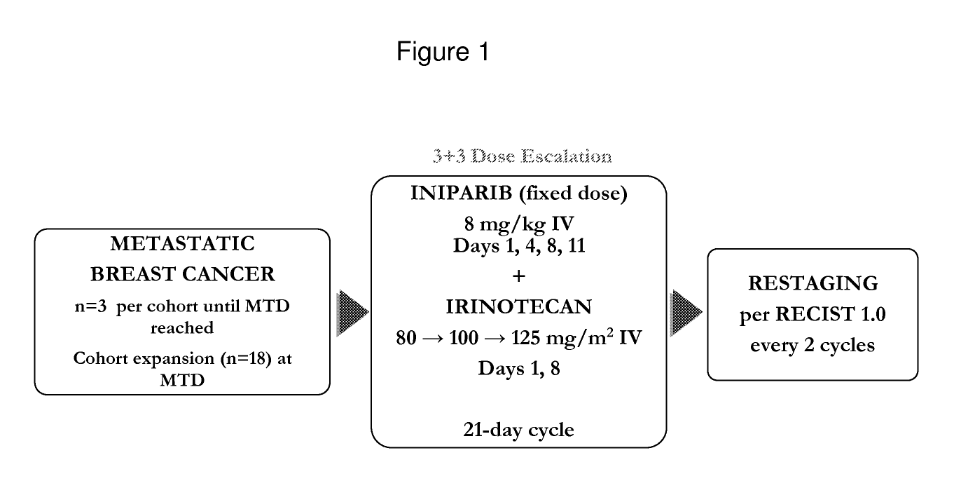

Patients were treated with a 3+3 study design to determine safety of 4-iodo-3-nitrobenzamide (8 mg/kg IV twice-weekly on Days 1, 4, 8, and 11 every 21 days) in combination with escalating doses of irinotecan (80-125 mg/m² IV on Days 1 and 8 every 21 days). The treatment scheme is shown in FIG. 1. ORR was measured per modified RECIST criteria. Serial pharmacokinetic (PK) assessments were performed during Cycle 1, and archived paraffin-embedded tumor samples were collected to correlate markers of hypoxia (CAIX) with response.

The key eligibility criteria for this study were (1) ≧18 years of age; (2) Histologically documented adenocarcinoma of the breast with progressing locoregional or metastatic disease and at least one bi-dimensionally measurable indicator lesion of at least 2.0 cm, as assessed by CT, MRI, or ultrasonography; (3) Prior treatment with at least one regimen containing an anthracycline, an anthraquinone, or a taxane; (4) Maximum of one adjuvant regimen and two regimens for metastatic disease, whether or not all of these were based on an anthracycline or taxane; (5) ECOG PS 0-2; (6) No symptomatic or untreated brain metastases requiring concurrent treatment; (7) Written, informed consent.

Patient characteristics are shown in Table 4.

TABLE 4
Patient Characteristics

| | 4-iodo-3-nitrobenzamide 8 mg/ | | |
|---|---|---|---|
| | kg + Irinotecan dose | | |
| | 80 mg/m² | 100 mg/m² | 125 mg/m² |
| N | 3 | 6 | 25 |
| Age, median (range) | 58 (48-78) | 52 (41-68) | 50 (32-84) |
| Race, n (%) | | | |
| White | 3 (100) | 5 (83.3) | 20 (80.0) |
| Black/African-American | 0 | 1 (16.7) | 5 (20.0) |
| ECOG PS, n (%) | | | |
| 0 | 2 (66.7) | 3 (50.0) | 11 (44.0) |
| 1 | 1 (33.3) | 3 (50.0) | 13 (52.0) |
| Missing/Unknown | 0 | 0 | 1 (4.0) |
| Receptor status, n (%) | | | |
| TN: ER−/PR−/HER2− | 3 (100) | 2 (33.3) | 17 (68.0) |
| HR+/HER2− | 0 | 3 (50.0) | 7 (28.0) |
| HR−/HER2+ | 0 | 0 | 1 (4.0) |
| Missing/Unknown | 0 | 1 (16.7) | 0 |
| Prior therapy, n (%) | | | |
| Chemotherapy | 3 (100) | 6 (100) | 25 (100) |
| Radiation therapy | 3 (100) | 6 (100) | 19 (76.0) |
| Hormonal | 0 | 4 (66.7) | 6 (24.0) |
| Surgery | 3 (100) | 6 (100) | 20 (80.0) |
| Other | 1 (33.3) | 0 | 8 (32.0) |

ECOG PS, Eastern Cooperative Oncology Group Performance Status;
TN, triple negative;
ER, estrogen receptor;
PR, progesterone receptor;
HER2, human growth hormone receptor-2;
HR, hormone receptor

Summary of Results:

The median age of 34 patients who received therapy was 50 years (range, 32-84), and the median number of prior therapies was 2 (range, 0-6). Tumors from 22 (64.7%) patients were ER−/PR−/HER2− (triple-negative), 10 (29.4%) were ER+/HER2−, and 1 (2.9%) was HER2+. One DLT of Grade 3 diarrhea with lower GI bleed was observed, thus establishing the optimal regimen from this study at 8 mg/kg 4-iodo-3-nitrobenzamide and 125 mg/m² irinotecan. Other toxicities included neutropenia, anemia and diarrhea. Of 22 evaluable patients at the highest irinotecan dose, 7 (31.8%) had an objective response, 6 (22.7%) had stable disease (SD)>4 cycles, 3 (13.6%) had SD>6 cycles, and 5 (22.7%) had progressive disease (PD) as best response. The clinical benefit rate (CBR, defined as ORR+SD>6 cycles) was 45.5%.

The efficacy data is shown in Table 5.

TABLE 5
Efficacy Summary

| | 4-iodo-3-nitrobenzamide 8 mg/kg + | | |
|---|---|---|---|
| | Irinotecan dose | | |
| | 80 mg/m² | 100 mg/m² | 125 mg/m² |
| | (N = 3) | (N = 6) | (N = 22) |
| | Number of patients (%) | | |
| Objective response rate (ORR) | 0 | 1 (16.7) | 7 (31.8) |
| Complete response | 0 | 0 | 1 (4.5) |
| Partial response | 0 | 1 (16.7) | 6 (27.3) |
| Stable disease | 1 (33.3) | 2 (33.3) | 10 (45.5) |
| Stable Disease >4 cycles | 1 (33.3) | 0 | 6 (22.7) |
| Stable Disease >6 cycles | 1 (33.3) | 0 | 3 (13.6) |
| Progressive disease | 2 (66.7) | 3 (50.0) | 5 (22.7) |
| Missing | 0 | 0 | 3 (13.6) |
| Clinical Benefit Rate | 1 (33.3) | 1 (16.7) | 10 (45.5) |
| (ORR + SD>6 cycles) | | | |

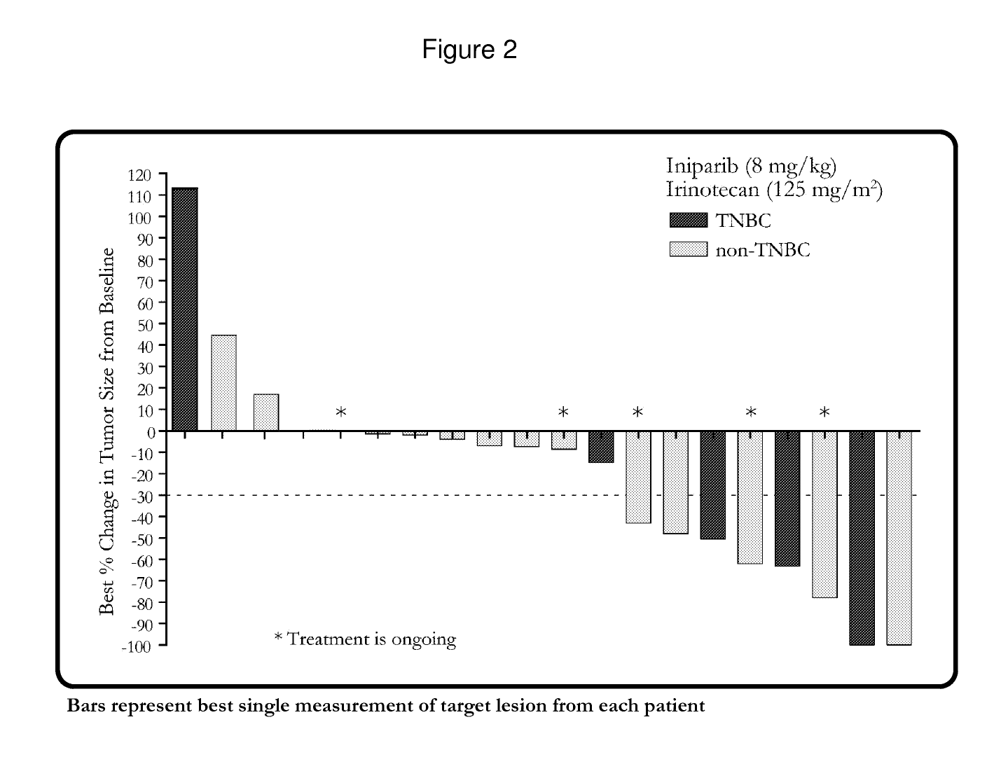

The effect of the treatment with 4-iodo-3-nitrobenzamide in combination with irinotecan on tumor size in triple negative breast cancer (“TNBC”) and non-TNBC is shown in FIG. 2.

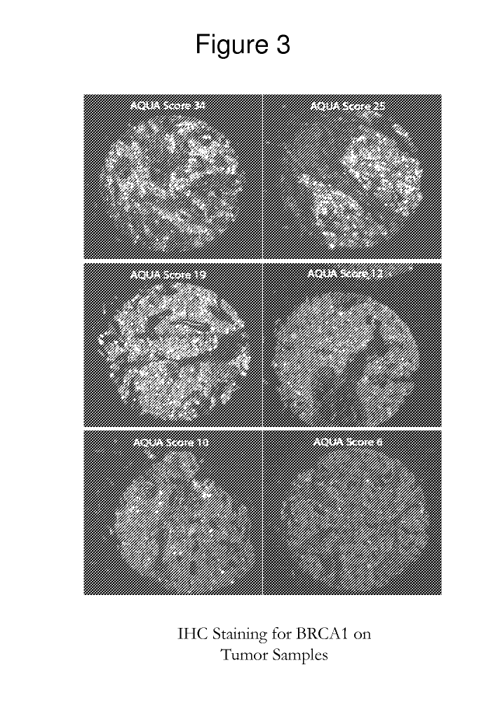

Tumoral BRCA1, ER, PR and HER2 were evaluated in 11 out of 30 patients. BRCA1 protein levels were measured using M110 (Ab-1; 1:1000; Calbiochem) with AQUA™ technology. Clinical benefit from 4-iodo-3-nitrobenzamide and irinotecan was observed in breast cancers that were ER+/PR+ and with varying levels of tumoral BRCA1 staining by AQUA™. See FIG. 3 and Table 6.

TABLE 6
BRCA1 Staining

| | Best | Max | Breast | | |
|---|---|---|---|---|---|
| | overall | tumor | cancer | Avg BRCA1 | Max BRCA1 |
| Patient | response | change | type | staining | staining |
| # 1 | CR | 100% decr | TN | 10.604 | 23.929 |
| # 2 | PR | 75% decr | ER+/PR+ | 15.158 | 23.79 |
| # 3 | PR | 50% decr | TN | 31.599 | 65.81 |
| # 4 | PR | 40% decr | ER+/PR+ | 17.732 | 39.804 |
| # 5 | SD | 5% decr | ER+/PR+ | 10.548 | 17.988 |
| # 6 | SD | 5% decr | TN | 12.228 | 51.855 |
| # 7 | SD | slight decr | TN | 7.194 | 17.799 |
| # 8 | SD | 5% incr | TN | 21.313 | 63.854 |
| # 9 | PD | 20% incr | TN | 13.36 | 42.625 |
| # 10 | PD | 60% incr | TN | 10.993 | 21.182 |

CR, complete response;
PR, partial response;
SD, stable disease;
PD, progressive disease;
TN, triple-negative;
decr: decrease;
incr: increase.

The safety data are summarized in Table 7.

TABLE 7
Treatment-emergent adverse events (TEAE) of interest

| 4-iodo-3-nitrobenzamide 8 mg/kg + Irinotecan Dose | | | | | | | |
|---|---|---|---|---|---|---|---|
| 80 mg/m2 | | 100 mg/m2 | | 125 mg/m2 | | All Patients | |
| (n = 3) | | (n = 6) | | (n = 25) | | (N = 34) | |
| Gr 3/4 | All grades | Gr 3/4 | All grades | Gr 3/4 | All grades | Gr 3/4 | All grades |

Number of patients (%)

HEMATOLOGIC

| | | | | | | | | |
|---|---|---|---|---|---|---|---|---|
| Neutropenia | 0 | 1 (33.3) | 1 (16.7) | 1 (16.7) | 8 (32.0) | 11 (44.0) | 9 (26.5) | 13 (38.2) |
| Leukopenia | 0 | 0 | 1 (16.7) | 1 (16.7) | 5 (20.0) | 9 (36.0) | 6 (17.6) | 10 (29.4) |
| Anemia | 0 | 1 (33.3) | 0 | 3 (50) | 2 (8.0) | 12 (48.0) | 2 (5.9) | 16 (47.1) |
| Hematocrit | 0 | 1 (33.3) | 0 | 2 (33.3) | 0 | 2 (8%) | 0 | 5 (14.7) |
| decreased | | | | | | | | |

NON-HEMATOLOGIC

| | | | | | | | | |
|---|---|---|---|---|---|---|---|---|
| Diarrhea | 0 | 1 (33.3) | 0 | 3 (50.0) | 3 (12.0) | 17 (68.0) | 3 (8.8) | 21 (61.8) |
| Nausea | 0 | 1 (33.3) | 0 | 4 (66.7) | 1 (4.0) | 16 (64.0) | 1 (2.9) | 21 (61.8) |
| Fatigue | 0 | 2 (66.7) | 0 | 4 (66.7) | 3 (12.0) | 14 (56.0) | 3 (8.8) | 20 (58.8) |
| Constipation | 0 | 2 (66.7) | 0 | 1 (16.7) | 1 (4.0) | 9 (36.0) | 1 (2.9) | 12 (35.3) |
| Any TEAE | 1 (33.3) | 2 (66.7) | 2 (33.3) | 4 (66.7) | 14 (56.0) | 20 (80.0) | 17 (50.0) | 26 (76.5) |

Conclusion:

The maximum protocol-defined dose combination was attained and tested in patients with metastatic breast cancer (“mBC”). No true MTD was reached. 4-iodo-3-nitrobenzamide in combination with irinotecan was well tolerated and was associated with a manageable rate of Grade 3/4 adverse events. Rates of Grade 3/4 neutropenia and diarrhea were lower than expected compared with reported data from single-agent irinotecan (Perez E A et al., J Clin Oncol. 2004, 22(14):2849-55). The highest dose evaluated (8 mg/kg 4-iodo-3-nitrobenzamide and 125 mg/m² irinotecan) was well tolerated and showed promising efficacy, with evidence of clinical benefit in 45.5% of patients with treatment-refractory MBC. Preliminary results from this study supported the promising safety and efficacy profile of 4-iodo-3-nitrobenzamide in combination with DNA-damaging chemotherapy.

### Example 5

### Phase 1/1b Dose Escalation Study Evaluating 4-iodo-3-nitrobenzamide (BA) as a Single Agent and in Combination With Irinotecan in Subjects With Advanced Solid Tumors

The purpose of this study is to assess the safety, establish the maximum tolerated dose (MTD) and generate pharmacokinetic profiles of 4-iodo-3-nitrobenzamide after IV administration in adult subjects with histologically documented advanced solid tumors that are refractory to standard therapy or for which no standard therapy is available. Additionally, the safety and tolerability and clinical response of 4-iodo-3-nitrobenzamide and irinotecan are investigated in patients with metastatic breast cancer in the phase 1b portion of the study. The primary outcome measure is maximum tolerated dose and the secondary outcome measure is clinical Response.

There are two arms for this study. Arm 1 includes 4-iodo-3-nitrobenzamide, i.v., twice weekly. Arm 2 includes 4-iodo-3-nitrobenzamide, i.v., twice weekly and irinotecan, i.v., weekly.

Inclusion criteria include the following: Pathologically documented, advanced solid tumor that is refractory to standard therapy or for which no standard therapy is available; ECOG performance status of 0, 1, or 2; Adequate hematological status; Any prior toxicity from prior chemotherapeutic treatment recovered to grade 1 or grade 0; 18 years of age or older; For phase 1b portion only: metastatic breast cancer.

Exclusion criteria include the following: Hematologic malignancies; Symptomatic or untreated brain metastases requiring concurrent treatment, inclusive of but not limited to surgery, radiation, and corticosteroids; Myocardial infarction within 6 months of study day 1, unstable angina, congestive heart failure with NYHA >class II, uncontrolled hypertension; Known positive test for HIV or hepatitis C virus, or chronic active hepatitis; Major surgery within 1 month of study day 1; History of second neoplasm, except for curatively treated non-melanoma skin cancer, carcinoma in situ of the cervix and other primary cancer with no known active disease present and no curative treatment administered for the last 3 years; History of seizure disorder or currently on anti-seizure medication; Systemic chemotherapy or radiation therapy within 28 days of study day 1; Antibody therapy for treatment of underlying malignancy within 1 month of study day 1; Evidence of liver disease shown by elevated enzymes; Evidence of renal disease shown by serum creatinine >1.5× upper limit of normal; Currently receiving platelet of GCF support for any medical condition; Concurrent use of herbal medications taken with the intent to treat cancer; Enrolled in or not yet completed at least 30 days since ending other investigational device or drug study.

### Example 6

### Treating Triple Negative Breast Cancer Brain Metastasis with 4-iodo-3-nitrobenzamide in Combination with Irinotecan

The purpose of the study is to investigate the response rate for triple negative breast cancer patients with brain metastasis when 4-iodo-3-nitrobenzamide is used in combination with irinotecan.

Primary outcome measure is efficacy as measured by intra or extra cranial time to progression (TTP). Secondary outcome measure is response rate as measured by RECIST.

Patients are treated with 4-iodo-3-nitrobenzamide at 5.6 mg/kg intravenously on days 1, 4, 8, and 11 and irinotecan at 125 mg/m² intravenously on days 1 and 8 of a 21-day treatment cycle.

The ages eligible for study are 21 years and older. The genders eligible for study are both male and female.

Inclusion criteria are: (1) Histologically-confirmed, ER negative, PR negative and Her2 non-overexpressing adenocarcinoma of the breast with brain lesion on radiographic imaging; (2) ECOG Performance Status of 0-2; (3) Life expectancy of >12 weeks; (4) No limit to prior therapies with last anti-cancer treatment ≧2 weeks from initiation of protocol-based therapy provided all toxicities (other than alopecia) have resolved to ≦Grade 1 or baseline; (5) No active serious infection or other comorbid illness which would impair ability to participate in the trial; (6) Stable or decreasing dose of steroids for ≧7 days; (7) Interval ≧4 weeks between open brain biopsy and initiation of protocol-based therapy; and (8) Patients must have adequate organ function.

Exclusion criteria are: (1) Pregnant or breast-feeding; (2) Prior allergic reaction to 4-iodo-3-nitrobenzamide; (3) Prior allergic reaction to irinotecan; (4) Evidence of hemorrhage or impending herniation on baseline brain imaging; (5) Evidence of diffuse leptomeningeal disease on brain MRI or by previously documented CSF cytology-NOTE: discrete dural metastases are permitted; (6) Clinically significant cardiac, renal, hepatic, infectious or pulmonary disease which might affect trial participation; (7) Concurrent or planned radiation, hormonal, chemotherapeutic, experimental or targeted biologic therapy; (8) Contraindication to gadolinium-enhanced MRI imaging; and (9) Inability to comply with study and/or follow-up procedures.

### Example 7

### Phase II Study of 4-iodo-3-nitrobenzamide Plus Irinotecan to Treat Triple Negative Breast Cancer (“TNBC”) Brain Metastases (“BM”)

This study is conducted to evaluate the effect of 4-iodo-3-nitrobenzamide in combination with irinotecan in patients with TNBC brain metastases. Patients with TNBC BM measuring >0.5 cm are eligible. There are no limits to prior therapies, including 4-iodo-3-nitrobenzamide; stable or decreasing steroids ≧7 days prior to study entry. Patients with leptomeningeal disease are excluded. Patients receive irinotecan (125 mg/m² IV Days 1, 8) prior to 4-iodo-3-nitrobenzamide (5.6 mg/kg IV Days 1, 4, 8, and 11) of a 21-day cycle.

Detailed inclusion criteria for this study are: 1) Histologically-confirmed, ER negative, PR negative and Her2 negative (0-1+ or FISH non-amplified; by clinical assay on either primary or metastatic tumor) adenocarcinoma of the breast with brain lesion on radiographic imaging; 2) Cohort 1: Patients with unequivocal evidence of new and/or progressive brain metastases (>5.0 mm in longest dimension) on radiographic imaging after prior intracranial radiation therapy (i.e. WBRT, SRS, GK or local equivalent), or Cohort 2: Intracranial radiation-naïve patients for whom intracranial radiation therapy (“ICR”) is not emergently indicated (at treating physician's discretion) with a >5.0 mm (in longest dimension) brain lesion on radiographic imaging within 2 weeks of initiation of protocol-based therapy; 3) ECOG Performance Status of 0-2; 4) Life expectancy of >12 weeks; 5) No limit to prior therapies with last anti-cancer treatment ≧2 weeks from initiation of protocol-based therapy provided all toxicities (other than alopecia) have resolved to ≦Grade 1 or baseline; 6) No active serious infection or other comorbid illness which would impair ability to participate in the study; 7) Stable or decreasing dose of steroids for ≧7 days; 8) Interval ≧4 weeks between open brain biopsy and initiation of protocol-based therapy; 9) Patients must have adequate organ function as evidenced by: Absolute neutrophil count ≧1.5 μL; Platelet count ≧100,000 μL; Bilirubin ≦1.5 X's upper limit of normal (ULN); AST or ALT≦2.5 X's ULN (≦5 X's ULN if liver metastases are present); Creatinine clearance ≧30 mL/min; 10) Not pregnant or nursing and able to use appropriate contraception; 11) At least 21 years of age; 12) Archived, paraffin-embedded tissue block (primary or metastatic) available for genomic studies required; 13) Signed, Institutional Review Board (“IRB”) approved written informed consent.

Detailed exclusion criteria for this study are: 1) Pregnant or breast-feeding; 2) Prior allergic reaction to 4-iodo-3-nitrobenzamide; 3) Prior allergic reaction to irinotecan; 4) Evidence of hemorrhage or impending herniation on baseline brain imaging; 5) Evidence of diffuse leptomeningeal disease on brain MRI or by previously documented CSF cytology (discrete dural metastases are permitted); 6) Clinically significant cardiac, renal, hepatic, infectious or pulmonary disease which might affect trial participation; 7) Concurrent or planned radiation, hormonal, chemotherapeutic, experimental or targeted biologic therapy; 8) Contraindication to gadolinium-enhanced MRI imaging; 9) Inability to comply with study and/or follow-up procedures; 10) Patients unable or unwilling to discontinue (and substitute if necessary) use of CYP3A4 inducing drugs, including the anti-convulsants (e.g. phenyloin, phenobarbital or carbamazepine) and strong CYP3A4 inhibiting drugs (e.g., ketoconazole) as these can significantly change plasma concentrations of irinotecan and its active metabolites (patients must not have received any of the prohibited drugs for at least 2 weeks prior to Day 1 of study drug administration).

Intra- and extracranial disease is assessed every 9 weeks (wks) by gadolinium-enhanced brain MRI and CT chest/abdomen/pelvis, respectively. The co-primary endpoint is intracranial (modified RECIST) and extracranial (RECIST 1.1) time to progression (“TTP”). Secondary objectives include CNS and non-CNS response rates, progression free survival, overall survival, quality of life, and correlative science endpoints. Time to progression is defined as the time from treatment initiation to documented disease progression as defined below. Progression free survival (PFS) is defined as the time from the start of treatment until documented disease progression as defined below. Overall survival (OS) is defined as the time from the start of treatment until death due to any cause. Intracranial response is also assessed by 3-dimensional (3-D) estimates of tumor volume, and by CNS composite response criteria.

### Assessment of Intracranial Disease

Tumor Measurement: Intracranial tumor lesions are evaluated via gadolinium-enhanced brain MRI. Measurable disease is defined as the presence of at least one measurable brain lesion that can be accurately measured in at least one dimension (longest diameter to be recorded) as >5.0 mm via gadolinium-enhanced brain MRI. The same method of assessment and the same techniques are used to characterize each identified and reported lesion at baseline and during follow-up. A maximum of 5 target lesions are identified and followed during the course of study.

Modified RECIST Criteria for Evaluation of Intracranial Disease: Complete Response (CR)—Disappearance of all target lesions; Partial Response (PR)—at least a 30% decrease in the sum of the longest diameter (LD) of target lesions, taking as reference the baseline sum longest diameter and an absolute decrease of at least 5 mm in at least one target lesion; Stable Disease (SD)—neither sufficient shrinkage to qualify for partial response nor sufficient increase to qualify for progressive disease, taking as reference the smallest sum longest diameter since the treatment started; Progressive Disease (PD)—at least a 20% increase in the sum LD of target lesions, taking as reference the smallest sum longest diameter recorded since the treatment started and an absolute increase in size of at last 5 mm in at least one target lesion or the appearance of one or more new lesions of at least 6 mm in size.

CNS Composite Response Criteria: CNS Response—≧50% volumetric reduction of target CNS lesion(s) as measured via volumetric MRI in the absence of: new lesions, progression in any non-target CNS lesions, the need for increased dose of steroids, progressive neurological signs/symptoms, or progressive extracranial disease; CNS Disease Progression—Either a ≧40% volumetric increase from nadir in target CNS lesion(s) as measured via volumetric MRI, an increase in steroid requirements, or progression of neurological signs/symptoms. Assessment of Extracranial Disease-Measurement Based on RECIST 1.1

Extracranial tumor lesions are evaluated via CT scan of the chest, abdomen and pelvis (and bone scan if clinically indicated). Measurable disease is defined as the presence of at least one measurable lesion that can be accurately measured in at least one dimension with the longest diameter a minimum size of: ≧10 mm by CT scan (CT scan slice thickness no greater than 5 mm); 10 mm caliper measurement by clinical exam (lesions which cannot be accurately measured with calipers should be recorded as non-measurable); 20 mm by chest x-ray. For malignant lymph nodes to be considered pathologically enlarged and measurable, a lymph node must be ≧15 mm in short axis when assessed by CT scan (CT scan slice thickness recommended to be no greater than 5 mm). At baseline and in follow-up, only the short axis is measured and followed. All other lesions, including small lesions (longest diameter <10 mm or pathological lymph nodes with ≧10 to <15 mm short axis) as well as truly non-measurable lesions, are considered non-measurable. Lesions considered truly non-measurable include: leptomeningeal disease; ascites; pleural/pericardial effusion; inflammatory breast disease; lymphangitic involvement of skin or lung, abdominal masses/abdominal organomegaly identified by physical exam that is not measurable by reproducible imaging techniques.

### Baseline Documentation of Extracranial Target and Non-Target Lesions

All measurable lesions up to a maximum of 5 lesions total (and a maximum of two lesions per organ) representative of all involved organs are identified as target lesions and recorded and measured at baseline. Target lesions are selected on the basis of their size (lesions with the longer diameter), are representative of all involved organs, and in addition are those that lend themselves to reproducible repeated measurements. A sum of the diameters (longest for non-nodal lesions, short axis for nodal lesions) for all target lesions is calculated and reported as the baseline sum diameters. If lymph nodes are to be included in the sum, only the short axis is added into the sum. The baseline sum diameters are used as reference to further characterize the objective tumor response of the measurable dimension of the disease. All other lesions (or sites of disease) including pathological lymph nodes are identified as non-target lesions and recorded at baseline.

### RECIST 1.1 Criteria for Evaluation of Extracranial Disease

Evaluation of Target Lesions: Complete response (CR)—Disappearance of all target lesions (any pathological lymph node (LN) target or no must have decreased in short axis to <10 mm); Partial response (PR)—At least a 30% decrease in the sum of the LD of the target lesions taking as reference the baseline sum LD; Progressive Disease (PD)—At least a 20% increase in the sum of the LD of the target lesions taking as reference the smallest sum LD recorded since the treatment started including baseline if that is the smallest on study (in addition to the relative increase of 20%, the sum must also demonstrate an absolute increase of at least 5 mm) (the appearance of one or more new lesions also constitutes PD); Stable disease (SD)—Neither sufficient shrinkage to qualify for PR nor sufficient increase to qualify for PD taking as references the smallest sum LD since the treatment started.

Evaluation of Non-Target Lesions: Complete response (CR)—Disappearance of all non-target lesions and normalization of tumor marker levels (all LN must be non-pathological in size (<10 mm short axis)); Non-complete response (non-CR)/non-progression (non-PD)—Persistence of one or more non-target lesion(s) or/and maintenance of tumor marker level above the normal limits; Progressive disease (PD)—Appearance of one or more new lesions and/or unequivocal progression of existing non-target lesions.

Evaluation of Best Overall Response: The best overall response is the best response recorded from the start of the study treatment until the end of treatment provided the confirmation criteria are met. To be assigned a status of PR or CR, changes in tumor measurements must be confirmed by repeat studies that should be performed >4 weeks after the criteria for response are first met. If a CR/PR cannot be confirmed, the original “response” should be considered stable disease. The best overall response is defined according to Table 8:

TABLE 8

| | Overall Response | |
|---|---|---|
| Overall Response | Subsequent | |
| First Time Point | Time Point | BEST Overall Response |
| CR | CR | CR |
| CR | PR | SD, PD, or PR¹ |
| CR | SD | SD provided minimum criteria for |
| | | SD duration met, otherwise, PD |
| CR | PD | SD provided minimum criteria for |
| | | SD duration met, otherwise, PD |
| CR | NE₂ | SD provided minimum criteria for |
| | | SD duration met, otherwise, NE₂ |
| PR | CR | PR |
| PR | PR | PR |
| PR | SD | SD |
| PR | PD | SD provided minimum criteria for |
| | | SD duration met, otherwise, PD |
| PR | NE₂ | SD provided minimum criteria for |
| | | SD duration met, otherwise, NE₂ |
| NE | NE₂ | NE₂ |

¹If a CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in fact the patient had PR, not CR at the first time point. Under these circumstances, the original CR should be changed to PR and the best response is PR.
²NE = inevaluable.

Statistical considerations: A sample size of 32 evaluable patients has 80% power to detect a difference between the null (2.0 months TTP) and the alternative hypothesis (3.15 months, 57.5% improvement) at a 0.05 significance level. Assuming a 20% drop-out rate, 40 patients are enrolled.

Correlative studies: Brain magnetic resonance angiography pre- and 9 wks post-therapy to determine if intracranial vasculature dynamics predict response. Archival primary and/or metastatic tissues are required from all patients to evaluate intrinsic breast cancer subtype and alterations in DNA repair genes. Among patients who consent for testing, BRCA1/2 status is correlated with response. Optional pre- and post-therapy (non-CNS) biopsies are expected from 25% of patients to assess in vivo effects on proliferation, apoptosis, and gene expression.

Although the foregoing invention has been described in some detail by way of illustration and example for purposes of clarity of understanding, the descriptions and examples should not be construed as limiting the scope of the invention.

### Abbreviations

“(%) TGD:” (percent) tumor growth delay; “AE:” Adverse event; “AUC:” Area under the plasma concentration-time curve; “BA:” 4-iodo-3-nitrobenzamide; “biwk to end:” twice weekly (dosing) for the duration of the study; “BUN:” Blood urea nitrogen; “BW:” body weight; “CBC:” complete blood count; “CHF:” Congestive heart failure; “CL:” Clearance; “C1r:” Renal clearance; “Cmax:” Maximum observed concentration; “CNS:” Central nervous system; “CO2:” Carbon dioxide; “CR:” complete regression or complete response; “CrCl24 hr:” 24 hour creatinine clearance; “CRF:” Case report form; “CT:” Computed tomography; “CTC:” Circulating tumor cells; “CTCAE:” Common terminology criteria for adverse events; “Cut:” Concentration in urine for time; “D:” Day (of the study); “dC/dtmax:” Maximum rate of concentration change; “DCE-MRI:” Dynamic contrast enhanced magnetic resonance imaging; “DLT:” Dose limiting toxicity; “DNA:” Deoxyribonucleic acid; “ECHO:” Echocardiogram; “ECOG:” Eastern Cooperative Oncology Group; “EKG:” Electrocardiogram; “End of Study (EOS):” Last dose of 4-iodo-3-nitrobenzamide+30 days; “Enrollment:” Study day 1 when 4-iodo-3-nitrobenzamide first administered; “FDA:” Food and Drug Administration; “FIH:” First in human; “GBM:” glioblastoma multiforme; “GCP:” Good clinical practice; “GLU:” Glucose; “HCT:” Hematocrit; “HED:” Human equivalent dose; “Hgb:” Hemoglobin; “HPBCD:” 25% hydroxypropyl-β-cyclodextrin; “i.p.:” intraperitoneal(ly); “IC50:” 50% inhibitory concentration; “IC90:” 90% inhibitory concentration; “ICH:” International Conference on Harmonization; “In vitro:” In an artificial environment; “In vivo:” Within the living body; “iniparib:” 4-iodo-3-nitrobenzamide (BA); “IRB:” Institutional review board; “IV:” Intravenous; “K₂EDTA:” potassium ethylenediaminetetraacetic acid; “LD:” Longest diameter; “MedRA:” Medical Dictionary for Regulatory Submissions; “Mins:” Minutes; “MRI:” Magnetic resonance imaging; “MTD:” maximum tolerated dose; “MTV (n):” median tumor volume in mm3 of the number of animals, n, remaining on the last day of the study; “MTV:” median tumor volume; “MUGA:” Multiple gated acquisition; “n:” number of mice in a group per protocol; number of evaluable mice in a group for analyses; “ne:” not evaluated; “NOAEL:” No observable adverse effect level; “ns:” not significant; “NTR:” non-treatment-related (death); “NTRm:” non-treatment-related (death) due to metastasis and/or tumor invasion; “NYHA:” New York Heart Association; “OMP:” miniature osmotic infusion pump; “p.o.:” by mouth (per os); “PARP:” poly (ADP-ribose) polymerase; “PD:” Pharmacodynamic or Progressive Disease; “PK:” Pharmacokinetic; “PLT:” Platelets; “PR:” partial regression or partial response; PT:” Prothrombin time; “PTT:” Partial thromboplastin time; “Q2W:” Twice weekly dosing; “qd×5:” once daily (dosing) for five days; “QD:” Once daily dosing; “QT:” Part of electrocardiographic wave representing ventricular repolarization; “QTc:” Corrected QT; “Rad:” Randomization; “RBC:” Red blood cell; “RECIST:” Response evaluation criteria in solid tumor; “RNA:” Ribonucleic acid; “s.c.:” subcutaneous(ly); “SAE:” Serious adverse event; “Screening:” Point where subject signs the informed consent form; “SD:” Stable disease; “SMC:” Safety Monitoring Committee; “STD:” Severe toxic dose; “STD10:” One-tenth of the severe toxic dose; “Study Day 1:” Day 4-iodo-3-nitrobenzamide is first administered; “T½:” Terminal elimination half-life; “TBILI:” Total bilirubin (direct and indirect); “TFS:” tumor-free survivor(s); “TGI:” tumor growth inhibition; “Tmax:” Time to reach maximum plasma concentration; “TP:” Total protein; “TR:” treatment-related (death); “TTE:” time to endpoint; “ULN:” Upper limit of normal; “US:” Ultrasound; “WBC:” White blood cell.

1. A method of treating a patient with breast cancer brain metastasis comprising administering to the patient an effective amount of (a) 4-iodo-3-nitrobenzamide or a metabolite thereof or a pharmaceutically acceptable salt thereof and (b) irinotecan or a pharmaceutically acceptable salt thereof, wherein the breast cancer is ER-negative, PR-negative, and HER2-nonoverexpressing.

2. The method of claim 1, wherein the effective amount is administered over a 21-day treatment cycle, wherein 4-iodo-3-nitrobenzamide or a metabolite thereof or a pharmaceutically acceptable salt thereof is administered to the patient at about 5.6 mg/kg on days 1, 4, 8, 11 of the treatment cycle, and wherein irinotecan or a pharmaceutically acceptable salt thereof is administered at about 125 mg/m² on days 1 and 8 of the cycle.

3. The method of claim 1, wherein the effective amount is administered over a 21-day treatment cycle, wherein 4-iodo-3-nitrobenzamide or a metabolite thereof or a pharmaceutically acceptable salt thereof is administered to the patient at about 11.2 mg/kg on days 1 and 8 of the treatment cycle, and wherein irinotecan or a pharmaceutically acceptable salt thereof is administered at about 125 mg/m² on days 1 and 8 of the cycle.

4. A method of treating locally advanced or metastatic breast cancer in a patient, comprising administering to the patient having locally advanced or metastatic breast cancer an effective amount of (a) 4-iodo-3-nitrobenzamide or a metabolite thereof or a pharmaceutically acceptable salt thereof, and (b) irinotecan or a pharmaceutically acceptable salt thereof, wherein the method comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle.

5. The method of claim 4, wherein the patient has a breast cancer that is negative for human epidermal growth factor 2 receptor (“HER2-neu”).

6. The method of claim 4, wherein the patient has a breast cancer that is positive for HER2-neu.

7. The method claim 4, wherein the patient has a breast cancer that is negative for estrogen receptor (“ER”).

8. The method of claim 4, wherein the patient has a breast cancer that is positive for ER.

9. The method of claim 4, wherein the patient has a breast cancer that is negative for progresterone receptor (“PR”).

10. The method of claim 4, wherein the patient has a breast cancer that is positive for PR.

11. The method of claim 4, wherein the breast cancer is locally advanced breast cancer.

12. The method of claim 4, wherein the breast cancer is metastatic breast cancer.

13. The method of claim 12, wherein the metastasis comprises brain metastases.

14. The method of claim 13, wherein the brain metastasis is at least about 0.5 centimeter.

15. The method of claim 14, wherein the brain metastasis is new brain metastasis after radiation therapy.

16. The method of claim 14, wherein the brain metastasis is progressive brain metastasis after radiation therapy.

17. The method of claim 4, wherein irinotecan is administered at about 125 mg/m².

18. The method of claim 17, wherein 4-iodo-3-nitrobenzamide is administered at about 8 mg/kg on days 1, 4, 8, and 11 of the 21-day cycle, and wherein irinotecan is administered at about 125 mg/m² on days 1 and 8 of the 21-day cycle.

19. The method of claim 1, wherein the patient has breast adenocarcinoma.

20. The method of claim 4, wherein the breast cancer is locoregional.

21. The method of claim 4, wherein the patient has distant metastasis.

22. The method of claim 1, wherein the patient has systemic metastasis.

23. The method of claim 1, wherein the patient has received prior chemotherapy treatment comprising at least one regimen selected from the group consisting of an anthracycline, an anthraquinone, and a taxane.

24. The method of claim 23, wherein the patient is refractory to at least one regimen selected from the group consisting of an anthracycline, an anthraquinone, and a taxane.

25. The method of claim 1, wherein 4-iodo-3-nitrobenzamide or the metabolite thereof or the pharmaceutically acceptable salt thereof is administered intravenously.

26. The method of claim 1, wherein irinotecan or the pharmaceutically acceptable salt thereof is administered intravenously.

27. The method of claim 1, wherein the method further comprises surgery, radiation therapy, chemotherapy, gene therapy, DNA therapy, viral therapy, RNA therapy, adjuvant therapy, neoadjuvant therapy, immunotherapy, nanotherapy or a combination thereof.

28. The method of claim 27, wherein the radiation therapy comprises administering to the patient gamma irradiation.

29. The method of claim 1, wherein the effective amount produces at least one therapeutic effect selected from the group consisting of reduction in size of a breast tumor, reduction in metastasis, complete remission, partial remission, stable disease, and a pathologic complete response.

30. A kit for treating a patient with breast cancer brain metastasis comprising (a) 4-iodo-3-nitrobenzamide or a metabolite thereof or a pharmaceutically acceptable salt thereof, and (b) irinotecan or a pharmaceutically acceptable salt thereof, wherein the breast cancer is ER-negative, PR-negative, and HER2-nonoverexpressing.

31. The kit of claim 30 further comprising a package insert or label containing instructions for using the effective amount of (a) 4-iodo-3-nitrobenzamide or a metabolite thereof or a pharmaceutically acceptable salt thereof, and (b) irinotecan or a pharmaceutically acceptable salt thereof is administered to the patient to treat the patient with breast cancer brain metastasis.

32. The kit of claim 31, wherein the effective amount is administered over a 21-day treatment cycle, wherein 4-iodo-3-nitrobenzamide or a metabolite thereof or a pharmaceutically acceptable salt thereof is administered to the patient at about 5.6 mg/kg on days 1, 4, 8, 11 of the treatment cycle, and wherein irinotecan or a pharmaceutically acceptable salt thereof is administered at about 125 mg/m² on days 1 and 8 of the cycle.

33. The kit of claim 31, wherein the effective amount is administered over a 21-day treatment cycle, wherein 4-iodo-3-nitrobenzamide or a metabolite thereof or a pharmaceutically acceptable salt thereof is administered to the patient at about 11.2 mg/kg on days 1 and 8 of the treatment cycle, and wherein irinotecan or a pharmaceutically acceptable salt thereof is administered at about 125 mg/m² on days 1 and 8 of the cycle.

34. A kit comprising (a) 4-iodo-3-nitrobenzamide or a metabolite thereof or a pharmaceutically acceptable salt thereof, and (b) a package insert or label containing instructions for using an effective amount of 4-iodo-3-nitrobenzamide or a metabolite thereof or a pharmaceutically acceptable salt thereof, in combination with irinotecan or a pharmaceutically acceptable salt thereof to treat a patient with breast cancer brain metastasis, wherein the breast cancer is ER-negative, PR-negative, and HER2-nonoverexpressing.

35. The kit of claim 34, wherein the effective amount is administered over a 21-day treatment cycle, wherein 4-iodo-3-nitrobenzamide or a metabolite thereof or a pharmaceutically acceptable salt thereof is administered to the patient at about 5.6 mg/kg on days 1, 4, 8, 11 of the treatment cycle, and wherein irinotecan or a pharmaceutically acceptable salt thereof is administered at about 125 mg/m² on days 1 and 8 of the cycle.

36. The kit of claim 34, wherein the effective amount is administered over a 21-day treatment cycle, wherein 4-iodo-3-nitrobenzamide or a metabolite thereof or a pharmaceutically acceptable salt thereof is administered to the patient at about 11.2 mg/kg on days 1 and 8 of the treatment cycle, and wherein irinotecan or a pharmaceutically acceptable salt thereof is administered at about 125 mg/m² on days 1 and 8 of the cycle.

37. A kit for treating locally advanced or metastatic breast cancer in a patient comprising (a) 4-iodo-3-nitrobenzamide or a metabolite thereof or a pharmaceutically acceptable salt thereof, (b) irinotecan or a pharmaceutically acceptable salt thereof, and (c) a package insert or label containing instructions for using 4-iodo-3-nitrobenzamide or a metabolite thereof or a pharmaceutically acceptable salt thereof, and irinotecan or a pharmaceutically acceptable salt thereof to treat locally advanced or metastatic breast cancer in the patient, wherein the treatment comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle.

38. A kit comprising (a) 4-iodo-3-nitrobenzamide or a metabolite thereof or a pharmaceutically acceptable salt thereof, and (b) a package insert or label containing instructions for using 4-iodo-3-nitrobenzamide or a metabolite thereof or a pharmaceutically acceptable salt thereof, in combination with irinotecan or a pharmaceutically acceptable salt thereof to treat locally advanced or metastatic breast cancer in a patient, wherein the treatment comprises at least one cycle, wherein the cycle is a period of 21 days, wherein 4-iodo-3-nitrobenzamide or the pharmaceutically acceptable salt thereof is administered at about 8 mg/kg twice weekly for two weeks of the cycle, and wherein irinotecan or a pharmaceutically acceptable salt thereof is administered at about 80 mg/m² to about 125 mg/m² once weekly for two weeks of the cycle.
